(19) United States
(12) Patent Application Publication
(10) Pub. No.: US 2021/0024639 A1
(43) Pub. Date: Jan. 28, 2021

(54) METHODS FOR PROMOTING PANCREATIC ISLET CELL GROWTH

(71) Applicant: AGOMAB THERAPEUTICS, GENT (BE)

(72) Inventor: Paolo MICHIELI, Rivalta di Torino (IT)

(21) Appl. No.: 16/960,261

(22) PCT Filed: Jan. 3, 2019
(86) PCT No.: PCT/EP2019/050084

§ 371 Date: Jul. 6, 2020

(30) Foreign Application Priority Data
Jan. 3, 2018 (IT) 102018000000534

(51) Int. Cl. C07K 16/28; A61K 35/39; A61K 38/28; A61P 5/50; A61K 39/395

(52) U.S. Cl. CPC C07K 16/2863; A61K 35/39; A61K 38/28; A61P 5/50; C07K 16/2809; A61K 39/3955; A61K 2039/507; C07K 2317/55; C07K 2317/565; C07K 2317/56; C07K 2317/567; C07K 2317/24; C07K 2317/92; C07K 2317/22

(57) ABSTRACT

The present invention relates to methods of promoting growth of pancreatic islet cells, especially beta islet cells. In particular, the invention relates to methods of promoting growth of pancreatic islet cells by administration of HGF-MET agonists, such as MET agonist antibodies or fragments thereof. The invention further relates to HGF-MET agonists, such as MET agonist antibodies or fragments thereof, and pharmaceutical compositions comprising said agonists, for use in methods of the invention.

### FIELD OF THE INVENTION

The present invention relates to methods of promoting growth of pancreatic islet cells, especially beta islet cells. In particular, the invention relates to methods of promoting growth of pancreatic islet cells by administration of HGF-MET agonists, such as MET agonist antibodies or fragments thereof. The invention further relates to HGF-MET agonists, such as MET agonist antibodies or fragments thereof, and pharmaceutical compositions comprising said agonists, for use in methods of the invention.

### BACKGROUND

The pancreatic islets, or islets of Langerhans, are regions of endocrine tissues and cells situated in the pancreas in so-called “density routes”. Pancreatic islets include alpha, beta, gamma, delta, and epsilon cells, each playing a role in the endocrine activity of the pancreas. In particular, alpha and beta cells are especially important in the regulation of blood glucose levels.

Type 1 diabetes is an autoimmune disease characterized by immune-mediated destruction of pancreatic cells in the islets of Langerhans, especially beta islet cells. This progressive degeneration leads to impairment of insulin production, thus causing high blood glucose levels. Typically, the onset of clinical symptoms is associated with 80-95% reduction in beta cell mass (Klinke, PloS One 3:e1374, 2008). Regenerating beta cells and protecting them from the progressive destruction by immune system is a key unmet medical need in diabetic patients and the Holy Grail in diabetes research.

Although characterized by different etiological mechanisms, type 2 diabetes also leads to Langerhans islet degeneration. In fact, type 2 diabetes is characterized by aberrant insulin production in the presence of insulin resistance, leading to high blood glucose levels and inability of beta cells to compensate for the increased demand of insulin (Christoffersen et al., Am J Physiol Regul Integr Comp Physiol 297:1195-201, 2009). In type 2 diabetes, beta islet cells exhibit defective insulin production and, in late stage disease, the cells themselves can degenerate.

Current management of patients suffering from degeneration of pancreatic islet cells, such as diabetes patients, uses dietary control, with or without administration of insulin. However, this approach does not affect the underlying pathophysiology of the conditions. Novel therapies are therefore needed.

### SUMMARY OF THE INVENTION

It is surprisingly identified herein that MET agonists promote growth of pancreatic islet cells. Moreover, the generated pancreatic islet cells were functional, leading to restoration of insulin production and normalization of glycaemia.

Growth and regeneration of pancreatic islet cells is particularly important in treating diabetes, where the underlying pathophysiology can be treated by the methods described herein. This is a significant improvement on current management of the condition, which simply attempts to control the symptoms.

Promoting growth of pancreatic islet cells is especially important when treating patients in the early stages of type 1 diabetes. Typically, type 1 diabetes symptoms become manifest at adolescence. However, when the pathology is diagnosed, the majority of the patient's pancreatic beta cells have been destroyed (greater than 50%, for example 70% or 80% destruction). Langerhans islet cell degeneration occurs rapidly—as a result, the time-window for effective therapeutic intervention is narrow.

For example, immuno-suppressive drugs are being investigated as therapy for newly-diagnosed type 1 diabetes patients, in an effort to reduce the autoimmune-mediated islet cell destruction. However, immunosuppressants require several months before showing the first clinical benefits. When this occurs, approximately half year after treatment start, the beta cells of the pancreas continue to be destroyed, often completely. As a result, the use of the immunosuppressants is in vain. Maintaining islet (beta) cells during this crucial window is a highly unmet medical need for diabetes patients.

Surprisingly, as demonstrated herein, MET agonists (for example MET agonist antibodies) not only maintain pancreatic islet cell populations, but are able to promote their growth and regeneration. Although animals transgenically overexpressing HGF have been described as exhibiting altered beta cell growth, it was unknown and unclear whether an exogenous, non-native MET-binding agonist would have any effect. It is surprisingly shown herein that administration of a non-native MET agonist can not only maintain islet cell levels in diabetes, but promote their growth and regeneration. Provision of a clinical therapeutic agent able to promote pancreatic islet cell growth has been a long-felt need in diabetes therapy that is solved for the first time herein.

Accordingly, in a first aspect is provided a method of promoting pancreatic islet cell growth comprising administering to a subject an HGF-MET agonist.

In a further aspect is provided a method of promoting insulin production in a subject exhibiting depressed insulin production, comprising administering to a subject an HGF-MET agonist. In a preferred embodiment, the method is characterised by inducing increased pancreatic islet cell growth.

In a further aspect is provided a method of treating diabetes comprising administering to a subject an HGF-MET agonist. In a preferred embodiment, the method is characterised by inducing increased pancreatic islet cell growth.

In a further aspect is provided an HGF-MET agonist for use in a method provided herein.

In a further aspect is provided a pharmaceutical composition for use in a method provided, wherein the pharmaceutical composition comprises an HGF-MET agonist and a pharmaceutically acceptable excipient or carrier.

In preferred embodiments of all aspects, the HGF-MET agonist is an anti-MET agonist antibody.

### DETAILED DESCRIPTION OF THE INVENTION

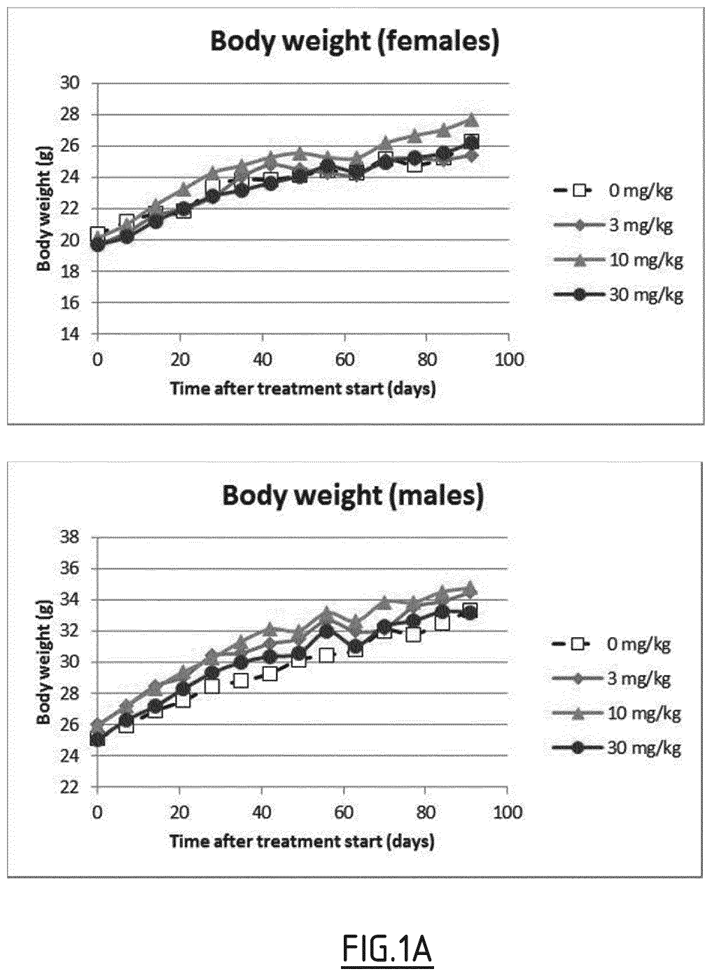

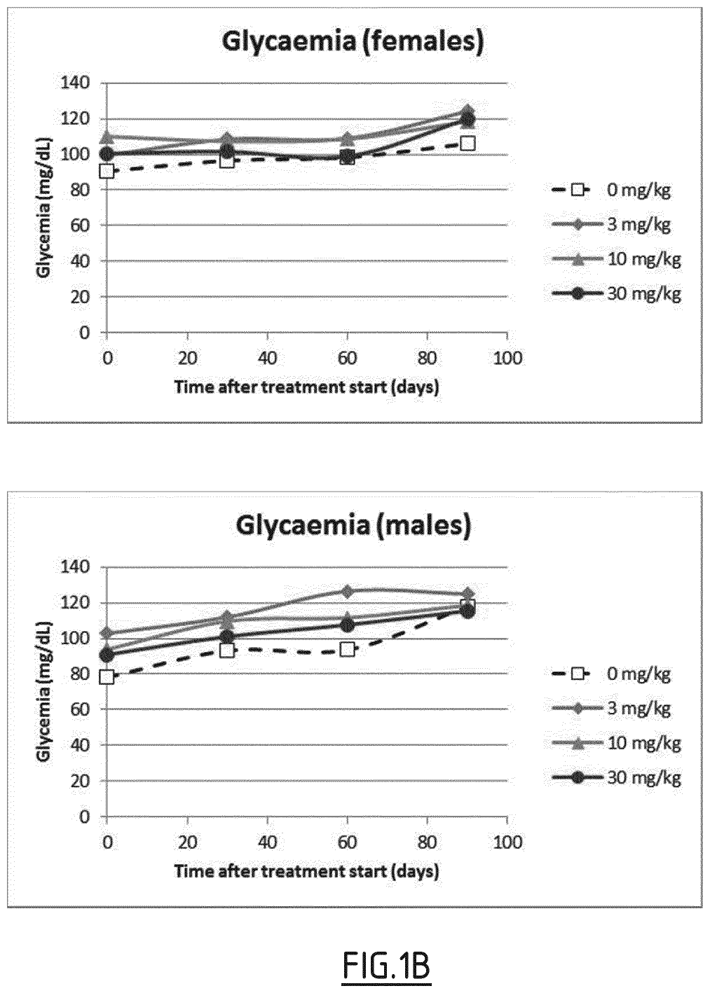

FIG. 1. MET agonist antibody treatment does not alter basic metabolism in healthy mice. In order to assess the biological effect of a MET agonistic antibody on Langerhans islet cells in vivo, we subjected both male and female adult BALB/c mice to systemic treatment with 0, 3, 10 or 30 mg/kg purified 71D6 antibody for a period of three months (6 mice per gender per group for a total of 48 animals). Antibody was administered 2 times a week by i.p. injection. Body weight and fasting blood glucose concentration was measured every month throughout the experiment. (A) Body weight over time. (B) Basal glycemia over time.

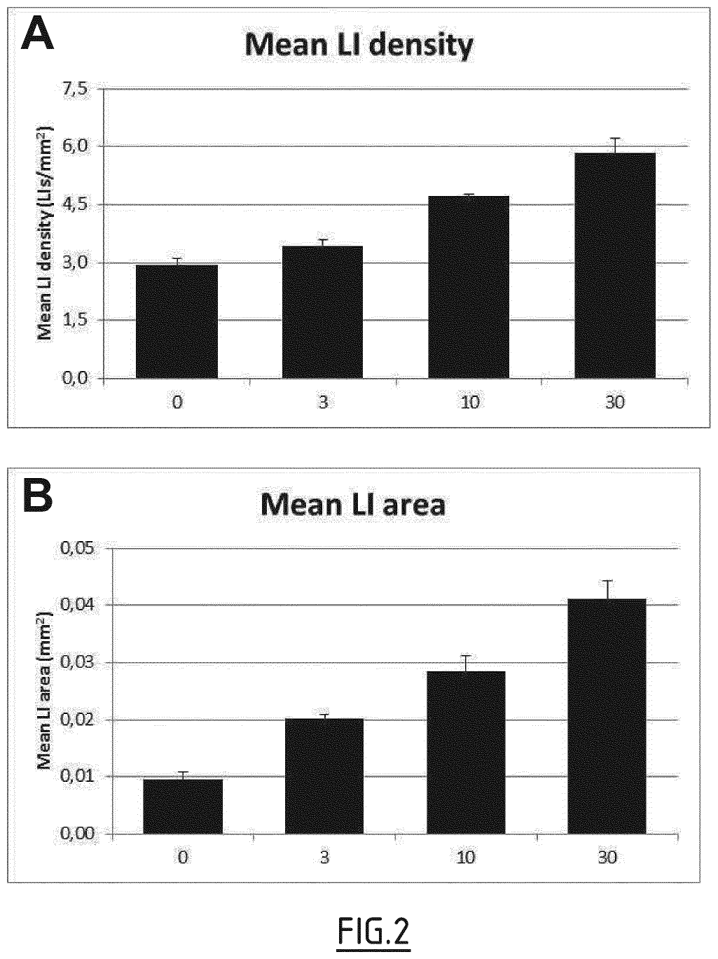

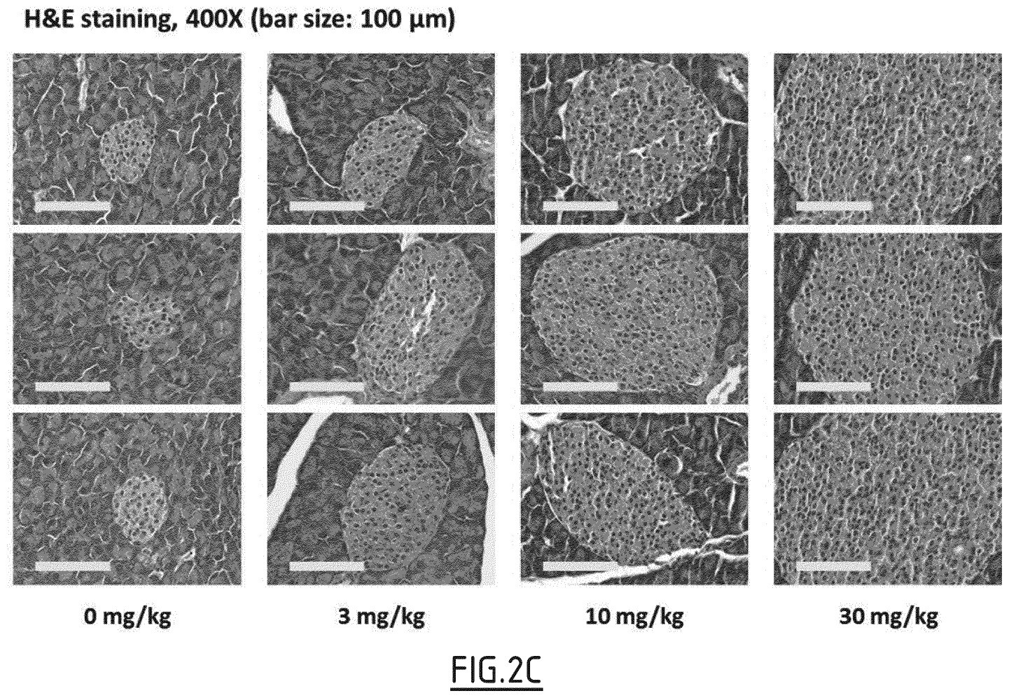

FIG. 2. MET agonist antibody treatment promotes Langerhans islet growth in healthy mice. Adult BALB-c mice were subjected to chronic treatment with increasing concentration of the 71D6 MET agonist antibody as described in FIG. 1 legend. At the end of the experiment, mice were sacrificed and subjected to autopsy. Pancreases were extracted, processed for histological analysis and embedded in paraffin. Sections were stained with hematoxylin and eosin, examined by microscopy and photographed. Images were analysed using ImageJ software to determine Langerhans islet number and size. (A) Mean Langerhans islet density. (B) Mean Langerhans islet size. (C) Representative images of pancreas sections stained with hematoxylin and eosin. Magnification: 400×.

FIG. 3. MET agonist antibody treatment promotes Langerhans islet cell growth in healthy mice. Adult BALB-c mice were subjected to chronic treatment with increasing concentration of the 71D6 MET agonist antibody as described above. Pancreas sections were analysed by immunohistochemistry using anti-insulin antibodies. The Figure shows representative images taken at the microscope at a 100× magnification.

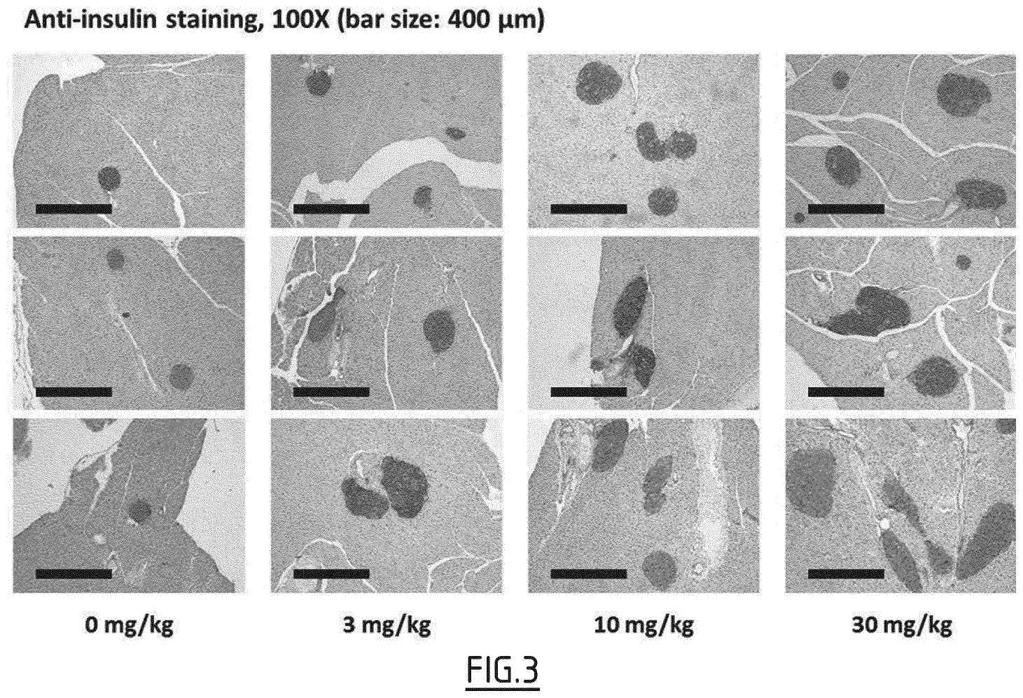

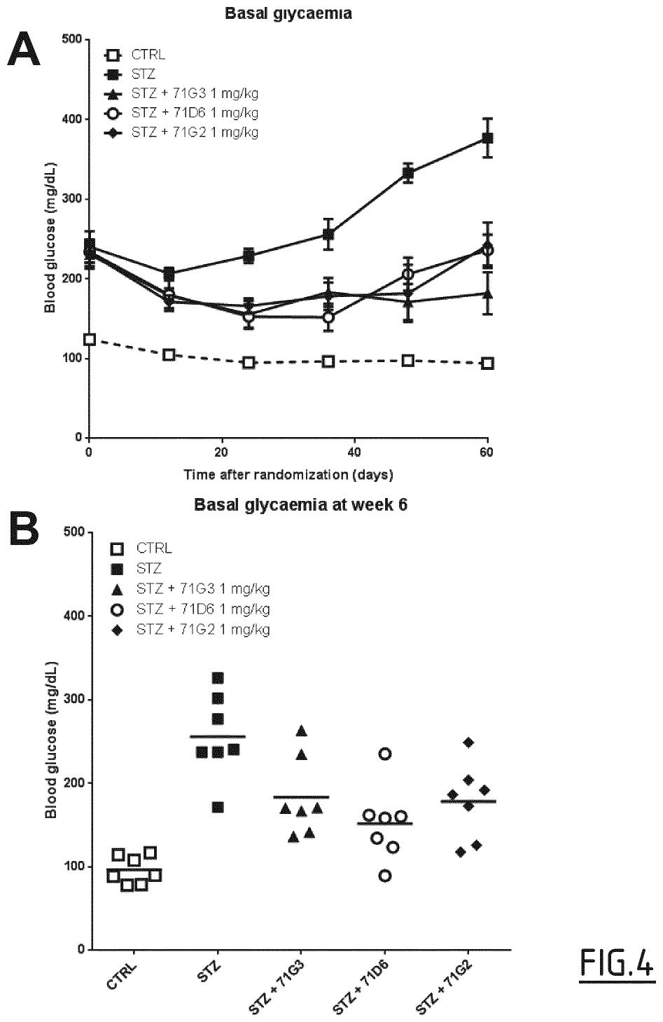

FIG. 4. MET agonist antibodies normalize basal glycaemia in a mouse model of type 1 diabetes mellitus. Streptozotocin (STZ), a chemical agent that selectively kills beta cells and a standard compound used to induce type 1 diabetes mellitus in laboratory animals, was injected i.p. into female BALB-c mice at a dose of 40 mg/kg every 24 hours for 5 consecutive days. One week after the last injection, STZ-treated mice were randomized into 4 arms of 7 mice each based on basal glycemia, which received treatment with (i) vehicle only (STZ), (ii) purified 71D6 antibody (STZ+71D6), (iii) purified 71G2 antibody (STZ+71G2), (iv) purified 71G3 antibody (STZ+71G3). Antibodies were administered by i.p. injection at a dose of 1 mg/kg two times a week for 8 weeks. An additional, fifth arm contained 7 mice that received no STZ or antibody and served as a healthy control (CTRL). Basal glycemia was monitored throughout the experiment. (A) Basal glycaemia over time. (B) Basal glycemia at week 6 of treatment.

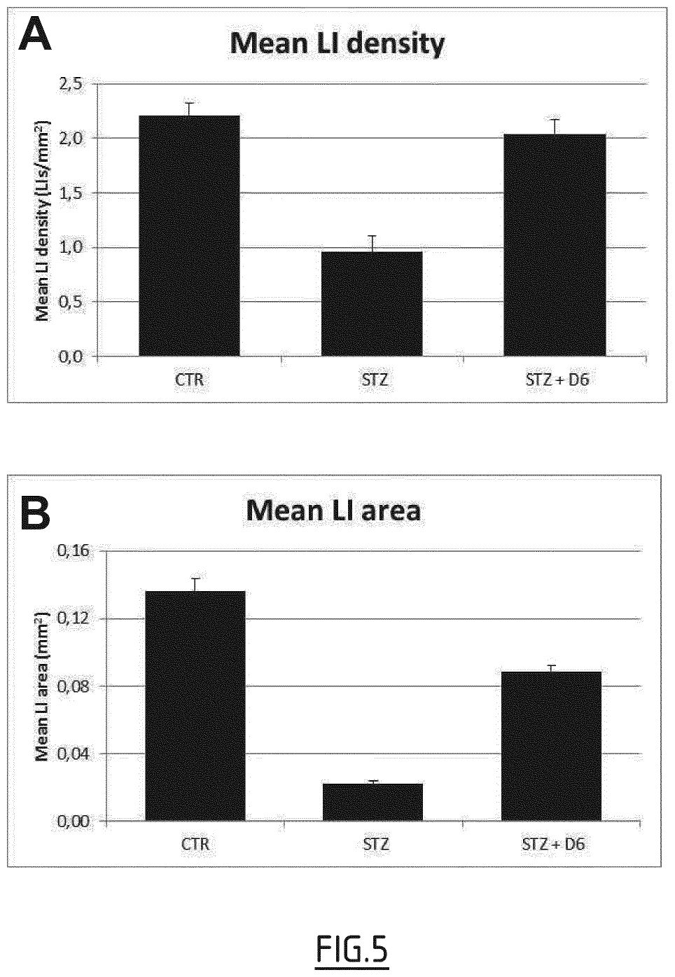

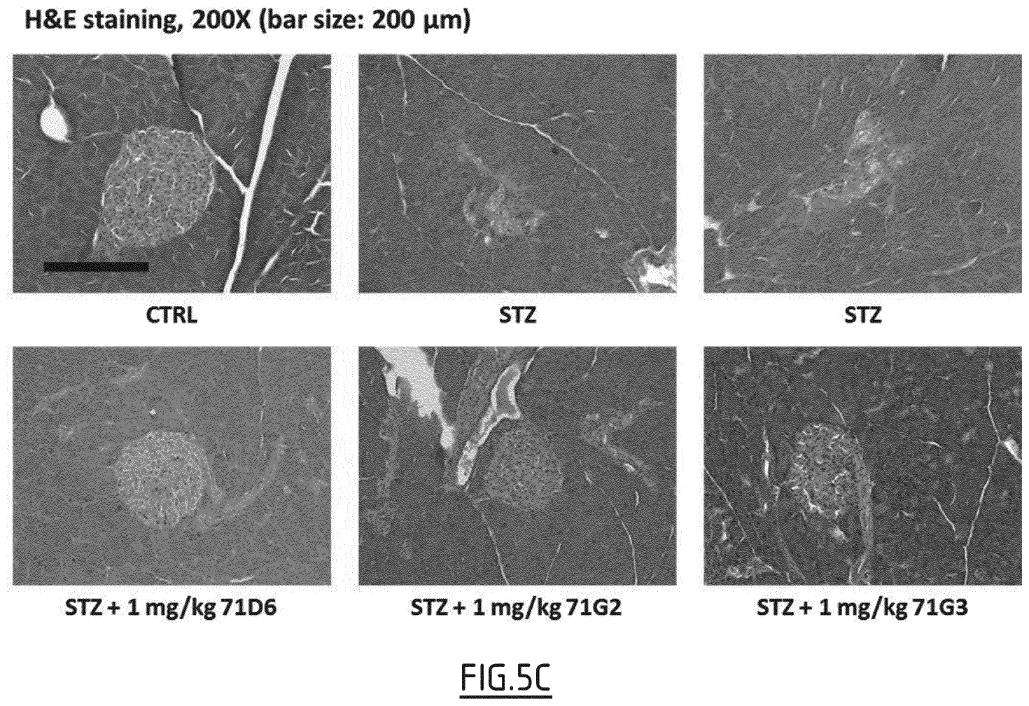

FIG. 5. MET agonist antibodies promote Langerhans islet regeneration in a mouse model of type 1 diabetes mellitus. STZ-injected BALB-c mice were treated with 1 mg/kg 71D6, 71G2 or 71G3 as described in FIG. 4 legend. After 8 weeks of antibody treatment, mice were sacrificed and subjected to autopsy. Pancreas sections were stained with hematoxylin and eosin, analysed by microscopy and photographed. Digital images of Langerhans islets were analysed using ImageJ software. The number, density and size of Langerhans islets were determined by digital data analysis. (A) Mean Langerhans islet density. (B) Mean Langerhans islet size. (C) Representative images of pancreas sections stained with hematoxylin and eosin. Magnification: 200×.

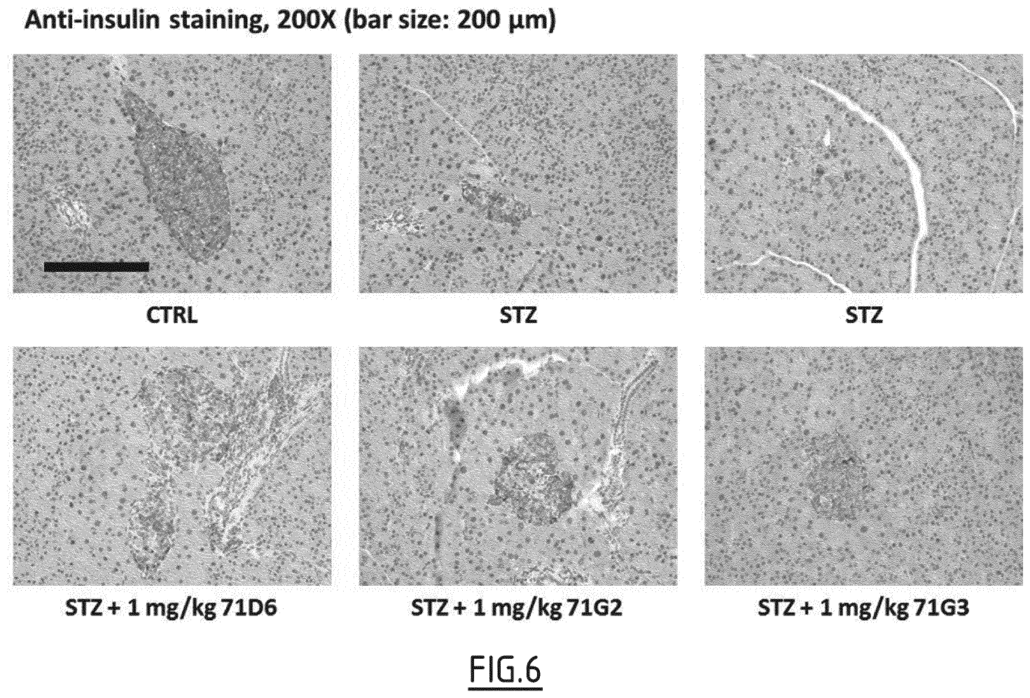

FIG. 6. MET agonist antibodies promote pancreatic islet cell regeneration in a mouse model of type 1 diabetes mellitus. STZ-injected BALB-c mice were treated with 1 mg/kg 71D6, 71G2 or 71G3 as described above. Pancreas sections were analysed by immunohistochemistry using anti-insulin antibodies. The Figure shows representative images taken at the microscope at a 200× magnification.

FIG. 7. MET agonist antibodies normalize basal glycaemia in a mouse model of type 2 diabetes mellitus. Female db/db mice were randomized into 4 arms of five mice each, which received treatment with (i) vehicle only (PBS), (ii) purified 71D6 antibody, (iii) purified 71G2 antibody, (iv) purified 71G3 antibody. Antibodies were administered by i.p. injection at a dose of 1 mg/kg two times a week for 8 weeks. C57BL6/J mice were used as non-diabetic control animals. Basal glycemia was monitored throughout the experiment. (A) Basal glycaemia over time. (B) Basal glycemia at week 8 of treatment.

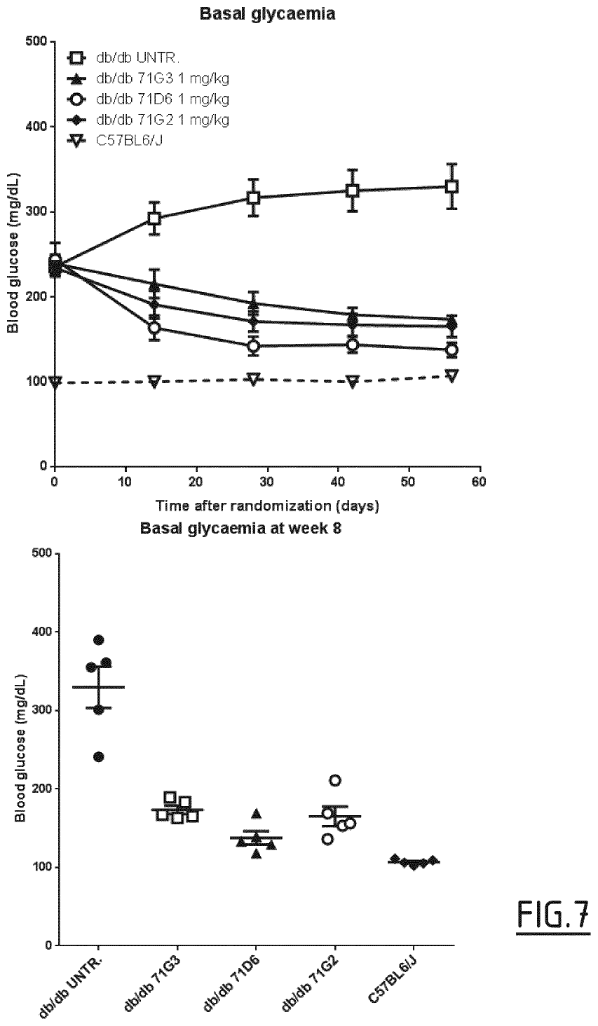

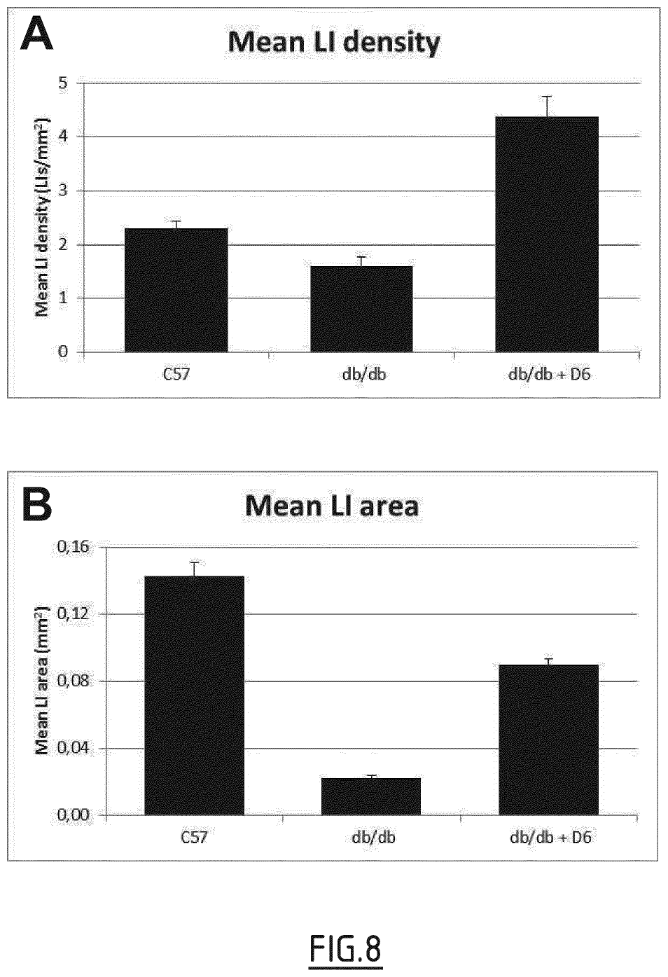

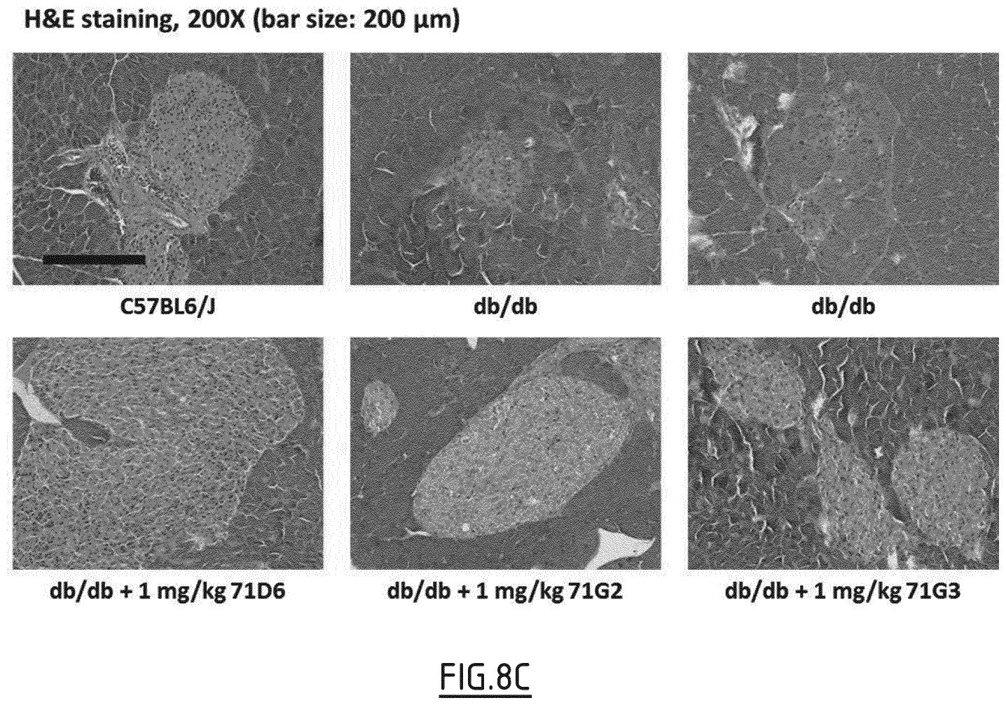

FIG. 8. MET agonist antibodies promote Langerhans islet regeneration in a mouse model of type 2 diabetes mellitus. Female db/db mice were treated with 71D6, 71G2 and 71G3 as described in FIG. 7 legend. After 8 weeks of treatment, mice were sacrificed and subjected to autopsy. Pancreases were collected, processed for histology and embedded in paraffin. Tissues sections were stained with hematoxylin and eosin, analysed by microscopy, and photographed. Langerhans islets were analysed using ImageJ software to estimate islet number, density and size. (A) Mean Langerhans islet density. (B) Mean Langerhans islet size. (C) Representative images of pancreas sections stained with hematoxylin and eosin. Magnification: 200×.

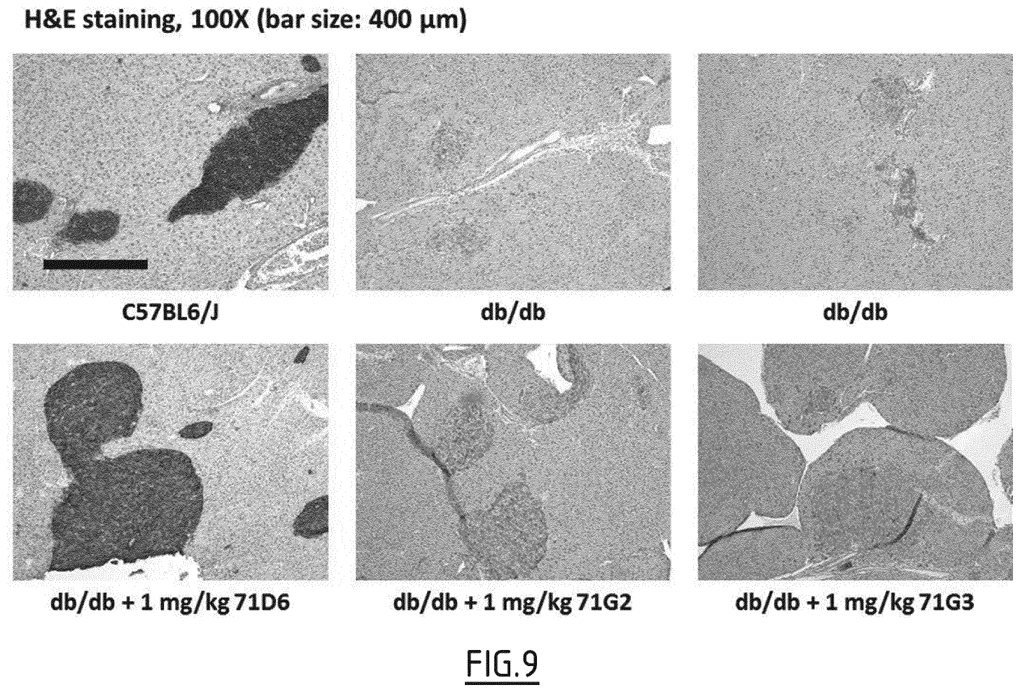

FIG. 9. MET agonist antibodies promote pancreatic islet cell regeneration in a mouse model of type 2 diabetes mellitus. Female db/db mice were treated with 71D6, 71G2 and 71G3 as described above. Pancreas sections were analysed by immunohistochemistry using anti-insulin antibodies. The Figure shows representative images taken at the microscope at a 100× magnification.

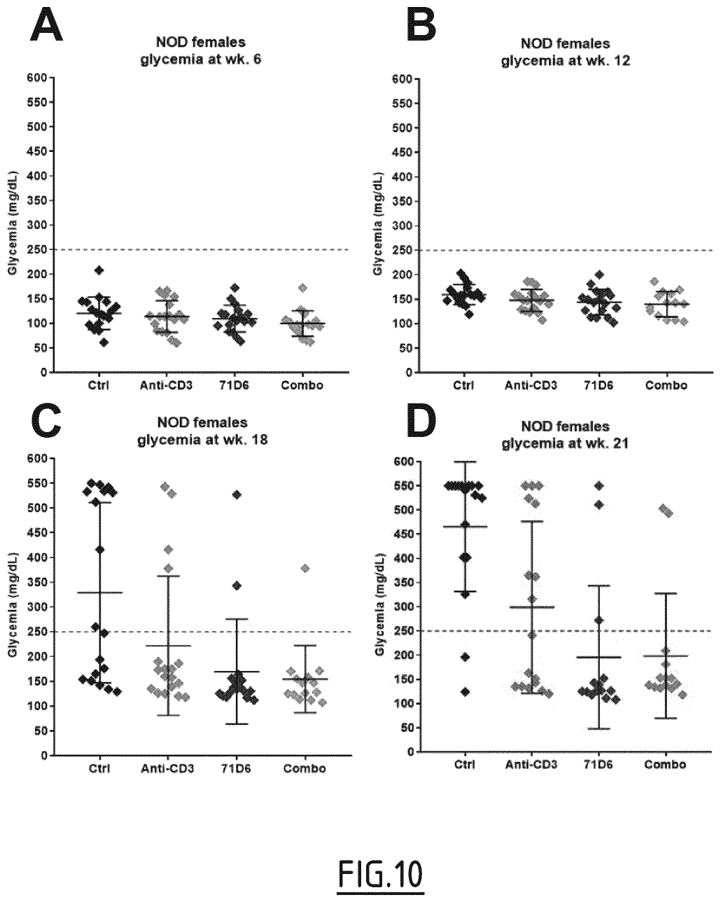

FIG. 10. Blood sugar content in NOD mice. Blood sugar was measured in random fed (i.e. not fasting) animals using test strips for human use (multiCare in; Biochemical Systems International). At week 6 of age, NOD mice displayed a pre-diabetic, average glycemia of approximately 110 mg/dL. Starting from week 7, animals were subjected to treatment as described in the text. Glycemia was monitored one time per week for the whole duration of the experiment. An animal was considered diabetic if it showed a glycemic value greater than 250 mg/dL (horizontal dotted lines) for 2 consecutive weeks.

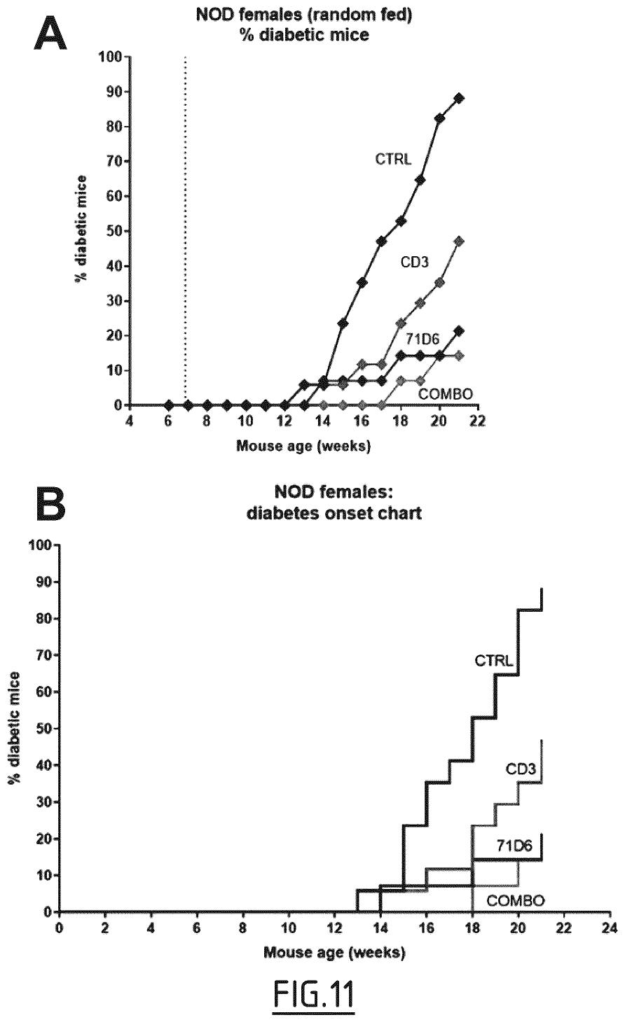

FIG. 11. Analysis of diabetes onset. (A) Percentage of diabetic mice over time. The vertical dotted line indicates the time of treatment start. (B) Kaplan-Meier analysis of diabetes onset. Statistical analysis was performed using Prism software (Graph Pad). A Mantel-Cox test, a Logrank test for trend and a Gehan-Breslow-Wilcoxon test all gave a p value of less than 0.001, indicating that the differences among curves are statistically significant.

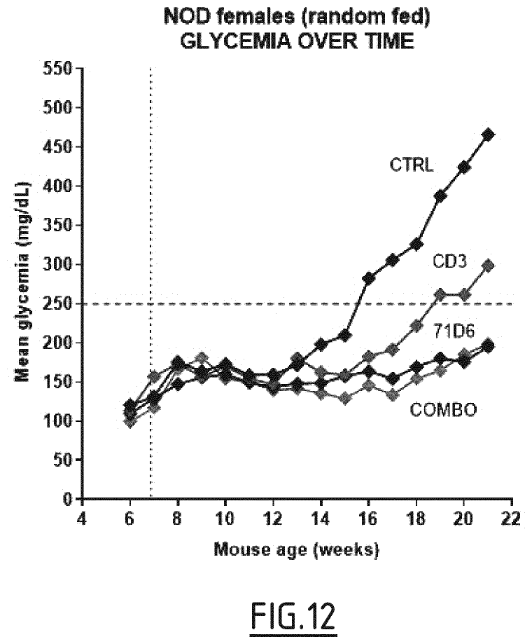

FIG. 12. Analysis of non-fasting glycemia over time. Glycemia was measured in random fed (i.e. not fasting) animals as described above one time per week. Consistent with the diabetes onset data, blood sugar levels followed a precise order: CONTROL>CD3>71D6>COMBO.

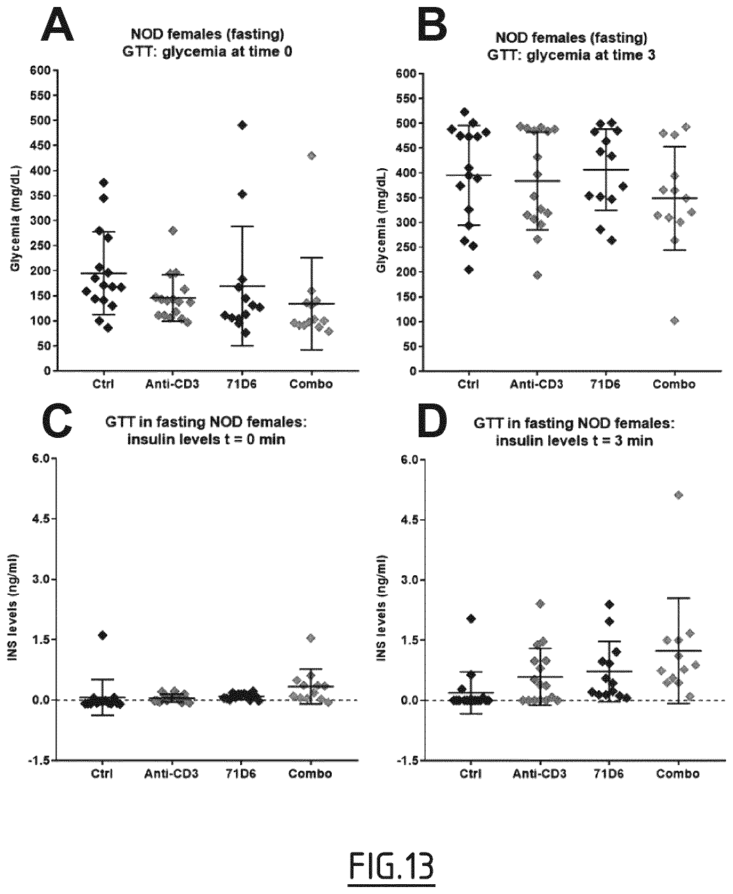

FIG. 13. Glucose tolerance test (GTT). Before sacrifice, all mice were subjected to a glucose tolerance test (GTT). To this end, animals were food-starved overnight. The morning after, a blood sample was collected for glycemia and insulin measurement. A glucose solution (3 g/kg in 200 μL PBS) was injected i.p. and a second blood sample was collected 3 minutes later. Blood glucose concentration was determined using strips as described above. Insulin concentration was measured with an Ultra-Sensitive Mouse Insulin ELISA Kit (Crystal Chem). (A) Glycemia at time zero. (B) Glycemia at time 3 minutes. (C) Insulin levels at time zero. (D) Insulin levels at time 3 minutes.

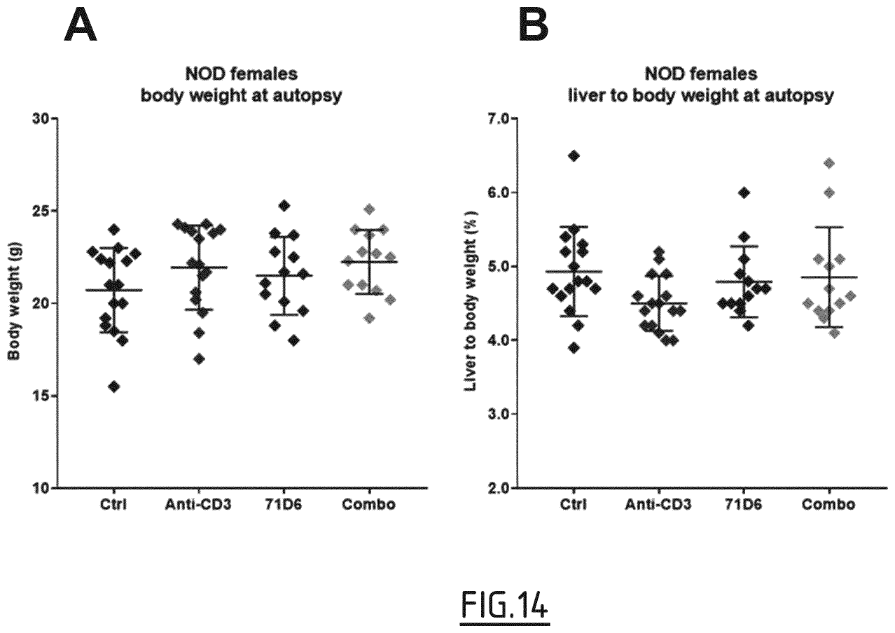

FIG. 14. Body weight and liver to body weight at autopsy. (A) Body weight. Consistent with an ameliorated diabetic phenotype, body weight was slightly (although not significantly) higher in the treatment arms compared to the control arm. (B) Liver to body weight. There was no significant difference in liver to body weight in any of the group, suggesting that 71D6-mediated liver growth (observed in other mouse systems) is strain-specific.

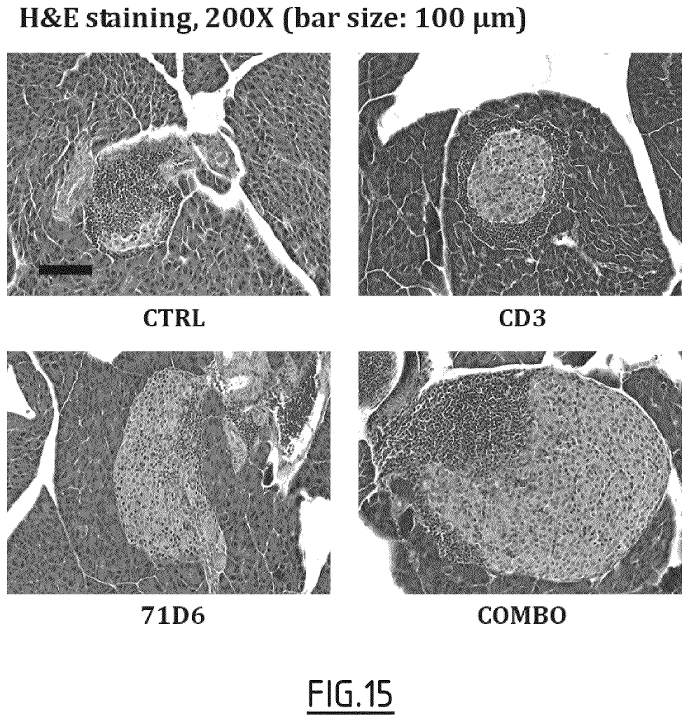

FIG. 15. Histological analysis of pancreas sections. Pancreas samples were embedded in paraffin and processed for histological analysis. Tissue section were stained with hematoxylin and eosin (H&E) and analyzed by microscopy. Representative images of each treatment arm are shown. Magnification: 200×.

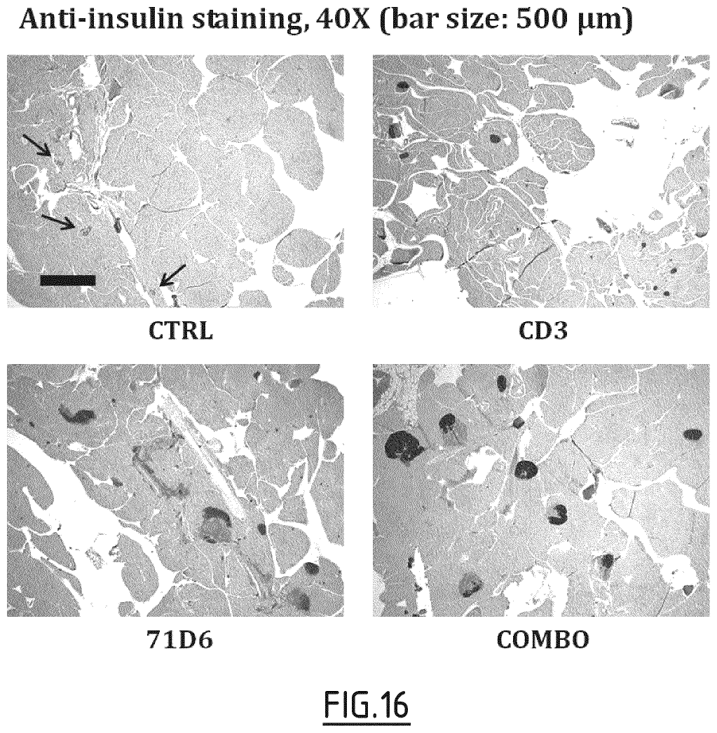

FIG. 16. Immuno-histochemical analysis of insulin expression. Pancreas sections were stained with anti-insulin antibodies and analyzed by microscopy. Representative images of each treatment arm are shown. Magnification: 40×.

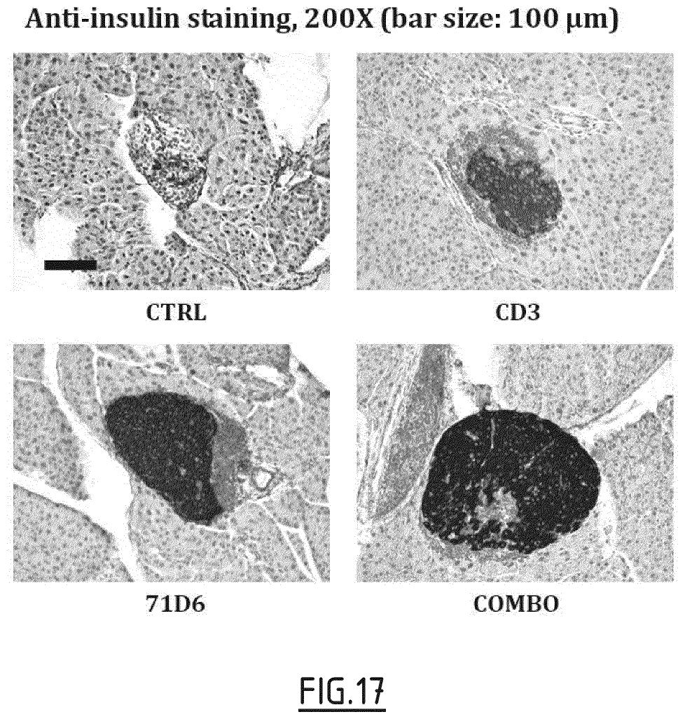

FIG. 17. High power microscopical analysis of insulin expression. Pancreas sections were stained with anti-insulin antibodies as above. Representative microscope images of each treatment arm are shown. Magnification: 200×.

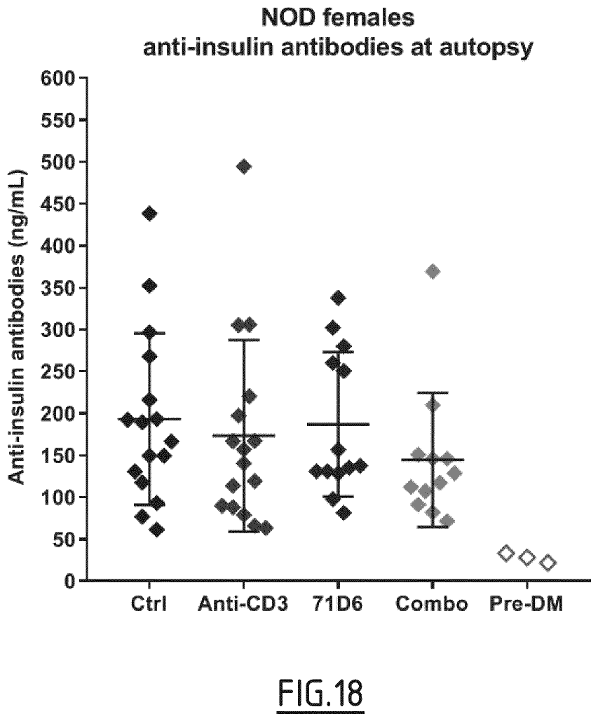

FIG. 18. Anti-insulin auto-antibodies in mouse plasma. Plasma samples collected at autopsy from all mice as well as from young, pre-diabetic female NOD mice (week 7 of life) were analyzed using a Mouse IAA (Insulin Auto-Antibodies) ELISA Kit (Fine Test). This analysis revealed that most mice displayed high concentrations of anti-insulin antibodies compared to pre-diabetic animals (last group on the right). While no statistically significant difference was observed among the different populations, mice of the COMBO arm displayed a trend towards lower levels. Mice of the 71D6 arm could be clearly divided into 2 subpopulations with low and high auto-antibodies levels, respectively. While these results warrant further investigation, they overall strengthen the hypothesis that neither anti-CD3 antibodies nor 71D6 treatment affect the production of auto-antibodies in this system, but rather act downstream to prevent or delay the onset of diabetes.

### DETAILED DESCRIPTION

As used herein, “pancreatic islet cell” is used to refer to those islet cells of the pancreas also known as “islets of Langerhans”, and include alpha, beta, and delta islet cells, plus islet stroma. Means of identifying pancreatic islet cells are known to the skilled person, for example histological examination of cell biopsies.

Promotion of islet cell growth as used herein can refer to an increase in the growth of pancreatic islet cells in a subject that has received an HGF-MET agonist compared to in that subject prior to intervention. Similarly, promotion of islet cell growth can refer to an increase of pancreatic islet cells in a subject that has received an HGF-MET agonist compared to a comparable control subject that has not received an HGF-MET agonist. Pancreatic islet cell growth can be characterised by an increase in the density of islets (number per mm²), an increase in the islet size (e.g. area), or both an increase in islet density and islet size.

Promotion of beta islet cell growth as used herein can refer to an increase in the growth of beta islet cells in a subject that has received an HGF-MET agonist compared to in that subject prior to intervention. Similarly, promotion of beta islet cell growth can refer to an increase of pancreatic islet cells in a subject that has received an HGF-MET agonist compared to a comparable control subject that has not received an HGF-MET agonist. Pancreatic islet cell growth can be characterised by an increase in the density of islets (number per mm²), an increase in the islet size (e.g. area), or both an increase in islet density and islet size.

Promotion of insulin production as used herein can refer to an increase in the insulin production by (beta) islet cells in a subject that has received an HGF-MET agonist compared to in that subject prior to intervention. Similarly, promotion of insulin production can refer to an increase of insulin production by (beta) islet cells in a subject that has received an HGF-MET agonist compared to a comparable control subject that has not received an HGF-MET agonist. Insulin production can be characterised by one or more of an increase in plasma insulin levels, an increase in beta cell density, an increase in beta cell area, an increase in density and/or number of insulin-positive islet cells, or any combination of these measures.

A pancreatic tissue transplant, as used herein, refers to the transplant of any pancreatic tissue into a subject. The transplant may be a whole organ transplant—i.e. a whole pancreas transplant—or a partial pancreas transplant. The transplant may be a transplant of pancreatic islets or islet cells, also referred to herein as an pancreatic islet graft.

As used herein, “HGF-MET agonist” and “MET agonist” are used interchangeably to refer to non-native agents that promote signalling via the MET protein—i.e. agents other than HGF that bind MET and increase MET signalling. Agonist activity on binding of MET by MET agonists is indicated by molecular and/or cellular responses that (at least partially) mimic the molecular and cellular responses induced upon HGF-MET binding. Suitable methods for measuring MET agonist activity are described herein, including in the Examples. A “full agonist” is a MET agonist that increases MET signalling in response to binding to an extent at least similar, and optionally exceeding, the extent to which MET signalling increases in response to binding of the native HGF ligand. Examples of the level of MET signalling induced by “full agonists”, as measured by different methods of determining MET signalling, are provided herein.

Immunosuppressive agents, also referred to as immunosuppressants, as used herein refer to therapeutic agents intended to reduce or inhibit an immune response in a subject, for example anti-inflammatory agents and tolerising agents. Examples of immunosuppressants include check-point inhibitors (e.g. PD-L1 molecules, CTLA4 molecules (e.g. abatecept)), TNF inhibitors (e.g. anti-TNF antibodies, etanercept), tolerising dendritic cells, anti-CD3 antibodies, anti-inflammatory cytokines (e.g. IL-10).

HGF-MET agonists may be small molecules, binding proteins such as antibodies or antigen binding fragments, aptamers or fusion proteins. A particular example of a MET agonist is an anti-MET agonist antibody.

As used herein, “treatment” or “treating” refers to effective therapy of the relevant condition—that is, an improvement in the health of the subject. Treatment may be therapeutic or prophylactic treatment—that is, therapeutic treatment of subjects suffering from the condition, or prophylactic treatment of a subject so as to reduce their risk of contracting the condition or the severity of the condition once contracted. Therapeutic treatment may be characterised by improvement in the health of the subject compared to prior to treatment. Therapeutic treatment may be characterised by improvement in the health of the subject compared to a comparable control subject that has not received treatment. Therapeutic treatment may also be characterised by stabilisation of the health of the subject compared to prior to treatment, i.e. inhibition of progression of a disease state in the subject. Prophylactic treatment may be characterised by improvement in the health of the subject compared to a control subject (or population of control subjects) that has not been treated.

As used herein, the term “antibody” includes an immunoglobulin having a combination of two heavy and two light chains which have significant specific immuno-reactive activity to an antigen of interest (e.g. human MET). The terms “anti-MET antibodies” or “MET antibodies” are used interchangeably herein to refer to antibodies which exhibit immunological specificity for human MET protein. “Specificity” for human MET does not exclude cross-reaction with species homologues of MET. In particular, “agomAbs” as used herein refers MET antibodies that bind to both human MET and mouse MET.

“Antibody” as used herein encompasses antibodies of any human class (e.g. IgG, IgM, IgA, IgD, IgE) as well as subclasses/isotypes thereof (e.g. IgG1, IgG2, IgG3, IgG4, IgA1). Antibody as used herein also refers to modified antibodies. Modified antibodies include synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. In addition, the term “modified antibody” includes multivalent forms of antibodies (e.g., trivalent, tetravalent, etc., antibodies that bind to three or more copies of the same antigen).

Antibodies described herein may possess antibody effector function, for example one or more of antibody dependent cell-mediated cytotoxicity (ADCC), complement dependent cytotoxicity (CDC) and antibody dependent cellular phagocytosis (ADCP). Alternatively, in certain embodiments antibodies for use according to the invention have an Fc region that has been modified such that one or more effector functions, for example all effector functions, are abrogated.

Antibodies comprise light and heavy chains, with or without an interchain covalent linkage between them. An antigen-binding fragment of an antibody includes peptide fragments that exhibit specific immuno-reactive activity to the same antigen as the antibody (e.g. MET). Examples of antigen-binding fragments include scFv fragments, Fab fragments, and F(ab′)2 fragments.

As used herein, the terms “variable region” and “variable domain” are used interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20, 267-279, 2000). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20, 267-279, 2000). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20, 267-279, 2000).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, Md., 1991) and the limits of the HVs and the CDRs may be different in some VH and VL domains.

The CDRs of the VL and VH domains can typically be defined as comprising the following amino acids: residues 24-34 (CDRL1), 50-56 (CDRL2) and 89-97 (CDRL3) in the light chain variable domain, and residues 31-35 or 31-35b (CDRH1), 50-65 (CDRH2) and 95-102 (CDRH3) in the heavy chain variable domain; (Kabat et al., Sequences of Proteins of Immunological Interest, 5th Ed. Public Health Service, National Institutes of Health, Bethesda, Md., 1991). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227, 799-817, 1992; Tramontano et al., J. Mol. Biol, 215, 175-182, 1990). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “CDR” or “complementarity determining region” means the non-contiguous antigen combining sites found within the variable region of both heavy and light chain polypeptides. These particular regions have been described by Kabat et al., J. Biol. Chem. 252, 6609-6616, 1977, by Kabat et al., Sequences of Proteins of Immunological Interest, 5th Ed. Public Health Service, National Institutes of Health, Bethesda, Md., 1991, by Chothia et al., J. Mol. Biol. 196, 901-917, 1987, and by MacCallum et al., J. Mol. Biol. 262, 732-745, 1996, where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions.

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework region” or “FR region” includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by interchain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161, 4083-4090, 1998). MET antibodies comprising a “fully human” hinge region may contain one of the hinge region sequences shown in Table 2 below.

TABLE 2
Human hinge sequences.

| IgG | Upper hinge | Middle hinge | Lower hinge |
|---|---|---|---|
| IgG1 | EPKSCDKTHT | CPPCP | APELLGGP |
| | (SEQ ID NO: 199) | (SEQ ID NO: 200) | (SEQ ID NO: 201) |
| IgG3 | ELKTPLGDTTHT | CPRCP | APELLGGP |
| | (SEQ ID NO: 202) | (EPKSCDTPPPCPRCP)₃ | (SEQ ID NO: 204) |
| | | (SEQ ID NO: 203) | |
| IgG4 | ESKYGPP | CPSCP | APEFLGGP |
| | (SEQ ID NO: 205) | (SEQ ID NO: 206) | (SEQ ID NO: 207) |
| IgG42 | ERK | CCVECPPPCP | APPVAGP |
| | (SEQ ID NO: 208) | (SEQ ID NO: 209) | (SEQ ID NO: 210) |

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; Kabat et al., Sequences of Proteins of Immunological Interest, 5th Ed. Public Health Service, National Institutes of Health, Bethesda, Md. (1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “fragment” refers to a part or portion of an antibody or antibody chain comprising fewer amino acid residues than an intact or complete antibody or antibody chain. The term “antigen-binding fragment” refers to a polypeptide fragment of an immunoglobulin or antibody that binds antigen or competes with intact antibody (i.e., with the intact antibody from which they were derived) for antigen binding (i.e., specific binding to MET). As used herein, the term “fragment” of an antibody molecule includes antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

As used herein, “subject” and “patient” are used interchangeably to refer to a human individual. A “control subject” refers to a comparable subject that has not received the intervention.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Therapeutic Methods

It is demonstrated herein that HGF-MET agonists (especially MET agonist antibodies) promote growth of pancreatic islet cells in healthy subjects. It is also demonstrated that MET agonists (especially MET agonist antibodies) protect pancreatic islet cells from degeneration in subjects experiencing islet cell depletion or damage. Moreover, not only do HGF-MET agonists (especially MET agonist antibodies) protect islet cells in these subjects, but they promote growth and regeneration of new islet cells in subjects with depleted or degenerated pancreatic islet cell populations. Furthermore, the new islet cells induced by MET agonist administration are highly functional, restoring insulin production.

Promoting islet cell growth is particularly advantageous, as it treats the underlying pathophysiology of conditions such as diabetes (especially type 1 diabetes, but also type 2 diabetes). Current treatment relies on passively managing the symptoms using diet and often insulin injections. These approaches do not address the underlying cause of the disease. Herein it is surprisingly identified that administration of an exogenous, non-native HGF-MET agonist effectively promotes growth and regeneration of pancreatic islet cells. Therefore administration of an HGF-MET agonist (especially a MET agonist antibody) represents a solution to the long felt medical need for a clinically relevant therapy that addresses the problem of pancreatic cell degradation.

Accordingly, in one aspect, provided herein is a method of promoting pancreatic islet cell growth comprising administering to a subject an HGF-MET agonist. Also provided is an HGF-MET agonist for use for promoting pancreatic islet cell growth in a subject, or the use of an HGF-MET agonist for the manufacture of a medicament for promoting pancreatic islet cell growth in a subject.

In a further aspect is provided a method of promoting insulin production in a subject in need thereof, comprising administering to a subject an HGF-MET agonist. In a preferred embodiment of this aspect, the method is characterised by inducing increased pancreatic islet cell growth. Also provided is an HGF-MET agonist for use for promoting insulin production in a subject, or the use of an HGF-MET agonist for the manufacture of a medicament for promoting insulin production in a subject.

In a further aspect is provided method of treating diabetes comprising administering to a subject an HGF-MET agonist. In a preferred embodiment of this aspect, the method is characterised by inducing increased pancreatic islet cell growth. Alternatively, or in addition, the method is further characterised by promoting insulin production. In a further aspect is provided an HGF-MET agonist (for example a MET agonist antibody) for use in a method of treating diabetes, wherein the HGF-MET agonist promotes pancreatic islet cell growth. In still a further aspect is provided an HGF-MET agonist for use in a method of treating diabetes, wherein the HGF-MET agonist promotes insulin production. Also provided is an HGF-MET agonist for use for treating diabetes in a subject, or the use of an HGF-MET agonist for the manufacture of a medicament for treating diabetes in a subject.

As demonstrated herein, HGF-MET agonists (in particular MET agonist antibodies) promote pancreatic islet cell growth. This growth is characterised both by an increase in pancreatic islet cell area, as well as an increase in the density of islets in pancreatic tissue.

Accordingly, in a preferred embodiment of all methods provided herein, the method increases pancreatic islet cell density. In a preferred embodiment of all methods provided herein, the method increases pancreatic islet cell area.

It is demonstrated herein that HGF-MET agonists (e.g. MET agonist antibodies) promote growth of all pancreatic islet cells—that is, alpha, beta, gamma, delta and epsilon cells. Accordingly, in certain embodiments of all methods provided herein, the method promotes growth of any one or more of: alpha cells, beta cells, gamma cells, delta cells and epsilon cells. In certain embodiments, the method promotes growth of alpha cells. In certain embodiments, the method promotes growth of beta cells. In certain embodiments, the method promotes growth of gamma cells. In certain embodiments, the method promotes growth of delta cells. In certain embodiments, the method promotes growth of epsilon cells.

It is further demonstrated herein that HGF-MET agonists (e.g. MET agonist antibodies) are particularly effective at promoting growth of beta islet cells. This is particularly advantageous, as beta cells are crucial for insulin production and effective glucose control, and are degraded in conditions such as diabetes. Not only do HGF-MET agonists (e.g. MET agonist antibodies) promote beta cell growth, but the new cells are highly functional and produce insulin.

Accordingly, in preferred embodiments of all methods provided herein, the method promotes beta islet cell growth. In a preferred embodiment, the method increases beta islet cell density. In a preferred embodiment, the method increases beta islet cell area. In a preferred embodiment, the method promotes growth of insulin-producing beta cells.

The methods described herein will also be particularly advantageous in subjects that receive a pancreatic tissue transplant. Pancreatic tissue transplant is a possible treatment in subjects (such as diabetic subjects) where the islet cells have been destroyed. Such transplants can be in the form of a whole pancreas transplant, partial transplant of portion of a pancreas, or graft of isolated islets. In all instances, methods provided herein will be particularly advantageous in patients receiving such transplants and grafts, since the methods will promote survival of the transplanted islets and also growth and expansion of those cells.

Accordingly, in embodiments of all methods provided herein, the method further comprises administering to the subject a pancreatic tissue transplant. In certain embodiments, the method further comprises administering to the subject a whole pancreas transplant. In certain embodiments, the method further comprises administering to the subject a partial pancreas transplant. In certain embodiments, the method further comprises administering to the subject a pancreatic islet graft. In all such embodiments, administration of the HGF-MET agonist (for example a MET agonist antibody) and administration of the transplant can be performed in any order, or simultaneously.

In a further aspect is provided a method of improving pancreatic tissue transplant in a subject in need thereof, the method comprising administering to the subject an HGF-MET agonist. Also provided is an HGF-MET agonist for improving pancreatic tissue transplant in a subject, or the use of an HGF-MET agonist for the manufacture of a medicament for improving pancreatic tissue transplant in a subject. By ‘improving pancreatic tissue transplant’ it is herein meant that graft survival following transplantation and following proliferation of engrafted cells or tissue are improved.

Administration of HGF-MET agonists (e.g. MET agonist antibodies) is particularly advantageous in a type 1 diabetes context. Type 1 diabetes is characterised by significant, and often complete, degradation of the subject's beta islet cells. As a result, the subject cannot produce insulin and therefore cannot manage their blood glucose properly. As demonstrated herein, administration of HGF-MET agonists (e.g. MET agonist antibodies) can promote pancreatic islet cells (especially beta cells) even in subjects with depleted islet cell populations. These new islet cells as a result of the methods provided herein are functional, producing insulin. Type 1 diabetic subjects will therefore benefit from methods provided herein.

Accordingly, in certain embodiments of all methods provided herein, the subject has type 1 diabetes.

Although characterized by different etiological mechanisms, type 2 diabetes also leads to Langerhans islet degeneration. For example, the insulin resistance characteristic of type 2 diabetes places demands on the subject's beta cells to produce more insulin, ultimately leading to exhaustion and degeneration of the pancreatic islet cells. Therefore, regeneration of pancreatic islet cells, especially beta cells, is also an unmet medical need for type 2 diabetes mellitus patients. As demonstrated herein, HGF-MET agonists (e.g. MET agonist antibodies) are able to promote islet cell growth in a model of type 2 diabetes, leading to increased numbers of beta cells, increased insulin production and therefore better glycaemic control.

Accordingly, in certain embodiments of all methods provided herein, the subject has type 2 diabetes.

In Vitro Methods

It is demonstrated herein that growth of pancreatic islet cells is promoted by HGF-MET agonists. As well as being an important effect in vivo, HGF-MET agonists (such as MET agonist antibodies) will be advantageously used for in vitro expansion of pancreatic islet cells. Promoting growth of islet cells in vitro is important, for example, in preparation for islet cell grafts. Pancreatic islets that have been isolated in preparation for grafting will have limited viability in vitro. Contacting the isolated islet cells with an HGF-MET agonist (e.g. an anti-MET agonist antibody) will prolong the survival of the isolated islet cells in vitro. As a result, the window for effective grafting will be prolonged, and a greater proportion of the grafted islets will be viable. Similarly, isolated islets that are to be grafted can be expanded using HGF-MET agonists according to the provided methods, and thereby increase the cell population available for grafting.

Accordingly, in a further aspect is provided an in vitro method for promoting growth of a cell population or tissue comprising pancreatic islet cells, the method comprising contacting the cell population or tissue with an HGF-MET agonist. In preferred embodiments, the HGF-MET agonist is a MET agonist antibody.

The invention also relates to an ex vivo method of preserving an islet cell or pancreas transplant which comprises contacting the islet cell or pancreas transplant with an HGF-MET agonist, preferably a MET agonist antibody.

Subject or Patient

As demonstrated herein, administration of MET agonists (for example a MET agonist antibody) promotes growth of functional pancreatic islet cells. Promoting growth of pancreatic islet cells is especially important for patients either recently diagnosed with diabetes, especially type 1 diabetes, or even in so called “pre-diabetes”.

Typically, type 1 diabetes symptoms become manifest at adolescence. However, when the pathology is diagnosed, the majority of the patient's pancreatic beta cells have been destroyed (greater than 50%, for example 70% or 80% destruction). Langerhans islet cell degeneration occurs rapidly, particularly at the time when clinical symptoms become apparent and a diagnosis of diabetes is most-commonly made—as a result, the time-window for effective therapeutic intervention is narrow. This is evidenced by the fact that treatment with immunosuppressive agents (to restrict pancreatic islet cell degeneration) is most effective soon after diagnosis, preferably within 6 weeks.

Accordingly, in certain embodiments of the methods provided herein, the subject has been diagnosed with diabetes and first administration of the MET agonist (e.g. MET agonist antibody) is within 6 weeks of diagnosis. Preferably the first administration is within 5 weeks, within 4 weeks or within 3 weeks of diagnosis.

In certain embodiments, the subject has “pre-diabetes”. In such embodiments, “pre-diabetes” can be defined according to the American Diabetes Association (ADA) thresholds for fasting plasma glucose (FPG), for oral glucose tolerance test (OGTT), or both FPG and OGTT thresholds.

According to the ADA definition, “pre-diabetes” can be characterised by impaired fasting glucose—that is, a FPG of at least 100 mg/dl (5.6 mmol/l), but less than 126 mg/dl (7.0 mmol/l). Pre-diabetes can also be characterised by impaired glucose tolerance—that is OGTT results of at least 140 mg/dl (7.8 mmol/l) but less than 200 mg/dl (11.1 mmol/l). Patients with a fasting glucose of 126 mg/dl (7.0 mmol/l) or greater have impaired fasting glucose to the extent that they are diagnosed with diabetes. Patients with an OGTT of 200 mg/dl (11.1 mmol/l) or greater have impaired glucose tolerance to the extent that they are diagnosed with diabetes

Promoting islet cell growth in subjects still exhibiting partial glucose control (for example subjects in early stages of diabetes, or “pre-diabetes”) is particularly advantageous because these subjects still have a population of functioning islet cells. Methods according to the invention can therefore prolong the period in which such patients have functioning pancreatic islet cells.

Accordingly, in certain embodiments, the methods provided herein are methods of treating pre-diabetes.

In certain embodiments of the methods provided herein, the subject exhibits a fasting glucose of greater than 5.6 mmol/l. In certain embodiments, the subject exhibits a fasting glucose of greater than 6.1 mmol/l. In certain embodiments, the subject exhibits a fasting glucose of greater than 5.6 mmol/l and less than 7.0 mmol/l. In certain embodiments, the subject exhibits a fasting glucose of greater than 6.1 mmol/l and less than 7.0 mmol/l. In certain embodiments, the subject exhibits a fasting glucose of 7.0 mmol/l or greater.

In certain embodiments of the methods provided herein, the subject exhibits a fasting glucose of greater than 100 mg/dl. In certain embodiments, the subject exhibits a fasting glucose of greater than 110 mg/dl. In certain embodiments, the subject exhibits a fasting glucose of greater than 100 mg/dl and less than 126 mg/dl. In certain embodiments, the subject exhibits a fasting glucose of greater than 110 mg/dl and less than 126 mg/dl. In certain embodiments, the subject exhibits a fasting glucose of 126 mg/dl or greater.

In certain embodiments of the methods provided herein, the subject exhibits an OGTT of greater than 7.8. mmol/l. In certain embodiments, the subject exhibits a fasting glucose of greater than 7.8 mmol/l and less than 11.1 mmol/l. In certain embodiments, the subject exhibits a fasting glucose of 11.1 mmol/l or greater.

In certain embodiments of the methods provided herein, the subject exhibits an OGTT of greater than 140 mg/dl. In certain embodiments, the subject exhibits a fasting glucose of greater than 140 mg/dl and less than 200 mg/dl. In certain embodiments, the subject exhibits a fasting glucose of 200 mg/dl or greater.

In certain embodiments of the methods provided herein, the subject is an adolescent—that is, the subject is 10-19 years of age, for example 12-18 years of age.

As already described, the methods provided herein are particularly advantageous for subjects that have depleted islet cell levels but still have a population of functioning islet cells. This is because the methods can promote the survival of the remaining islet cells and at the same time promote growth and regeneration of new islet cells.

Accordingly, in certain embodiments of all methods provided herein, the subject is characterised by having a population of pancreatic islet cells at least 50% smaller than a healthy individual. In certain embodiments, the subject has a population of pancreatic islet cells at least 70%, optionally at least 80%, at least 90%, or at least 95% smaller than a healthy individual. In certain embodiments, the subject has a population of pancreatic islet cells about 70% to about 80% smaller than a healthy individual.

Destruction of pancreatic islet cells by autoantibodies may occur for some time before clinical symptoms become evident and diabetes is diagnosed. During this period, auto-antibodies to islet cell antigens can be detected, indicating ongoing destruction of pancreatic islet cells. The methods provided herein are will be particularly advantageous in subjects in which such antibodies can be detected, especially if the subject is not yet symptomatic, because these subjects will still have a population of functioning islet cells that can be protected and regenerated using the methods.

Accordingly, in certain embodiments, the subject has autoantibodies to islet cell antigens detectable in their serum. In preferred such embodiments, the subject has not been diagnosed with diabetes. In certain embodiments, the method comprises the step of measuring the level of autoantibodies to islet cell antigens in the subject's serum and administering the MET agonist (e.g. MET agonist antibody) if the level is raised compared to the level characteristic of a healthy subject.

Subjects with latent autoimmune diabetes of adults (LADA) will particularly benefit from the methods provided herein. LADA is a form of diabetes in which progression is typically slower than diabetes diagnosed in juveniles. LADA can be characterised by impaired glycaemic control (e.g. hyperglycaemia) together with detection of C-peptide. Subjects may also have detectable antibodies against pancreatic islet cells. Degeneration of pancreatic islet cells (in particular beta islet cells) in LADA patients is slower. As a result, it is expected that these patients will retain a population of functioning islet cells for longer. The methods provided herein can promote the survival of the remaining islet cells and at the same time promote growth and regeneration of new islet cells, and will therefore particularly benefit LADA patients.

Accordingly, in certain embodiments, the subject has LADA. In certain embodiments, the method is a method of treating LADA.

The methods described herein will also be particularly advantageous in subjects that receive a pancreatic tissue transplant. Pancreatic tissue transplant is a possible treatment in subjects (such as diabetic subjects) where the islet cells have been destroyed. Such transplants can be in the form of a whole pancreas transplant, partial transplant of portion of a pancreas, or graft of isolated islets. In all instances, methods provided herein will be particularly advantageous in patients receiving such transplants and grafts, since the methods will promote survival of the transplanted islets and also growth and expansion of those cells.

Accordingly, in certain embodiments of all methods provided herein, the subject has previously received a pancreatic tissue transplant. In certain embodiments, the subject has previously received a whole pancreas transplant. In certain embodiments, the subject has previously received a partial pancreas transplant. In certain embodiments, the subject has previously received a pancreatic islet graft.

In preferred embodiments of all methods provided herein, the subject has type 1 diabetes. In preferred embodiments of all methods provided herein, the subject has type 2 diabetes.

As described elsewhere herein, the provided methods are particularly advantageous in a pancreatic tissue transplant context. In this context, the methods are particularly advantageous in promoting growth of the transplanted pancreatic islet cells. However, the methods are also advantageous when administered to a healthy subject from which pancreatic islet cells may be taken—i.e. a donor subject. As demonstrated herein, administration of an HGF-agonist (in particular a MET agonist antibody) to a healthy subject promotes growth of their pancreatic islet cells without adverse effects. Therefore, a healthy subject from which pancreatic tissue is going to be taken for transplant—i.e. a donor subject—will benefit from administration of an HGF-MET agonist (e.g. a MET agonist antibody) according to the provided methods, as doing so will promote growth of their pancreatic islet cells, thereby providing more cells for transplant. In addition, if the donor is a live donor, the remaining islet cell population will be larger following HGF-MET agonist administration.

Accordingly, in certain embodiments of the provided methods, the subject is a healthy donor subject.

In preferred embodiments of all aspects, the subject or patient is a mammal, preferably a human.

In preferred embodiments of all aspects, the subject is a subject in need of the method—i.e. the method is administered to a subject in need thereof.

Combination Therapies

HGF-MET agonists administered according to the methods provided herein are particularly advantageous when administered as a combination therapy with immunosuppressive therapies. This is because immunosuppressive agents can reduce the autoimmune-mediated islet cell destruction. However, repeated doses of immunosuppressive agents over a period of weeks and months can be required in order for this protection to take effect. During this lag period, the islet cells can continue to degenerate, often to the point that they are completely destroyed by the time the immunosuppressive takes clinical effect. Administration of an HGF-MET agonist according to the present invention can prolong the survival of islet cells. The treatment window for immunosuppressives to become effective is therefore lengthened, meaning the combination treatment is more likely to be effective at protecting the subject's islet cells. Moreover, as well prolonging survival of islet cells, the methods provided herein promote their growth. The combination therapy will therefore be more effective as a result of a longer effective treatment window for the immunosuppressive agent to reduce islet cell degradation alongside growth and expansion of new islet cells as a result of the MET agonist administration.

Accordingly, in certain embodiments of all methods and second medical indication uses provided herein, the subject is further administered one or more immunosuppressive agent. Accordingly, in certain embodiments, it is also provided an HGF-MET agonist for use in combination with one or more immunosuppressive agent for promoting pancreatic islet cell growth, for promoting insulin production, and/or for treating diabetes in a subject. Also provided is an HGF-MET agonist for use for promoting pancreatic islet cell growth, for promoting insulin production, and/or for treating diabetes in a subject who/which is undergoing therapy with one or more immunosuppressive agent.

The immunosuppressive agent will reduce autoimmune mediated degradation of islet cells. In certain embodiments, the one or more immunosuppressive agent is selected from the list consisting of: cyclosporin A; mycophenolate, vitamin D3, an anti-CD3 antibody, an anti-IL-21 antibody, an anti-CD20 antibody (e.g. rituximab), an anti-CTLA4 antibody, an anti-TNFα antibody (e.g. infliximab), an anti-IL1α antibody, an anti-IL1β antibody, anti-CD4 antibody, an anti-CD45 antibody, a CTLA4 molecule (e.g. abatacept), a TNFα inhibitor (e.g. etanercept), a PD-L1 molecule, an IL-1 receptor antagonist (e.g anakinra), pegylated granulocyte colony-stimulating factor (e.g. pegfilgrastim), human recombinant IFN-alpha, IL-10, Glutamic Acid Decarboxylase (GAD)-65, tolerising insulin peptides (e.g. insulin B:9-23, Proinsulin peptide 19-A3), DiaPep277 of HSP60, regulatory T cells (Tregs), and tolerising dendritic cells. For example, GAD-65 and IL-10 may be administered together, for instance as a transgenic bacteria (e.g. Lactococcus) expressing both molecules.

Combinations of administration of MET agonists (e.g. MET agonist antibodies) with immunosuppressive agents is particularly advantageous for subjects exhibiting early stage diabetes, or subjects exhibiting impaired glucose control. Particularly preferred patients or subjects are those described in the “Subject or Patient” section herein.

For example, it may be particularly advantageous in subjects exhibiting a fasting glucose level of greater than 5.6 mmol/l, for example greater than 5.6 mmol/l and less than 7.0 mmol/l. Although these patients have a proportion of their islet cells depleted, they still have a population of islet cells. By combining immunosuppressives and a MET agonist according to the methods provided herein, the remaining islet cell population can be protected from degradation and the growth of new islet cells promoted.

In certain embodiments, the methods and second medical indication uses provided herein are used in combination with an anti-diabetes medication. Examples of diabetes therapies include insulin, diet management, metformin, sulfonylureas, thiazolidinediones, dipeptidyl peptidase-4 inhibitors, SGLT2 inhibitors, and glucagon-like peptide-1 analogs. Accordingly, in certain embodiments, it is also provided an HGF-MET agonist for use in combination with an anti-diabetes medication for promoting pancreatic islet cell growth, for promoting insulin production, and/or for treating diabetes in a subject. Also provided is an HGF-MET agonist for use for promoting pancreatic islet cell growth, for promoting insulin production, and/or for treating diabetes in a subject who/which is undergoing therapy with an anti-diabetes medication.

Methods and second medical indication uses provided herein may further be advantageously combined with administration of insulin. Insulin therapy can manage the symptoms of a degraded islet cell population during the period in which the methods provided herein is expanding the islet cell population.

Accordingly, in certain embodiments of all aspects of the methods and second medical indication uses provided herein, the subject is administered insulin at least daily—that is, at least once per day, optionally more frequently.

Administration

It will be appreciated that, as used herein, administration of an HGF-MET agonist (for example an anti-MET agonist antibody) to a subject refers to administration of an effective amount of the agonist.

In certain embodiments, the HGF-MET agonist (for example anti-MET agonist antibody or antigen binding fragment thereof) is administered at a dose in the range of from about 0.1 mg/kg to about 40 mg/kg per dose. In certain embodiments, the HGF-MET agonist (for example anti-MET agonist antibody or antigen binding fragment thereof) is administered at a dose in the range of from 0.5 mg/kg to about 35 mg/kg, optionally from about 1 mg/kg to about 30 mg/kg. In certain preferred embodiments, the HGF-MET agonist (for example anti-MET agonist antibody or antigen binding fragment thereof) is administered at a dose in the range of from about 1 mg/kg to about 10 mg/kg. That is, a dose of about 1, 2, 3, 4, 5, 6, 7, 8, 9 or 10 mg/kg. In certain preferred embodiments, the HGF-MET agonist (for example anti-MET agonist antibody or antigen binding fragment thereof) is administered at a dose of 1 mg/kg, 3 mg/kg, 10 mg/kg or 30 mg/kg.

Suitable routes for administration of the HGF-MET agonist (for example an anti-MET agonist antibody) to a subject would be familiar to the skilled person. Preferably the MET agonist is administered parenterally. In certain preferred embodiments, the HGF-MET agonist is administered orally or per os (p.o.), subcutaneously (s.c.), intravenously (i.v.), intradermally (i.d.), intramuscularly (i.m.) or intraperitoneally (i.p.). In certain preferred embodiments, the HGF-MET agonist is a MET agonist antibody and is administered intravenously.

The HGF-MET agonist (for example anti-MET agonist antibody) can be administered according to a regimen that maintains an effective level of the agonist in the subject. The skilled person is familiar with suitable dosage regimens. For example, in certain embodiments, the HGF-MET agonist (e.g. MET agonist antibody) is administered according to a dosage regimen of at least once per week—that is, a dose is administered approximately every 7 days or more frequently. In certain embodiments, the HGF-MET agonist (e.g. MET agonist antibody) is administered 1-3 times a week (i.e. 1, 2 or 3 times a week). In certain preferred embodiments, the HGF-MET agonist (e.g. MET agonist antibody) is administered twice per week. In certain preferred embodiments, the HGF-MET agonist is a MET agonist antibody and is administered once per week or twice per week.

For the methods described herein, the HGF-MET agonist (e.g. MET agonist antibody) is administered for a period sufficient to achieve effective treatment. The skilled person is able to determine the necessary treatment period for any individual patient. In certain embodiments, the HGF-MET agonist (e.g. a MET agonist antibody) is administered for a treatment period of at least 1 week. In certain embodiments, the HGF-MET agonist (e.g. a MET agonist antibody) is administered for a treatment period of at least 2 weeks, at least 3 weeks, or at least 4 weeks. In certain embodiments, the HGF-MET agonist (e.g. a MET agonist antibody) is administered for a treatment period of at least 1 month, at least 2 months or at least 3 months. In certain preferred embodiments, the HGF-MET agonist is a MET agonist antibody and is administered for a treatment period of 3 months.

It will be appreciated that the HGF-MET agonist (e.g. a MET agonist antibody) may be administered according to any combination of the described doses, dosage regimens and treatment periods. For example, in certain embodiments, the HGF-MET agonist (e.g. a MET agonist antibody) may be administered according to a dosage regimen of twice per week, at a dose of from 1 mg/kg to 5 mg/kg, for a period of at least 3 months. Other embodiments of the methods explicitly include other combinations of the recited doses, dosage regimens and treatment periods.

HGF-MET Agonist

In all aspects of the invention, an HGF-MET agonist is to be administered to a subject or patient. “HGF-MET agonist” and “MET agonist” are used interchangeably to refer to non-native agents that promote signalling via the MET protein—i.e. agents other than HGF that bind MET and increase MET signalling. Such agents may be small molecules, binding proteins such as antibodies or antigen binding fragments, aptamers or fusion proteins. A particular example of a MET agonist is an anti-MET agonist antibody.

Agonist activity on binding of MET by the MET agonists described herein is indicated by molecular and/or cellular responses that (at least partially) mimic the molecular and cellular responses induced upon HGF-MET binding.

Methods for determining MET agonism according to the invention, for example by MET agonist antibodies and antigen binding fragments, would be familiar to the skilled person. For example, MET agonism may be indicated by molecular responses such as phosphorylation of the MET receptor and/or cellular responses, for example those detectable in a cell scattering assay, an anti-apoptosis assay and/or a branching morphogenesis assay.

MET agonism may be determined by the level of phosphorylation of the MET receptor upon binding. In this context, a MET agonist antibody or antigen binding fragment, for example, causes auto-phosphorylation of MET in the absence of receptor-ligand binding—that is, binding of the antibody or antigen binding fragment to MET results in phosphorylation of MET in the absence of HGF. Phosphorylation of MET may be determined by assays known in the art, for example Western Blotting or phospho-MET ELISA (as described in Basilico et al., J Clin Invest. 124, 3172-3186, 2014, incorporated herein by reference).

MET agonism may alternatively be measured by induction of HGF-like cellular responses. MET agonism can be measured using assays such as a cell scattering assay, an anti-apoptosis assay and/or a branching morphogenesis assay. In this context, a MET agonist, for example an antibody or antigen binding fragment, induces a response in cellular assays such as these that resembles (at least partially) the response observed following exposure to HGF.

For example, a MET agonist (for example a MET agonist antibody) may increase cell scattering in response to the antibody compared to cells exposed to a control antibody (e.g. IgG1).

By way of further example, a MET agonist (for example a MET agonist antibody) may exhibit a protective potency against drug-induced apoptosis with an EC50 of less than 32 nM. By way of further example, a MET agonist (for example a MET agonist antibody) may exhibit an Emax cellular viability of greater than 20% compared to untreated cells.

By way of further example, a MET agonist (for example a MET agonist antibody) may increase the number of branches per spheroid in cell spheroid preparations exposed to the antibody or antigen binding fragment.

It is preferred that the MET agonists used according to the invention promote MET signalling to a magnitude of at least 70% of the natural ligand, HGF—that is, that the agonists are “full agonists”. In certain embodiments, the MET agonists promote signalling to a magnitude of at least 80%, optionally at least 85%, at least 90%, at least 95% or at least 96%, at least 97%, at least 98%, at least 99% or at least 100% of HGF.

In certain embodiments, if MET agonism is determined using a phosphorylation assay, the MET agonist, e.g. a MET antibody, exhibits a potency for MET with an EC50 of <1 nM. In certain embodiments, the MET agonist, e.g. a MET antibody, exhibits a potency for MET agonism of an EMAX of at least 80% (as a percentage of maximal HGF-induced activation).

In certain embodiments, if MET agonism is measured in a cell scattering assay, the MET agonist, for example a MET antibody or antigen binding fragment, induces an increase in cell scattering at least equivalent to 0.1 nM homologous HGF when the antibody concentration is 0.1-1 nM.

In certain embodiments, if MET agonism is measured in an anti-apoptosis assay, the MET agonist (for example a MET antibody or fragment thereof) exhibits an EC50 no more than 1.1× that of HGF. In certain embodiments, if MET agonism is measured in an anti-apoptosis assay, the MET agonist (for example a MET antibody or fragment thereof) exhibits an Emax cellular viability of greater than 90% that observed for HGF.

In certain embodiments, if MET agonism is measured in a branching morphogenesis assay, cells treated with the MET agonist (e.g. a MET antibody or antigen binding fragment) exhibit greater than 90% of the number of branches per spheroid induced by the same (non-zero) concentration of HGF.

HGF-MET agonists particularly preferred in all aspects of the invention are anti-MET agonist antibodies, also referred to herein as “MET agonist antibodies”, “agonist antibodies” and grammatical variations thereof. In other words, MET agonist antibodies (or antigen binding fragments thereof) for use according to the invention bind MET and promote cellular signalling via MET.

As demonstrated in the Examples, MET agonist antibodies 71D6 and 71G2 effectively promote pancreatic islet cell growth, especially islet beta cells. 71D6 and 71G2 bind an epitope on the SEMA domain of MET, in particular an epitope on blade 4-5 of the SEMA β-propeller. MET agonist antibodies binding an epitope on the SEMA domain of MET, in particular blade 4-5 of the SEMA β-propeller have therefore been demonstrated to promote pancreatic islet cell growth, especially beta cell growth.

Thus, in certain embodiments, the methods described herein comprise administering a MET agonist antibody or antigen binding fragment thereof, wherein the antibody or antigen binding fragment binds an epitope in the SEMA domain of MET. In certain preferred embodiments, the antibodies or fragments thereof binds an epitope located on a blade of the SEMA β-propeller. In certain embodiments, the epitope is located on blade 4 or 5 of SEMA β-propeller. In certain preferred embodiments, the antibody or antigen binding fragment binds an epitope located between amino acids 314-372 of MET.

As shown in the Examples, MET agonist antibodies binding the SEMA domain of MET, including 71D6, have been shown to bind to an epitope on MET that includes residue Ile367 and residue Asp371. Mutation at either of these residues impairs binding of the antibodies to MET, with mutation of both residues completely abrogating binding.

Therefore, in certain preferred embodiments the methods described herein comprise administering a MET agonist antibody or antigen binding fragment thereof, wherein the antibody or antigen binding fragment recognises an epitope comprising the amino acid residue Ile367. In certain preferred embodiments the methods described herein comprise administering a MET agonist antibody or antigen binding fragment thereof, wherein the antibody or antigen binding fragment recognises an epitope comprising the amino acid residue Asp371.

In certain preferred embodiments, the antibody or antigen binding fragment binds an epitope comprising the amino acid residues Ile367 and Asp372 of MET.

As well as MET agonist antibodies binding the SEMA domain, also described herein are agonist antibodies binding other MET domains. For example, 71G3 binds an epitope on the PSI domain of MET. As demonstrated in the Examples, antibody 71G3 is also able to promote pancreas islet cell growth in all models tested.

Thus, in certain embodiments the methods described herein comprise administering a MET agonist antibody or antigen binding fragment thereof, wherein the antibody or antigen binding fragment binds an epitope in the PSI domain of MET. In certain preferred embodiments, the antibody or antigen binding fragment binds an epitope located between amino acids 546 and 562 of MET.

As shown in the Examples, MET agonist antibodies binding the PSI domain of MET, including 71G3, have been shown to bind to an epitope on MET that includes residue Thr555. Mutation at this residue completely abrogated binding of the PSI-binding agonist antibodies to MET.

Therefore, in certain preferred embodiments the methods described herein comprise administering a MET agonist antibody or antigen binding fragment thereof, wherein the antibody or antigen binding fragment recognises an epitope comprising the amino acid residue Thr555.

Examples of MET agonist antibodies particularly suitable for use in the methods described herein are those having a combination of CDRs corresponding to the CDRs of an anti-MET antibody described herein. Therefore, in certain embodiments, the antibody or antigen binding fragment comprises a combination of VH and VL CDR sequences corresponding to a combination of VH CDRs from a MET agonist antibody described in Table 3 and the corresponding combination of VL CDRs for the same antibody in Table 4.

In certain such embodiments, the antibody or antigen binding fragment comprises a combination of CDRs corresponding to a combination of VH CDRs from a MET agonist antibody described in Table 3 and the corresponding combination of VL CDRs for the same antibody in Table 4, and further having VH and VL domains with at least 90%, optionally at least 95%, optionally at least 99%, preferably 100% sequence identity with the corresponding VH and VL sequences of the antibody described in Table 6. By way of clarification, in such embodiments the permitted variation in percentage identity of the VH and VL domain sequences is not in the CDR regions.

As demonstrated in the Examples, 71D6, 71G2, and 71G3 are MET agonist antibodies that are “full agonists” of MET. That is, on binding of these antibodies to MET, the signalling response is similar to or even exceeds the response to binding of the native HGF ligand. Each of these antibodies is demonstrated herein to effectively promote pancreatic isle cell growth. Therefore in certain preferred embodiments of all aspects and methods described herein, the method comprises administering an HGF-MET agonist that is a full agonist—that is, an agonist that upon binding promotes MET signalling to an extent similar or in excess of MET signalling upon HGF binding. Examples for measuring MET agonism and examples of the effects of full agonists have already been described herein.

Examples of MET full agonists, such as anti-MET antibodies that are full agonists include 71D6, 71G2, and 71G3, as demonstrated in the Examples. Therefore in particularly preferred embodiments of all the methods described herein, the method comprises administering a MET agonist antibody or antigen binding fragment thereof that is a full agonist of MET.

MET agonist antibodies 71D6, 71G2 and 71G3 have each been shown to effectively promote pancreatic islet cell growth. Therefore, in preferred embodiments of all aspects and methods described herein, the antibody or fragment comprises a combination of CDRs having the corresponding CDR sequences of antibody 71D6 (SEQ ID Nos: 30, 32, 34, 107, 109, and 111), of antibody 71G2 (SEQ ID NOs: 44, 46, 48, 121, 123, and 125), or of antibody 71G3 (SEQ ID Nos: 9, 11, 13, 86, 88, and 90).

In preferred embodiments of all aspects, the MET agonist is a MET agonist antibody or antigen binding fragment thereof having HCDR1 of [71D6] SEQ ID NO: 30, HCDR2 of SEQ ID NO: 32, HCDR3 of SEQ ID NO: 34, LCDR1 of SEQ ID NO: 107, LCDR2 of SEQ ID NO: 109, and LCDR3 of SEQ ID NO: 111.

In preferred such embodiments, the antibody or antigen binding fragment comprises: a VH domain comprising SEQ ID NO: 163 or a sequence at least 90% identical thereto, optionally at least 95%, at least 98% or at least 99% identical thereto; and a VL domain comprising SEQ ID NO: 164 or a sequence at least 95% thereto optionally at least 98% or at least 99% identical thereto. By way of clarification, in such embodiments the permitted variation in percentage identity of the VH and VL domain sequences is not in the CDR regions.

MET agonist antibodies for use as described herein can take various different embodiments in which both a VH domain and a VL domain are present. The term “antibody” herein is used in the broadest sense and encompasses, but is not limited to, monoclonal antibodies (including full length monoclonal antibodies), polyclonal antibodies, multispecific antibodies (e.g., bispecific antibodies), so long as they exhibit the appropriate immunological specificity for a human MET protein and for a mouse MET protein. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” comprise a portion of a full length antibody, generally the antigen binding or variable domain thereof. Examples of antibody fragments include Fab, Fab′, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-1136, 2005, the contents of which are incorporated herein by reference).

In preferred embodiments of all aspects provided herein, the MET agonist antibody or antigen-binding fragment thereof is bivalent.

In non-limiting embodiments, the MET antibodies provided herein may comprise CH1 domains and/or CL domains, the amino acid sequence of which is fully or substantially human. Therefore, one or more or any combination of the CH1 domain, hinge region, CH2 domain, CH3 domain and CL domain (and CH4 domain if present) may be fully or substantially human with respect to its amino acid sequence. Such antibodies may be of any human isotype, for example IgG1 or IgG4.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. Such antibodies may be of any human isotype, with human IgG4 and IgG1 being particularly preferred.

MET agonist antibodies may also comprise constant domains of “human” sequence which have been altered, by one or more amino acid additions, deletions or substitutions with respect to the human sequence, excepting those embodiments where the presence of a “fully human” hinge region is expressly required. The presence of a “fully human” hinge region in the MET antibodies of the invention may be beneficial both to minimise immunogenicity and to optimise stability of the antibody.

The MET agonist antibodies may be of any isotype, for example IgA, IgD, IgE, IgG, or IgM. In preferred embodiments, the antibodies are of the IgG type, for example IgG1, IgG2a and b, IgG3 or IgG4. IgG1 and IgG4 are particularly preferred. Within each of these sub-classes it is permitted to make one or more amino acid substitutions, insertions or deletions within the Fc portion, or to make other structural modifications, for example to enhance or reduce Fc-dependent functionalities.

In non-limiting embodiments, it is contemplated that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites). Depending on the intended use of the MET antibody, it may be desirable to modify the antibody of the invention with respect to its binding properties to Fc receptors, for example to modulate effector function.

In certain embodiments, the MET antibodies may comprise an Fc region of a given antibody isotype, for example human IgG1, which is modified in order to reduce or substantially eliminate one or more antibody effector functions naturally associated with that antibody isotype. In non-limiting embodiments, the MET antibody may be substantially devoid of any antibody effector functions. In this context, “antibody effector functions” include one or more or all of antibody-dependent cellular cytotoxicity (ADCC), complement-dependent cytotoxicity (CDC) and antibody-dependent cellular phagocytosis (ADCP).

The amino acid sequence of the Fc portion of the MET antibody may contain one or more mutations, such as amino acid substitutions, deletions or insertions, which have the effect of reducing one or more antibody effector functions (in comparison to a wild type counterpart antibody not having said mutation). Several such mutations are known in the art of antibody engineering. Non-limiting examples, suitable for inclusion in the MET antibodies described herein, include the following mutations in the Fc domain of human IgG4 or human IgG1: N297A, N297Q, LALA (L234A, L235A), AAA (L234A, L235A, G237A) or D265A (amino acid residues numbering according to the EU numbering system in human IgG1).

In certain embodiments of all aspects of the invention, therefore, the anti-MET agonist antibody is an agonist antibody of both human MET and mouse MET.

Pharmaceutical Compositions

Also provided in accordance with the invention are pharmaceutical compositions for use in the methods described herein. Therefore in a further aspect of the invention is provided a pharmaceutical composition comprising an HGF-MET agonist, for example an anti-MET agonist antibody, and a pharmaceutically acceptable excipient or carrier for use in a method according to the invention. Suitable pharmaceutically acceptable carriers and excipients would be familiar to the skilled person. Examples of pharmaceutically acceptable carriers and excipients suitable for inclusion in pharmaceutical compositions of the invention include sodium citrate, glycine, polysorbate (e.g. polysorbate 80) and saline solution.

In certain embodiments, the MET agonist, for example anti-MET agonist antibody, is administered to the subject parenterally, preferably intravenously (i.v.). In certain embodiments the MET agonist, for example anti-MET agonist antibody, is administered as a continuous i.v. infusion until the desired dose is achieved.

In certain embodiments, the MET agonist, for example anti-MET agonist antibody, is administered to the subject parenterally, preferably intraperitoneally (i.p.).

### EXAMPLES

The invention will be further understood with reference to the following non-limiting experimental examples.

### Example 1: Generation of Anti-MET Agonist Antibodies—Immunization of Llamas

Immunizations of llamas and harvesting of peripheral blood lymphocytes (PBLs) as well as the subsequent extraction of RNA and amplification of antibody fragments were performed as described (De Haard et al., J. Bact. 187:4531-4541, 2005). Two adult llamas (Lama glama) were immunized by intramuscular injection of a chimeric protein consisting of the extracellular domain (ECD) of human MET fused to the Fc portion of human IgG1 (MET-Fc; R&D Systems). Each llama received one injection per week for six weeks, for a total of six injections. Each injection consisted in 0.2 mg protein in Freund's Incomplete Adjuvant in the neck divided over two spots.

Blood samples of 10 ml were collected pre- and post-immunization to investigate the immune response. Approximately one week after the last immunization, 400 ml of blood was collected and PBLs were obtained using the Ficoll-Paque method. Total RNA was extracted by the phenol-guanidine thiocyanate method (Chomczynski et al., Anal. Biochem. 162:156-159, 1987) and used as template for random cDNA synthesis using the SuperScript™ III First-Strand Synthesis System kit (Life Technologies). Amplification of the cDNAs encoding the VH-CH1 regions of llama IgG1 and VL-CL domains (κ and λ) and subcloning into the phagemid vector pCB3 was performed as described (de Haard et al., J Biol Chem. 274:18218-18230, 1999). The E. coli strain TG1 (Netherland Culture Collection of Bacteria) was transformed using recombinant phagemids to generate 4 different Fab-expressing phage libraries (one λ and one κ library per immunized llama). Diversity was in the range of 10⁸-10⁹.

The immune response to the antigen was investigated by ELISA. To this end, we obtained the ECDs of human MET (UniProtKB #P08581; aa 1-932) and of mouse MET (UniProtKB #P16056.1, aa 1-931) by standard protein engineering techniques. Human or mouse MET ECD recombinant protein was immobilized in solid phase (100 ng/well in a 96-well plate) and exposed to serial dilutions of sera from llamas before (day 0) or after (day 45) immunization. Binding was revealed using a mouse anti-llama IgG1 (Daley et al., Clin. Vaccine Immunol. 12, 2005) and a HRP-conjugated donkey anti-mouse antibody (Jackson Laboratories). Both llamas displayed an immune response against human MET ECD. Consistent with the notion that the extracellular portion of human MET displays 87% homology with its mouse orthologue, a fairly good extent of cross-reactivity was also observed with mouse MET ECD.

### Example 2: Selections and Screenings of Fabs Binding to Both Human and Mouse MET

Fab-expressing phages from the libraries described above were produced according to standard phage display protocols. For selection, phages were first adsorbed to immobilized recombinant human MET ECD, washed, and then eluted using trypsin. After two cycles of selection with human MET ECD, two other cycles were performed in the same fashion using mouse MET ECD. In parallel, we also selected phages alternating a human MET ECD cycle with a mouse MET ECD cycle, for a total of four cycles. Phages selected by the two approaches were pooled together and then used to infect TG1 E. coli. Individual colonies were isolated and secretion of Fabs was induced using IPTG (Fermentas). The Fab-containing periplasmic fraction of bacteria was collected and tested for its ability to bind human and mouse MET ECD by Surface Plasmon Resonance (SPR). Human or mouse MET ECD was immobilized on a CM-5 chip using amine coupling in sodium acetate buffer (GE Healthcare). The Fab-containing periplasmic extracts were loaded into a BIACORE 3000 apparatus (GE Healthcare) with a flow rate of 30 μl/min. The Fab off-rates (k_{off}) were measured over a two minute period. Binding of Fabs to human and mouse MET was further characterized by ELISA using MET ECD in solid phase and periplasmic crude extract in solution. Because Fabs are engineered with a MYC flag, binding was revealed using HRP-conjugated anti-MYC antibodies (ImTec Diagnostics).

Fabs that bound to both human and mouse MET in both SPR and ELISA were selected and their corresponding phages were sequenced (LGC Genomics). Cross-reactive Fab sequences were divided into families based on VH CDR3 sequence length and content. VH families were given an internal number not based on IMTG (International Immunogenetics Information System) nomenclature. Altogether, we could identify 11 different human/mouse cross-reactive Fabs belonging to 8 VH families. The CDR and FR sequences of heavy chain variable regions are shown in Table 3. The CDR and FR sequences of light chain variable regions are shown in Table 4. The full amino acid sequences of heavy chain and light chain variable regions are shown in Table 5. The full DNA sequences of heavy chain and light chain variable regions are shown in Table 6.

TABLE 3
Framework regions and CDR sequences for VH domains of Fabs binding to both human and mouse MET.

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 76H10 | QLQLVES | 1 | TYYMT | 2 | WVRQAP | 3 | DINSGG | 4 | RFTISRDNAKN | 5 | VRIWPVG | 6 | WGQGT | 7 |
| | GGGLVQP | | | | GKGLEW | | GTYYAD | | TLYLQMNSLKP | | YDY | | QVTVS | |
| | GGSLRVS | | | | VS | | SVKG | | EDTALYYCVR | | | | S | |
| | CTASGFT | | | | | | | | | | | | | |
| | FN | | | | | | | | | | | | | |
| 71G3 | QVQLVES | 8 | TYYMS | 9 | WVRQAP | 10 | DIRTDG | 11 | RFTMSRDNAKN | 12 | TRIFPSG | 13 | WGQGT | 14 |
| | GGGLVQP | | | | GKGLEW | | GTYYAD | | TLYLQMNSLKP | | YDY | | QVTVS | |
| | GGSLRVS | | | | VS | | SVKG | | EDTALYYCAR | | | | S | |
| | CAASGFT | | | | | | | | | | | | | |
| | FS | | | | | | | | | | | | | |
| 71C3 | QLQLVES | 15 | SHAMS | 16 | WVRQAP | 17 | AINSGG | 18 | RFTISRDNAKN | 19 | ELRFDLA | 20 | WGQGT | 21 |
| | GGGLVQP | | | | GKGLEW | | GSTSYA | | TLYLQMNSLKP | | RYTDYEA | | QVTVS | |
| | GGSLRLS | | | | VS | | DSVKG | | EDTAVYYCAK | | WDY | | S | |
| | CAASGFT | | | | | | | | | | | | | |
| | FS | | | | | | | | | | | | | |
| 71D4 | ELQLVES | 22 | GYGMS | 23 | WVRQAP | 24 | DINSGG | 25 | RFTISRDNAKN | 26 | DMRLYLA | 27 | WGQGT | 28 |
| | GGGLVQP | | | | GKGLEW | | GSTSYA | | TLYLQMNSLKP | | RYNDYEA | | QVTVS | |
| | GGSLRLS | | | | VS | | DSVKG | | EDTAVYYCAK | | WDY | | S | |
| | CAASGFT | | | | | | | | | | | | | |
| | FS | | | | | | | | | | | | | |
| 71D6 | ELQLVES | 29 | SYGMS | 30 | WVRQAP | 31 | AINSYG | 32 | RFTISRDNAKN | 33 | EVRADLS | 34 | WGQGT | 35 |
| | GGGLVQP | | | | GKGLEW | | GSTSYA | | TLYLQMNSLKP | | RYNDYES | | QVTVS | |
| | GGSLRLS | | | | VS | | DSVKG | | EDTAVYYCAK | | YDY | | S | |
| | CAASGFT | | | | | | | | | | | | | |
| | FS | | | | | | | | | | | | | |
| 71A3 | EVQLVES | 36 | DYDIT | 37 | WVRQAP | 38 | TITSRS | 39 | RFTISGDNAKN | 40 | VYATTWD | 41 | WGKGT | 42 |
| | GGGLVQP | | | | GKGLEW | | GSTSYV | | TLYLQMNSLKP | | VGPLGYG | | LVTVS | |
| | GGSLRLS | | | | VS | | DSVKG | | EDTAVYYCAK | | MDY | | S | |
| | CAASGFS | | | | | | | | | | | | | |
| | FK | | | | | | | | | | | | | |
| 71G2 | EVQLQES | 43 | IYDMS | 44 | WVRQAP | 45 | TINSDG | 46 | RFTISRDNAKN | 47 | VYGSTWD | 48 | WGKGT | 49 |
| | GGGLVQP | | | | GKGLEW | | SSTSYV | | TLYLQMNSLKP | | VGPMGYG | | LVTVS | |
| | GGSLRLS | | | | VS | | DSVKG | | EDTAVYYCAK | | MDY | | S | |
| | CAASGFT | | | | | | | | | | | | | |
| | FS | | | | | | | | | | | | | |
| 76G7 | QVQLVES | 50 | NYYMS | 51 | WVRQAP | 52 | DIYSDG | 53 | RFTISRDNAKN | 54 | VKIYPGG | 55 | WGQGT | 56 |
| | GGNLVQP | | | | GKGLEW | | STTWYS | | TLSLQMNSLKS | | YDA | | QVTVS | |
| | GGSLRLS | | | | VS | | DSVKG | | EDTAVYYCAR | | | | S | |
| | CAASGFT | | | | | | | | | | | | | |
| | FS | | | | | | | | | | | | | |
| 71G12 | QVQLQES | 57 | RYYMS | 58 | WVRQAP | 59 | SIDSYG | 60 | RFTISRDNAKN | 61 | AKTTWSY | 62 | WGQGT | 63 |
| | GGDLVQP | | | | GKGLEW | | YSTYYT | | TLYLQMNSLKP | | DY | | QVTVS | |
| | GGSLRVS | | | | VS | | DSVKG | | EDTALYYCAR | | | | S | |
| | CVVSGFT | | | | | | | | | | | | | |
| | FS | | | | | | | | | | | | | |
| 74C8 | EVQLVES | 64 | NYHMS | 65 | WVRQVP | 66 | DINSAG | 67 | RFTISRDNAKN | 68 | VNVWGVN | 69 | WGKGT | 70 |
| | GGGLVQP | | | | GKGFEW | | GSTYYA | | TLYLEMNSLKP | | Y | | LVSVS | |
| | GGSLRLS | | | | IS | | DSVKG | | EDIALYYCAR | | | | S | |
| | CAASGFT | | | | | | | | | | | | | |
| | FR | | | | | | | | | | | | | |
| 72F8 | ELQLVES | 71 | NYVMS | 72 | WVRQAP | 73 | DTNSGG | 74 | RFTISRDNAKN | 75 | SFFYGMN | 76 | WGKGT | 77 |
| | GGGLVQP | | | | GKGLEW | | STSYAD | | TLYLQMNSLKP | | Y | | QVTVS | |
| | GGSLRLS | | | | VS | | SVKG | | EDIALYYCAR | | | | S | |
| | CAASGFT | | | | | | | | | | | | | |
| | FS | | | | | | | | | | | | | |

TABLE 4
Framework regions and CDR sequences for VL domains of Fabs binding to both human and mouse MET.

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 76H10 | QAVVTQE | 78 | GLSSGSV | 79 | WFQQTPG | 80 | NTNNRHS | 81 | GVPSRFSGSIS | 82 | SLYTG | 83 | FGGGT | 84 |
| | PSLSVSP | | TTSNYPG | | QAPRTLI | | | | GNKAALTITGA | | SYTTV | | HLTVL | |
| | GGTVTLT | | | | Y | | | | QPEDEADYYC | | | | | |
| | C | | | | | | | | | | | | | |
| 71G3 | QAVVTQE | 85 | GLSSGSV | 86 | WFQQTPG | 87 | NITSRHS | 88 | GVPSRFSGSIS | 89 | SLYPG | 90 | FGGGT | 91 |
| | PSLSVSP | | TTSNYPG | | QAPRTLI | | | | GNKAALTIMGA | | STTV | | HLTVL | |
| | GGTVTLT | | | | Y | | | | QPEDEADYYC | | | | | |
| | C | | | | | | | | | | | | | |
| 71C3 | SYELTQP | 92 | QGGSLGS | 93 | WYQQKPG | 94 | DDDSRPS | 95 | GIPERFSGSSS | 96 | QSADS | 97 | FGGGT | 98 |
| | SALSVTL | | SYAH | | QAPVLVI | | | | GGTATLTISGA | | SGNAA | | HLTVL | |
| | GQTAKIT | | | | Y | | | | QAEDEGDYYC | | V | | | |
| | C | | | | | | | | | | | | | |
| 71D4 | SSALTQP | 99 | QGGSLGS | 100 | WYQQKPG | 101 | DDDSRPS | 102 | GIPERFSGSSS | 103 | QSADS | 104 | FGGGT | 105 |
| | SALSVTL | | SYAH | | QAPVLVI | | | | GGTATLTISGA | | SGNAA | | HLTVL | |
| | GQTAKIT | | | | Y | | | | QAEDEGDYYC | | V | | | |
| | C | | | | | | | | | | | | | |
| 71D6 | QPVLNQP | 106 | QGGSLGA | 107 | WYQQKPG | 108 | DDDSRPS | 109 | GIPERFSGSSS | 110 | QSADS | 111 | FGGGT | 112 |
| | SALSVTL | | RYAH | | QAPVLVI | | | | GGTATLTISGA | | SGSV | | HLTVL | |
| | GQTAKIT | | | | Y | | | | QAEDEGDYYC | | | | | |
| | C | | | | | | | | | | | | | |
| 71A3 | SYELTQP | 113 | QGGSLGS | 114 | WYQQKPG | 115 | DDDSRPS | 116 | GIPERFSGSSS | 117 | QSADS | 118 | FGGGT | 119 |
| | SALSVTL | | SYAH | | QAPVLVI | | | | GGTATLTISGA | | SGNAA | | HLTVL | |
| | GQTAKIT | | | | Y | | | | QAEDEGDYYC | | V | | | |
| | C | | | | | | | | | | | | | |
| 71G2 | SSALTQP | 120 | QGGSLGS | 121 | WYQQKPG | 122 | GDDSRPS | 123 | GIPERFSGSSS | 124 | QSTDS | 125 | FGGGT | 126 |
| | SALSVSL | | SYAH | | QAPVLVI | | | | GGTATLTISGA | | SGNTV | | RLTVL | |
| | GQTARIT | | | | Y | | | | QAEDEDDYYC | | | | | |
| | C | | | | | | | | | | | | | |
| 76G7 | QAGLTQP | 127 | AGNSSDV | 128 | WYQQFPG | 129 | LVNKRAS | 130 | GITDRFSGSKS | 131 | ASYTG | 132 | FGGGT | 133 |
| | PSVSGSP | | GYGNYVS | | MAPKLLI | | | | GNTASLTISGL | | SNNIV | | HLTVL | |
| | GKTVTIS | | | | Y | | | | QSEDEADYYC | | | | | |
| | C | | | | | | | | | | | | | |
| 71G12 | EIVLTQS | 134 | KSSQSVF | 135 | WYQQRPG | 136 | YASTRES | 137 | GIPDRFSGSGS | 138 | QQAYS | 139 | FGQGT | 140 |
| | PSSVTAS | | IASNQKT | | QSPRLVI | | | | TTDFTLTISSV | | HPT | | KVELK | |
| | VGGKVTI | | YLN | | S | | | | QPEDAAVYYC | | | | | |
| | NC | | | | | | | | | | | | | |
| 74C8 | QTVVTQE | 141 | GLSSGSV | 142 | WFQQTPG | 143 | NITSRHS | 144 | GVPSRFSGSIS | 145 | SLYPG | 146 | FGGGT | 147 |
| | PSLSVSP | | TTSNYPG | | QAPRTLI | | | | GNKAALTITGA | | SYTNV | | HLTVL | |
| | GGTVTLT | | | | Y | | | | QPEDEADYYC | | | | | |
| | C | | | | | | | | | | | | | |
| 72F8 | QSALTQP | 148 | TLSSGNN | 149 | WYQQKAG | 150 | YYTDSRK | 151 | GVPSRFSGSKD | 152 | SAYKS | 153 | FGGGT | 154 |
| | PSLSASP | | IGSYDIS | | SPPRYLL | | HQDS | | ASANAGLLLIS | | GSYRW | | HVTVL | |
| | GSSVRLT | | | | N | | | | GLQPEDEADYY | | V | | | |
| | C | | | | | | | | C | | | | | |

TABLE 5
Variable domain amino acid sequences of Fabs binding to both human and mouse MET.

| | | SEQ ID | | SEQ ID |
|---|---|---|---|---|
| CLONE | VH | NO. | VL | NO. |
| 76H10 | QLQLVESGGGLVQPGGSLRVSCTASGFTFNTYYMT | 155 | QAVVTQEPSLSVSPGGTVTLTCGLSSGSVTTSNYP | 156 |
| | WVRQAPGKGLEWVSDINSGGGTYYADSVKGRFTIS | | GWFQQTPGQAPRTLIYNTNNRHSGVPSRFSGSISG | |
| | RDNAKNTLYLQMNSLKPEDTALYYCVRVRIWPVGY | | NKAALTITGAQPEDEADYYCSLYTGSYTTVFGGGT | |
| | DYWGQGTQVTVSS | | HLTVL | |
| 71G3 | QVQLVESGGGLVQPGGSLRVSCAASGFTFSTYYMS | 157 | QAVVTQEPSLSVSPGGTVTLTCGLSSGSVTTSNYP | 158 |
| | WVRQAPGKGLEWVSDIRTDGGTYYADSVKGRFTMS | | GWFQQTPGQAPRTLIYNTNSRHSGVPSRFSGSISG | |
| | RDNAKNTLYLQMNSLKPEDTALYYCARTRIFPSGY | | NKAALTIMGAQPEDEADYYCSLYPGSTTVFGGGTH | |
| | DYWGQGTQVTVSS | | LTVL | |
| 71C3 | QLQLVESGGGLVQPGGSLRLSCAASGFTFSSHAMS | 159 | SYELTQPSALSVTLGQTAKITCQGGSLGSSYAHWY | 160 |
| | WVRQAPGKGLEWVSAINSGGGSTSYADSVKGRFTI | | QQKPGQAPVLVIYDDDSRPSGIPERFSGSSSGGTA | |
| | SRDNAKNTLYLQMNSLKPEDTAVYYCAKELRFDLA | | TLTISGAQAEDEGDYYCQSADSSGNAAVFGGGTHL | |
| | RYTDYEAWDYWGQGTQVTVSS | | TVL | |
| 71D4 | ELQLVESGGGLVQPGGSLRLSCAASGFTFSGYGMS | 161 | SSALTQPSALSVTLGQTAKITCQGGSLGSSYAHWY | 162 |
| | WVRQAPGKGLEWVSDINSGGGSTSYADSVKGRFTI | | QQKPGQAPVLVIYDDDSRPSGIPERFSGSSSGGTA | |
| | SRDNAKNTLYLQMNSLKPEDTAVYYCAKDMRLYLA | | TLTISGAQAEDEGDYYCQSADSSGNAAVFGGGTHL | |
| | RYNDYEAWDYWGQGTQVTVSS | | TVL | |
| 71D6 | ELQLVESGGGLVQPGGSLRLSCAASGFTFSSYGMS | 163 | QPVLNQPSALSVTLGQTAKITCQGGSLGARYAHWY | 164 |
| | WVRQAPGKGLEWVSAINSYGGSTSYADSVKGRFTI | | QQKPGQAPVLVIYDDDSRPSGIPERFSGSSSGGTA | |
| | SRDNAKNTLYLQMNSLKPEDTAVYYCAKEVRADLS | | TLTISGAQAEDEGDYYCQSADSSGSVFGGGTHLTV | |
| | RYNDYESYDYWGQGTQVTVSS | | L | |
| 71A3 | EVQLVESGGGLVQPGGSLRLSCAASGFSFKDYDIT | 165 | SYELTQPSALSVTLGQTAKITCQGGSLGSSYAHWY | 166 |
| | WVRQAPGKGLEWVSTITSRSGSTSYVDSVKGRFTI | | QQKPGQAPVLVIYDDDSRPSGIPERFSGSSSGGTA | |
| | SGDNAKNTLYLQMNSLKPEDTAVYYCAKVYATTWD | | TLTISGAQAEDEGDYYCQSADSSGNAAVFGGGTHL | |
| | VGPLGYGMDYWGKGTLVTVSS | | TVL | |
| 71G2 | EVQLQESGGGLVQPGGSLRLSCAASGFTFSIYDMS | 167 | SSALTQPSALSVSLGQTARITCQGGSLGSSYAHWY | 168 |
| | WVRQAPGKGLEWVSTINSDGSSTSYVDSVKGRFTI | | QQKPGQAPVLVIYGDDSRPSGIPERFSGSSSGGTA | |
| | SRDNAKNTLYLQMNSLKPEDTAVYYCAKVYGSTWD | | TLTISGAQAEDEDDYYCQSTDSSGNTVFGGGTRLT | |
| | VGPMGYGMDYWGKGTLVTVSS | | VL | |
| 76G7 | QVQLVESGGNLVQPGGSLRLSCAASGFTFSNYYMS | 169 | QAGLTQPPSVSGSPGKTVTISCAGNSSDVGYGNYV | 170 |
| | WVRQAPGKGLEWVSDIYSDGSTTWYSDSVKGRFTI | | SWYQQFPGMAPKLLIYLVNKRASGITDRFSGSKSG | |
| | SRDNAKNTLSLQMNSLKSEDTAVYYCARVKIYPGG | | NTASLTISGLQSEDEADYYCASYTGSNNIVFGGGT | |
| | YDAWGQGTQVTVSS | | HLTVL | |
| 71G12 | QVQLQESGGDLVQPGGSLRVSCVVSGFTFSRYYMS | 171 | EIVLIQSPSSVTASVGGKVTINCKSSQSVFIASNQ | 172 |
| | WVRQAPGKGLEWVSSIDSYGYSTYYTDSVKGRFTI | | KTYLNWYQQRPGQSPRLVISYASTRESGIPDRFSG | |
| | SRDNAKNTLYLQMNSLKPEDTALYYCARAKTTWSY | | SGSTTDFTLTISSVQPEDAAVYYCQQAYSHPTFGQ | |
| | DYWGQGTQVTVSS | | GTKVELK | |
| 74C8 | EVQLVESGGGLVQPGGSLRLSCAASGFTFRNYHMS | 173 | QTVVTQEPSLSVSPGGTVTLTCGLSSGSVTTSNYP | 174 |
| | WVRQVPGKGFEWISDINSAGGSTYYADSVKGRFTI | | GWFQQTPGQAPRTLIYNTNSRHSGVPSRFSGSISG | |
| | SRDNAKNTLYLEMNSLKPEDTALYYCARVNVWGVN | | NKAALTITGAQPEDEADYYCSLYPGSYTNVFGGGT | |
| | YWGKGTLVSVSS | | HLTVL | |
| 72F8 | ELQLVESGGGLVQPGGSLRLSCAASGFTFSNYVMS | 175 | QSALTQPPSLSASPGSSVRLTCTLSSGNNIGSYDI | 176 |
| | WVRQAPGKGLEWVSDINSGGSTSYADSVKGRFTIS | | SWYQQKAGSPPRYLLNYYTDSRKHQDSGVPSRFSG | |
| | RDNAKNTLYLQMNSLKPEDTALYYCARSFFYGMNY | | SKDASANAGLLLISGLQPEDEADYYCSAYKSGSYR | |
| | WGKGTQVTVSS | | WVFGGGTHVTVL | |

TABLE 6
Variable domain nucleotide sequences of Fabs binding to both human and mouse MET.

| | | SEQ ID | | SEQ ID |
|---|---|---|---|---|
| Clone | VH | NO. | VL | NO. |
| 76H10 | CAGTTGCAGCTGGTGGAGTCTGGGGGAGGCTTGGT | 177 | CAGGCTGTGGTGACCCAGGAGCCGTCCCTGTCAGT | 178 |
| | GCAGCCTGGGGGGTCTCTGAGAGTTTCCTGTACAG | | GTCTCCAGGAGGGACGGTCACACTCACCTGCGGCC | |
| | CCTCTGGATTCACCTTCAATACCTACTACATGACC | | TCAGCTCTGGGTCTGTCACTACCAGTAACTACCCT | |
| | TGGGTCCGCCAGGCTCCAGGGAAGGGGCTCGAGTG | | GGTTGGTTCCAGCAGACACCGGGCCAGGCTCCACG | |
| | GGTCTCAGATATTAATAGTGGTGGTGGTACATACT | | CACTCTTATCTACAACACAAACAACCGCCACTCTG | |
| | ATGCAGACTCCGTGAAGGGCCGATTCACCATCTCC | | GGGTCCCCAGTCGCTTCTCCGGATCCATCTCTGGG | |
| | AGAGACAACGCCAAGAACACGCTATATCTGCAAAT | | AACAAAGCCGCCCTCACCATCACGGGGGCCCAGCC | |
| | GAACAGCCTGAAACCTGAGGACACGGCCCTGTATT | | CGAGGACGAGGCCGACTATTACTGTTCTCTATATA | |
| | ACTGTGTAAGAGTTCGTATTTGGCCAGTGGGATAT | | CTGGCAGTTACACTACTGTGTTCGGCGGAGGGACC | |
| | GACTACTGGGGCCAGGGGACCCAGGTCACCGTTTC | | CATCTGACCGTCCTG | |
| | CTCA | | | |
| 71G3 | CAGGTGCAGCTCGTGGAGTCTGGGGGAGGCTTGGT | 179 | CAGGCTGTGGTGACCCAGGAGCCGTCCCTGTCAGT | 180 |
| | GCAGCCTGGGGGGTCTCTGAGAGTCTCCTGTGCAG | | GTCTCCAGGAGGGACGGTCACACTCACCTGCGGCC | |
| | CCTCTGGATTCACCTTCAGTACCTACTACATGAGC | | TCAGCTCTGGGTCTGTCACTACCAGTAACTACCCT | |
| | TGGGTCCGCCAGGCTCCAGGGAAGGGGCTCGAGTG | | GGTTGGTTCCAGCAGACACCAGGCCAGGCTCCGCG | |
| | GGTCTCAGATATTCGTACTGATGGTGGCACATACT | | CACTCTTATCTACAACACAAACAGCCGCCACTCTG | |
| | ATGCAGACTCCGTGAAGGGCCGATTCACCATGTCC | | GGGTCCCCAGTCGCTTCTCCGGATCCATCTCTGGG | |
| | AGAGACAACGCCAAGAACACGCTGTATCTACAAAT | | AACAAAGCCGCCCTCACCATCATGGGGGCCCAGCC | |
| | GAACAGCCTGAAACCTGAGGACACGGCCCTGTATT | | CGAGGACGAGGCCGACTATTACTGTTCTCTGTACC | |
| | ACTGTGCAAGAACTCGAATTTTCCCCTCGGGGTAT | | CTGGTAGTACCACTGTGTTCGGCGGAGGGACCCAT | |
| | GACTACTGGGGCCAGGGGACCCAGGTCACCGTCTC | | CTGACCGTCCTG | |
| | CTCA | | | |
| 71C3 | CAGTTGCAGCTGGTGGAGTCTGGGGGAGGCTTGGT | 181 | TCCTATGAGCTGACTCAGCCCTCCGCGCTGTCCGT | 182 |
| | GCAGCCTGGGGGGTCTCTGAGACTCTCCTGTGCAG | | AACCTTGGGACAGACGGCCAAGATCACCTGCCAAG | |
| | CCTCTGGATTCACCTTCAGTAGCCATGCCATGAGC | | GTGGCAGCTTAGGTAGCAGTTATGCTCACTGGTAC | |
| | TGGGTCCGCCAGGCTCCAGGAAAGGGGCTCGAGTG | | CAGCAGAAGCCAGGCCAGGCCCCTGTGCTGGTCAT | |
| | GGTCTCAGCTATTAATAGTGGTGGTGGTAGCACAA | | CTATGATGATGACAGCAGGCCCTCAGGGATCCCTG | |
| | GCTATGCAGACTCCGTGAAGGGCCGATTCACCATC | | AGCGGTTCTCTGGCTCCAGCTCTGGGGGCACAGCC | |
| | TCCAGAGACAACGCCAAGAACACGCTGTACCTGCA | | ACCCTGACCATCAGCGGGGCCCAGGCCGAGGACGA | |
| | AATGAACAGCCTGAAACCTGAGGACACGGCCGTGT | | GGGTGACTATTACTGTCAGTCAGCAGACAGCAGTG | |
| | ATTACTGTGCAAAAGAGCTGAGATTCGACCTAGCA | | GTAATGCTGCTGTGTTCGGCGGAGGGACCCATCTG | |
| | AGGTATACCGACTATGAGGCCTGGGACTACTGGGG | | ACCGTCCTG | |
| | CCAGGGGACCCAGGTCACCGTCTCCTCA | | | |
| 71D4 | GAGTTGCAGCTGGTGGAGTCTGGGGGAGGCTTGGT | 183 | TCCTCTGCACTGACTCAGCCCTCCGCGCTGTCCGT | 184 |
| | GCAGCCTGGGGGGTCTCTGAGACTCTCCTGTGCAG | | AACCTTGGGACAGACGGCCAAGATCACCTGCCAAG | |
| | CCTCTGGATTCACCTTCAGTGGCTATGGCATGAGC | | GTGGCAGCTTAGGTAGCAGTTATGCTCACTGGTAC | |
| | TGGGTCCGCCAGGCTCCAGGAAAGGGGCTCGAGTG | | CAGCAGAAGCCAGGCCAGGCCCCTGTGCTGGTCAT | |
| | GGTCTCAGATATTAATAGTGGTGGTGGTAGCACAA | | CTATGATGATGACAGCAGGCCCTCAGGGATCCCTG | |
| | GCTATGCAGACTCCGTGAAGGGCCGATTCACCATC | | AGCGGTTCTCTGGCTCCAGCTCTGGGGGCACAGCC | |
| | TCCAGAGACAACGCCAAGAACACGCTGTATCTGCA | | ACCCTGACCATCAGCGGGGCCCAGGCCGAGGACGA | |
| | AATGAACAGCCTGAAACCTGAGGACACGGCCGTGT | | GGGTGACTATTACTGTCAGTCAGCAGACAGCAGTG | |
| | ATTACTGTGCAAAAGATATGAGATTATACCTAGCA | | GTAATGCTGCTGTGTTCGGCGGAGGGACCCATCTG | |
| | AGGTATAACGACTATGAGGCCTGGGACTACTGGGG | | ACCGTCCTG | |
| | CCAGGGGACCCAGGTCACCGTCTCCTCA | | | |
| 71D6 | GAGTTGCAGCTGGTGGAGTCTGGGGGAGGCTTGGT | 185 | CAGCCGGTGCTGAATCAGCCCTCCGCGCTGTCCGT | 186 |
| | GCAGCCTGGGGGGTCTCTGAGACTCTCCTGTGCAG | | AACCTTGGGACAGACGGCCAAGATCACCTGCCAAG | |
| | CCTCTGGATTCACCTTCAGTAGCTATGGCATGAGC | | GTGGCAGCTTAGGTGCGCGTTATGCTCACTGGTAC | |
| | TGGGTCCGCCAGGCTCCAGGAAAGGGGCTCGAGTG | | CAGCAGAAGCCAGGCCAGGCCCCTGTGCTGGTCAT | |
| | GGTCTCAGCTATTAATAGTTATGGTGGTAGCACAA | | CTATGATGATGACAGCAGGCCCTCAGGGATCCCTG | |
| | GCTATGCAGACTCCGTGAAGGGCCGATTCACCATC | | AGCGGTTCTCTGGCTCCAGCTCTGGGGGCACAGCC | |
| | TCCAGAGACAACGCCAAGAACACGCTGTATCTGCA | | ACCCTGACCATCAGCGGGGCCCAGGCCGAGGACGA | |
| | AATGAACAGCCTGAAACCTGAGGACACGGCCGTGT | | GGGTGACTATTACTGTCAGTCAGCAGACAGCAGTG | |
| | ATTACTGTGCAAAAGAAGTGCGGGCCGACCTAAGC | | GTTCTGTGTTCGGCGGAGGGACCCATCTGACCGTC | |
| | CGCTATAACGACTATGAGTCGTATGACTACTGGGG | | CTG | |
| | CCAGGGGACCCAGGTCACCGTCTCCTCA | | | |
| 71A3 | GAGGTGCAGCTCGTGGAGTCTGGGGGAGGCTTGGT | 187 | TCCTATGAGCTGACTCAGCCCTCCGCGCTGTCCGT | 188 |
| | GCAGCCTGGGGGGTCTCTGAGACTCTCCTGTGCAG | | AACCTTGGGACAGACGGCCAAGATCACCTGCCAAG | |
| | CCTCTGGATTCAGCTTCAAGGACTATGACATAACC | | GTGGCAGCTTAGGTAGCAGTTATGCTCACTGGTAC | |
| | TGGGTCCGCCAGGCTCCGGGAAAGGGGCTCGAGTG | | CAGCAGAAGCCAGGCCAGGCCCCTGTGCTGGTCAT | |
| | GGTCTCAACTATTACTAGTCGTAGTGGTAGCACAA | | CTATGATGATGACAGCAGGCCCTCAGGGATCCCTG | |
| | GCTATGTAGACTCCGTAAAGGGCCGATTCACCATC | | AGCGGTTCTCTGGCTCCAGCTCTGGGGGCACAGCC | |
| | TCCGGAGACAACGCCAAGAACACGCTGTATCTGCA | | ACCCTGACCATCAGCGGGGCCCAGGCCGAGGACGA | |
| | AATGAACAGCCTGAAACCTGAGGACACGGCCGTGT | | GGGTGACTATTACTGTCAGTCAGCAGACAGCAGTG | |
| | ATTACTGTGCAAAAGTTTACGCGACTACCTGGGAC | | GTAATGCTGCTGTGTTCGGCGGAGGGACCCATCTG | |
| | GTCGGCCCTCTGGGCTACGGCATGGACTACTGGGG | | ACCGTCCTG | |
| | CAAGGGGACCCTGGTCACCGTCTCCTCA | | | |
| 71G2 | GAGGTGCAGCTGCAGGAGTCGGGGGGAGGCTTGGT | 189 | TCCTCTGCACTGACTCAGCCCTCCGCGCTGTCCGT | 190 |
| | GCAGCCTGGGGGGTCTCTGAGACTCTCCTGTGCAG | | GTCCTTGGGACAGACGGCCAGGATCACCTGCCAAG | |
| | CCTCTGGATTCACCTTCAGTATATATGACATGAGC | | GTGGCAGCTTAGGTAGCAGTTATGCTCACTGGTAC | |
| | TGGGTCCGCCAGGCTCCAGGAAAGGGGCTCGAGTG | | CAGCAGAAGCCAGGCCAGGCCCCTGTGCTGGTCAT | |
| | GGTCTCAACTATTAATAGTGATGGTAGTAGCACAA | | CTATGGTGATGACAGCAGGCCCTCAGGGATCCCTG | |
| | GCTATGTAGACTCCGTGAAGGGCCGATTCACCATC | | AGCGGTTCTCTGGCTCCAGCTCTGGGGGCACAGCC | |
| | TCCAGAGACAACGCCAAGAACACGCTGTATCTGCA | | ACCCTGACCATCAGCGGGGCCCAGGCCGAGGACGA | |
| | AATGAACAGCCTGAAACCTGAGGACACGGCCGTGT | | GGATGACTATTACTGTCAGTCAACAGACAGCAGTG | |
| | ATTACTGTGCGAAAGTTTACGGTAGTACCTGGGAC | | GTAATACTGTGTTCGGCGGAGGGACCCGACTGACC | |
| | GTCGGCCCTATGGGCTACGGCATGGACTACTGGGG | | GTCCTG | |
| | CAAAGGGACCCTGGTCACTGTCTCCTCA | | | |
| 76G7 | CAGGTGCAGCTGGTGGAGTCTGGGGGAAACTTGGT | 191 | CAGGCAGGGCTGACTCAGCCTCCCTCCGTGTCTGG | 192 |
| | GCAGCCTGGGGGTTCTCTGAGACTCTCCTGTGCAG | | GTCTCCAGGAAAGACGGTCACCATCTCCTGTGCAG | |
| | CCTCTGGATTCACCTTCAGTAACTACTACATGAGC | | GAAACAGCAGTGATGTTGGGTATGGAAACTATGTC | |
| | TGGGTCCGCCAGGCTCCAGGGAAGGGGCTGGAATG | | TCCTGGTACCAGCAGTTCCCAGGAATGGCCCCCAA | |
| | GGTGTCCGATATTTATAGTGACGGTAGTACCACAT | | ACTCCTGATATATCTCGTCAATAAACGGGCCTCAG | |
| | GGTATTCAGACTCCGTCAAGGGCCGATTCACCATC | | GGATCACTGATCGCTTCTCTGGCTCCAAGTCAGGC | |
| | TCCAGAGACAACGCCAAGAACACGCTGTCTCTGCA | | AACACGGCCTCCCTGACCATCTCTGGGCTCCAGTC | |
| | AATGAACAGTCTGAAATCTGAGGACACGGCCGTCT | | TGAGGACGAGGCTGATTATTACTGTGCCTCATATA | |
| | ATTACTGTGCGCGCGTGAAGATCTATCCGGGGGGG | | CAGGTAGCAACAATATCGTGTTCGGCGGAGGGACC | |
| | TATGACGCCTGGGGCCAGGGGACCCAGGTCACCGT | | CATCTAACCGTCCTC | |
| | CTCCTCA | | | |
| 71G12 | CAGGTGCAGCTGCAGGAGTCGGGGGGAGACTTGGT | 193 | GAAATTGTGTTGACGCAGTCTCCCAGCTCCGTGAC | 194 |
| | GCAGCCTGGGGGGTCTCTGAGAGTCTCCTGTGTAG | | TGCATCTGTAGGAGGGAAGGTCACTATCAACTGTA | |
| | TCTCTGGATTCACCTTCAGTCGCTACTACATGAGC | | AGTCCAGCCAGAGCGTCTTCATAGCTTCTAATCAG | |
| | TGGGTCCGCCAGGCTCCAGGGAAGGGGCTCGAGTG | | AAAACCTACTTAAACTGGTACCAGCAGAGACCTGG | |
| | GGTCTCATCTATTGATAGTTATGGTTACAGCACAT | | ACAGTCTCCGAGGTTGGTCATCAGCTATGCGTCCA | |
| | ACTATACAGACTCCGTGAAGGGCCGATTCACCATC | | CCCGTGAATCGGGGATCCCTGATCGATTCAGCGGC | |
| | TCCAGAGACAACGCCAAGAACACGCTGTATCTGCA | | AGTGGGTCCACAACAGATTTCACTCTCACGATCAG | |
| | AATGAACAGCCTGAAACCTGAGGACACGGCCCTGT | | CAGTGTCCAGCCTGAAGATGCGGCCGTGTATTACT | |
| | ATTACTGTGCAAGAGCGAAAACGACTTGGAGTTAT | | GTCAGCAGGCTTATAGCCATCCAACGTTCGGCCAG | |
| | GACTACTGGGGCCAGGGGACCCAGGTCACCGTCTC | | GGGACCAAGGTGGAACTCAAA | |
| | CTCA | | | |
| 74C8 | GAGGTGCAGCTCGTGGAGTCTGGGGGAGGCTTGGT | 195 | CAGACTGTGGTGACTCAGGAGCCGTCCCTGTCAGT | 196 |
| | GCAACCTGGGGGTTCTCTGAGACTCTCCTGTGCAG | | GTCTCCAGGAGGGACGGTCACACTCACCTGCGGCC | |
| | CCTCTGGATTCACCTTCAGGAATTACCACATGAGT | | TCAGCTCTGGGTCTGTCACTACCAGTAACTACCCT | |
| | TGGGTCCGCCAGGTTCCAGGGAAGGGGTTCGAGTG | | GGTTGGTTCCAGCAGACACCAGGCCAGGCTCCACG | |
| | GATCTCAGATATTAATAGTGCAGGTGGTAGCACAT | | CACTCTTATCTACAACACAAACAGCCGCCACTCTG | |
| | ACTATGCAGACTCCGTGAAGGGCCGATTCACCATC | | GGGTCCCCAGTCGCTTCTCCGGATCCATCTCTGGG | |
| | TCCAGAGACAACGCCAAGAACACGCTGTATCTGGA | | AACAAAGCCGCCCTCACCATCACGGGGGCCCAGCC | |
| | AATGAACAGCCTGAAACCTGAGGACACGGCCCTGT | | CGAGGACGAGGCCGACTATTACTGTTCTCTGTACC | |
| | ATTACTGTGCAAGAGTCAACGTCTGGGGGGTGAAC | | CTGGTAGTTACACTAATGTGTTCGGCGGAGGGACC | |
| | TACTGGGGCAAAGGGACCCTGGTCAGCGTCTCCTC | | CATCTGACCGTCCTG | |
| | A | | | |
| 72F8 | GAGTTGCAGCTGGTGGAGTCTGGGGGAGGCTTGGT | 197 | CAGTCTGCCCTGACTCAGCCGCCCTCCCTCTCTGC | 198 |
| | GCAGCCTGgGGGGTCTCTGAGACTCTcCTGTGCAG | | ATCTCCGGGATCATCTGTCAGACTCACCTGCACCC | |
| | CCTCTGGATTCACCTTCAGCAACTATGTCATGAGC | | TGAGCAGTGGAAACAATATTGGCAGCTATGACATA | |
| | TGGGTCCGCCAGGCTCCAGGAAAGGGGCTCGAGTG | | AGTTGGTACCAGCAGAAGGCAGGGAGCCCTCCCCG | |
| | GGTCTCAGATACTAATAGTGGTGGTAGCACAAGCT | | GTACCTCCTGAACTACTACACCGACTCACGCAAGC | |
| | ATGCAGACTCCGTGAAGGGCCGATTCACCATCTCT | | ACCAGGACTCCGGGGTCCCGAGCCGCTTCTCTGGG | |
| | AGAGACAACGCCAAGAACACGCTGTATTTGCAAAT | | TCCAAAGATGCCTCGGCCAACGCAGGGCTTCTGCT | |
| | GAACAGCCTGAAACCTGAGGACACGGCATTGTATT | | CATCTCTGGGCTTCAGCCCGAGGACGAGGCTGACT | |
| | ACTGTGCGAGATCATTTTTCTACGGCATGAACTAC | | ATTACTGTTCTGCATACAAGAGTGGTTCTTACCGT | |
| | TGGGGCAAAGGGACCCAGGTCACCGTGTCCTCA | | TGGGTGTTCGGCGGAGGGACGCACGTGACCGTCCT | |
| | | | G | |

The various Fab families and their ability to bind human and mouse MET are shown in Table 7.

TABLE 7
Fabs binding to both human MET (hMET) and mouse MET (mMET).
Fabs are grouped in families based on their VH CDR3 sequence.
Binding of Fabs to human and mouse MET ECD was determined
by Surface Plasmon Resonance (SPR) and by ELISA. SPR values
represent the koff (s⁻¹). ELISA values represent the Optical
Density (OD) at 450 nm (AU, arbitrary units). Both SPR and
ELISA were performed using crude periplasmic extracts.
Fab concentration in the extract was not determined.
Values are the mean of three independent measurements.

| | | | SPR (K_{off}; s⁻¹) | | ELISA (OD₄₅₀; AU) | |
|---|---|---|---|---|---|---|
| Fab | VH | VL | hMET | mMET | hMET | mMET |
| 76H10 | VH 1 | Lambda | 5.68E−03 | 5.44E−03 | 3.704 | 3.697 |
| 71G3 | VH 2 | Lambda | 1.42E−03 | 1.41E−03 | 3.462 | 3.443 |
| 71D6 | VH 3a | Lambda | 2.94E−03 | 2.67E−03 | 3.261 | 3.072 |
| 71C3 | VH 3b | Lambda | 2.25E−03 | 2.58E−03 | 1.650 | 1.643 |
| 71D4 | VH 3c | Lambda | 2.17E−03 | 2.38E−03 | 0.311 | 0.307 |
| 71A3 | VH 4 | Lambda | 4.92E−03 | 4.74E−03 | 0.581 | 0.524 |
| 71G2 | VH 4 | Lambda | 1.21E−03 | 1.48E−03 | 0.561 | 0.543 |
| 76G7 | VH 5 | Lambda | 4.32E−03 | 4.07E−03 | 3.199 | 3.075 |
| 71G12 | VH 6 | Kappa | 2.28E−03 | 2.55E−03 | 0.450 | 0.420 |
| 74C8 | VH 9 | Lambda | 3.48E−03 | 3.70E−03 | 2.976 | 2.924 |
| 72F8 | VH 10 | Lambda | 4.96E−03 | 4.58E−03 | 3.379 | 3.085 |

### Example 3: Chimerization of Fabs into mAbs

The cDNAs encoding the VH and VL (κ or λ) domains of selected Fab fragments were engineered into two separate pUPE mammalian expression vectors (U-protein Express) containing the cDNAs encoding CH1, CH2 and CH3 of human IgG1 or the human CL (κ or λ), respectively.

Production (by transient transfection of mammalian cells) and purification (by protein A affinity chromatography) of the resulting chimeric llama-human IgG1 molecules was outsourced to U-protein Express. Binding of chimeric mAbs to MET was determined by ELISA using hMET or mMET ECD in solid phase and increasing concentrations of antibodies (0-20 nM) in solution. Binding was revealed using HRP-conjugated anti-human Fc antibodies (Jackson Immuno Research Laboratories). This analysis revealed that all chimeric llama-human antibodies bound to human and mouse MET with picomolar affinity, displaying an EC₅₀ comprised between 0.06 nM and 0.3 nM. Binding capacity (E_{MAX}) varied from antibody to antibody, possibly due to partial epitope exposure in the immobilized antigen, but was similar in the human and mouse setting. EC₅₀ and E_{MAX} values are shown in Table 9.

TABLE 9
Binding of chimeric mAbs to human and mouse MET as
determined by ELISA using immobilized MET ECD in solid
phase and increasing concentrations (0-20 nM) of antibodies in solution.

| | hMET | | mMET | |
|---|---|---|---|---|
| mAb | EC₅₀ | E_{MAX} | EC₅₀ | E_{MAX} |
| 76H10 | 0.090 | 2.669 | 0.062 | 2.662 |
| 71G3 | 0.067 | 2.835 | 0.057 | 2.977 |
| 71D6 | 0.026 | 2.079 | 0.049 | 2.009 |
| 71C3 | 0.203 | 2.460 | 0.293 | 2.238 |
| 71D4 | 0.207 | 1.428 | 0.274 | 1.170 |
| 71A3 | 0.229 | 2.401 | 0.176 | 2.730 |
| 71G2 | 0.112 | 3.094 | 0.101 | 3.168 |
| 76G7 | 0.128 | 2.622 | 0.103 | 2.776 |
| 71G12 | 0.106 | 3.076 | 0.127 | 2.973 |
| 74C8 | 0.090 | 0.994 | 0.116 | 0.896 |
| 72F8 | 0.064 | 2.779 | 0.048 | 2.903 |

EC₅₀ values are expressed as nMol/L.
E_{MAX} values are expressed as Optical Density (OD) at 450 nm (AU, arbitrary units).

We also analysed whether chimeric anti-MET antibodies bound to native human and mouse MET in living cells. To this end, increasing concentrations of antibodies (0-100 nM) were incubated with A549 human lung carcinoma cells (American Type Culture Collection) or MLP29 mouse liver precursor cells (a gift of Prof. Enzo Medico, University of Torino, Strada Provinciale 142 km 3.95, Candiolo, Torino, Italy; Medico et al., Mol Biol Cell 7, 495-504, 1996), which both express physiological levels of MET. Antibody binding to cells was analysed by flow cytometry using phycoerythrin-conjugated anti-human IgG1 antibodies (eBioscience) and a CyAn ADP analyser (Beckman Coulter). As a positive control for human MET binding, we used a commercial mouse anti-human MET antibody (R&D Systems) and phycoerythrin-conjugated anti-mouse IgG1 antibodies (eBioscience). As a positive control for mouse MET binding we used a commercial goat anti-mouse MET antibody (R&D Systems) and phycoerythrin-conjugated anti-goat IgG1 antibodies (eBioscience). All antibodies displayed dose-dependent binding to both human and mouse cells with an EC₅₀ varying between 0.2 nM and 2.5 nM. Consistent with the data obtained in ELISA, maximal binding (E_{MAX}) varied depending on antibody, but was similar in human and mouse cells. These results indicate that the chimeric llama-human antibodies recognize membrane-bound MET in its native conformation in both human and mouse cellular systems. EC₅₀ and E_{MAX} values are shown in Table 10.

TABLE 10
Binding of chimeric mAbs to human and mouse cells as determined by
flow cytometry using increasing concentrations (0-50 nM) of antibodies.

| | Human cells (A549) | | Mouse cells (MLP29) | |
|---|---|---|---|---|
| mAb | EC₅₀ | E_{MAX} | EC₅₀ | E_{MAX} |
| 76H10 | 2.345 | 130.2 | 1.603 | 124.3 |
| 71G3 | 0.296 | 116.9 | 0.214 | 116.2 |
| 71D6 | 0.259 | 112.7 | 0.383 | 121.2 |
| 71C3 | 0.572 | 106.5 | 0.585 | 115.1 |
| 71D4 | 0.371 | 107.2 | 0.498 | 94.8 |
| 71A3 | 0.514 | 160.8 | 0.811 | 144.2 |
| 71G2 | 0.604 | 144.4 | 0.688 | 129.9 |
| 76G7 | 2.298 | 121.2 | 2.371 | 114.8 |
| 71G12 | 2.291 | 109.9 | 2.539 | 121.2 |
| 74C8 | 0.235 | 85.7 | 0.208 | 73.8 |
| 72F8 | 0.371 | 156.3 | 0.359 | 171.6 |

EC₅₀ values are expressed as nMol/L.
E_{MAX} values are expressed as % relative to control.

### Example 4: Receptor Regions Responsible for Antibody Binding

In order to map the receptor regions recognized by antibodies binding to both human and mouse MET (herein after referred to as human/mouse equivalent anti-MET antibodies), we measured their ability to bind to a panel of engineered proteins derived from human MET generated as described (Basilico et al, J Biol. Chem. 283, 21267-21227, 2008). This panel included: the entire MET ECD (Decoy MET); a MET ECD lacking IPT domains 3 and 4 (SEMA-PSI-IPT 1-2); a MET ECD lacking IPT domains 1-4 (SEMA-PSI); the isolated SEMA domain (SEMA); a fragment containing IPT domains 3 and 4 (IPT 3-4). Engineered MET proteins were immobilized in solid phase and exposed to increasing concentrations of chimeric antibodies (0-50 nM) in solution. Binding was revealed using HRP-conjugated anti-human Fc antibodies (Jackson Immuno Research Laboratories). As shown in Table 11, this analysis revealed that 7 mAbs recognize an epitope within the SEMA domain, while the other 4 recognize an epitope within the PSI domain.

TABLE 11
Binding of human/mouse equivalent anti-MET antibodies to the panel
of MET deletion mutants. The MET domain responsible for antibody
binding is indicated in the last column to the right.

| | | SEMA- | | | | |
|---|---|---|---|---|---|---|
| | Decoy | PSI-IPT | SEMA- | | IPT | Binding |
| mAb | MET | 1-2 | PSI | SEMA | 3-4 | domain |
| 76H10 | + | + | + | − | − | PSI |
| 71G3 | + | + | + | − | − | PSI |
| 71D6 | + | + | + | + | − | SEMA |
| 71C3 | + | + | + | + | − | SEMA |
| 71D4 | + | + | + | + | − | SEMA |
| 71A3 | + | + | + | + | − | SEMA |
| 71G2 | + | + | + | + | − | SEMA |
| 76G7 | + | + | + | − | − | PSI |
| 71G12 | + | + | + | − | − | PSI |
| 74C8 | + | + | + | + | − | SEMA |
| 72F8 | + | + | + | + | − | SEMA |

To more finely map the regions of MET responsible for antibody binding, we exploited the absence of cross-reactivity between our antibodies and llama MET (the organism used for generating these immunoglobulins). To this end, we generated a series of llama-human and human-llama chimeric MET proteins spanning the entire MET ECD as described (Basilico et al., J Clin Invest. 124, 3172-3186, 2014). Chimeras were immobilized in solid phase and then exposed to increasing concentrations of mAbs (0-20 nM) in solution. Binding was revealed using HRP-conjugated anti-human Fc antibodies (Jackson Immuno Research Laboratories). This analysis unveiled that 5 SEMA-binding mAbs (71D6, 71C3, 71D4, 71A3, 71G2) recognize an epitope localized between aa 314-372 of human MET, a region that corresponds to blades 4-5 of the 7-bladed SEMA β-propeller (Stamos et al., EMBO J. 23, 2325-2335, 2004). The other 2 SEMA-binding mAbs (74C8, 72F8) recognize an epitope localized between aa 123-223 and 224-311, respectively, corresponding to blades 1-3 and 1-4 of the SEMA β-propeller. The PSI-binding mAbs (76H10, 71G3, 76G7, 71G12) did not appear to display any significant binding to any of the two PSI chimeras. Considering the results presented in Table 11, these antibodies probably recognize an epitope localized between aa 546 and 562 of human MET. These results are summarized in Table 12.

TABLE 12
Mapping of the epitopes recognized by human/mouse equivalent anti-MET antibodies
as determined by ELISA. Human MET ECD (hMET) or llama MET ECD (lMET) as
well as the llama-human MET chimeric proteins (CH1-7) were immobilized
in solid phase and then exposed to increasing concentrations of mAbs.

| | | | | | | | | | | Epitope |
|---|---|---|---|---|---|---|---|---|---|---|
| mAb | hMET | lMET | CH1 | CH2 | CH3 | CH4 | CH5 | CH6 | CH7 | (aa) |
| 76H10 | + | − | + | + | + | + | + | − | − | 546-562 |
| 71G3 | + | − | + | + | + | + | + | − | − | 546-562 |
| 71D6 | + | − | + | + | + | − | − | + | + | 314-372 |
| 71C3 | + | − | + | + | + | − | − | + | + | 314-372 |
| 71D4 | + | − | + | + | + | − | − | + | + | 314-372 |
| 71A3 | + | − | + | + | + | − | − | + | + | 314-372 |
| 71G2 | + | − | + | + | + | − | − | + | + | 314-372 |
| 76G7 | + | − | + | + | + | + | + | − | − | 546-562 |
| 71G12 | + | − | + | + | + | + | + | − | − | 546-562 |
| 74C8 | + | − | + | − | − | − | − | + | + | 123-223 |
| 72F8 | + | − | + | + | − | − | − | + | + | 224-311 |

### Example 5: HGF Competition Assays

The above analysis suggests that the epitopes recognized by some of the human/mouse equivalent anti-MET antibodies may overlap with those engaged by HGF when binding to MET (Stamos et al., EMBO J. 23, 2325-2335, 2004; Merchant et al., Proc Natl Acad Sci USA 110, E2987-2996, 2013; Basilico et al., J Clin Invest. 124, 3172-3186, 2014). To investigate along this line, we tested the competition between mAbs and HGF by ELISA. Recombinant human and mouse HGF (R&D Systems) were biotinylated at the N-terminus using NHS-LC-biotin (Thermo Scientific). MET-Fc protein, either human or mouse (R&D Systems), was immobilized in solid phase and then exposed to 0.3 nM biotinylated HGF, either human or mouse, in the presence of increasing concentrations of antibodies (0-120 nM). HGF binding to MET was revealed using HRP-conjugated streptavidin (Sigma-Aldrich). As shown in Table 13, this analysis allowed to divide human/mouse equivalent anti-MET mAbs into two groups: full HGF competitors (71D6, 71C3, 71D4, 71A3, 71G2), and partial HGF competitors (76H10, 71G3, 76G7, 71G12, 74C8, 72F8).

TABLE 13
Ability of human/mouse equivalent anti-MET antibodies to
compete with HGF for binding to MET as determined by ELISA.

| | hHGF on hMET | | mHGF on mMET | |
|---|---|---|---|---|
| mAb | IC₅₀ (nM) | I_{MAX} (%) | IC₅₀ (nM) | I_{MAX} (%) |
| 76H10 | 1.86 | 64.22 | 2.01 | 62.71 |
| 71G3 | 0.49 | 63.16 | 0.53 | 62.87 |
| 71D6 | 0.29 | 98.34 | 0.34 | 90.54 |
| 71C3 | 1.42 | 93.64 | 1.56 | 89.23 |
| 71D4 | 0.34 | 95.62 | 0.40 | 91.34 |
| 71A3 | 0.51 | 93.37 | 0.54 | 87.74 |
| 71G2 | 0.23 | 97.84 | 0.26 | 91.86 |
| 76G7 | 1.47 | 69.42 | 1.56 | 62.52 |
| 71G12 | 3.87 | 51.39 | 4.05 | 50.67 |
| 74C8 | 0.43 | 76.89 | 0.49 | 71.55 |
| 72F8 | 0.45 | 77.34 | 0.52 | 72.79 |

A MET-Fc chimeric protein (either human or mouse) was immobilized in solid phase and exposed to a fixed concentration of biotinylated HGF (either human or mouse), in the presence of increasing concentrations of antibodies.
HGF binding to MET was revealed using HRP-conjugated streptavidin.
Antibody-HGF competition is expressed as IC₅₀ (the concentration that achieves 50% competition) and I_{MAX} (the maximum % competition reached at saturation).

As a general rule, SEMA binders displaced HGF more effectively than PSI binders. In particular, those antibodies that recognize an epitope within blades 4 and 5 of the SEMA β-propeller were the most potent HGF competitors (71D6, 71C3, 71D4, 71A3, 71G2). This observation is consistent with the notion that SEMA blade 5 contains the high affinity binding site for the α-chain of HGF (Merchant et al., Proc Natl Acad Sci USA 110, E2987-2996, 2013). The PSI domain has not been shown to participate directly with HGF, but it has been suggested to function as a ‘hinge’ regulating the accommodation of HGF between the SEMA domain and the IPT region (Basilico et al., J Clin Invest. 124, 3172-3186, 2014). It is therefore likely that mAbs binding to PSI (76H10, 71G3, 76G7, 71G12) hamper HGF binding to MET by interfering with this process or by steric hindrance, and not by direct competition with the ligand. Finally, blades 1-3 of the SEMA β-propeller have been shown to be responsible for low-affinity binding of the β-chain of HGF, which plays a central role in MET activation but only partially contributes to the HGF-MET binding strength (Stamos et al., EMBO J. 23, 2325-2335, 2004). This could explain why mAbs binding to that region of MET (74C8, 72F8) are partial competitors of HGF.

### Example 6: MET Activation Assays

Due to their bivalent nature, immunoglobulins directed against receptor tyrosine kinases may display receptor agonistic activity, mimicking the effect of natural ligands. To investigate along this line, we tested the ability of human/mouse equivalent anti-MET antibodies to promote MET auto-phosphorylation in a receptor activation assay. A549 human lung carcinoma cells and MLP29 mouse liver precursor cells were deprived of serum growth factors for 48 hours and then stimulated with increasing concentrations (0-5 nM) of antibodies or recombinant HGF (A549 cells, recombinant human HGF, R&D Systems; MLP29 cells, recombinant mouse HGF, R&D Systems). After 15 minutes of stimulation, cells were washed twice with ice-cold phosphate buffered saline (PBS) and then lysed as described (Longati et al., Oncogene 9, 49-57, 1994). Protein lysates were resolved by electrophoresis and then analysed by Western blotting using antibodies specific for the phosphorylated form of MET (tyrosines 1234-1235), regardless of whether human or mouse (Cell Signaling Technology). The same lysates were also analysed by Western blotting using anti-total human MET antibodies (Invitrogen) or anti-total mouse MET antibodies (R&D Systems). This analysis revealed that all human/mouse equivalent antibodies display MET agonistic activity. Some antibodies promoted MET auto-phosphorylation to an extent comparable to that of HGF (71G3, 71D6, 71C3, 71D4, 71A3, 71G2, 74C8). Some others (76H10, 76G7, 71G12, 72F8) were less potent, and this was particularly evident at the lower antibody concentrations. No clear correlation between MET activation activity and HGF-competition activity was observed.

To obtain more quantitative data, the agonistic activity of antibodies was also characterized by phospho-MET ELISA. To this end, A549 and MLP29 cells were serum-starved as above and then stimulated with increasing concentrations (0-25 nM) of mAbs. Recombinant human (A549) or mouse (MLP29) HGF was used as control. Cells were lysed and phospho-MET levels were determined by ELISA as described (Basilico et al., J Clin Invest. 124, 3172-3186, 2014). Briefly, 96 well-plates were coated with mouse anti-human MET antibodies or rat anti-mouse MET antibodies (both from R&D Systems) and then incubated with cell lysates. After washing, captured proteins were incubated with biotin-conjugated anti-phospho-tyrosine antibodies (Thermo Fisher), and binding was revealed using HRP-conjugated streptavidin (Sigma-Aldrich).

The results of this analysis are consistent with the data obtained by Western blotting. As shown in Table 14, 71G3, 71D6, 71C3, 71D4, 71A3, 71G2 and 74C8 potently activated MET, while 76H10, 76G7, 71G12 and 72F8 caused a less pronounced effect. In any case, all antibodies displayed a comparable effect in human and in mouse cells.

TABLE 14
Agonistic activity of human/mouse equivalent anti-MET
antibodies in human and mouse cells as measured by ELISA.

| | A549 cells | | MLP29 cells | |
|---|---|---|---|---|
| mAb | EC₅₀ (nM) | E_{MAX} (%) | EC₅₀ (nM) | E_{MAX} (%) |
| 76H10 | 1.77 | 61.23 | 2.91 | 64.10 |
| 71G3 | 0.41 | 95.72 | 0.37 | 97.81 |
| 71D6 | 0.32 | 101.57 | 0.21 | 114.56 |
| 71C3 | 0.35 | 86.19 | 0.33 | 98.85 |
| 71D4 | 0.59 | 84.63 | 0.51 | 95.34 |
| 71A3 | 0.31 | 86.56 | 0.26 | 95.95 |
| 71G2 | 0.37 | 101.35 | 0.25 | 109.87 |
| 76G7 | 1.86 | 62.34 | 1.19 | 71.45 |
| 71G12 | 2.48 | 70.61 | 2.01 | 75.39 |
| 74C8 | 0.52 | 87.63 | 0.41 | 102.15 |
| 72F8 | 1.51 | 69.74 | 0.79 | 66.82 |
| HGF | 0.19 | 100.00 | 0.23 | 100.00 |

A549 human lung carcinoma cells and MLP29 mouse liver precursor cells were serum-starved and then stimulated with increasing concentrations of mAbs.
Recombinant human HGF (hHGF; A549) or mouse HGF (mHGF; MLP29) was used as control.
Cell lysates were analysed by ELISA using anti-total MET antibodies for capture and anti-phospho-tyrosine antibodies for revealing.
Agonistic activity is expressed as EC₅₀ (nM) and E_{MAX} (% HGF activity).

### Example 7: Scatter Assay

To evaluate whether the agonistic activity of human/mouse equivalent anti-MET antibodies could translate into biological activity, we performed scatter assays with both human and mouse epithelial cells. To this end, HPAF-II human pancreatic adenocarcinoma cells (American Type Culture Collection) and MLP29 mouse liver precursor cells were stimulated with increasing concentrations of recombinant HGF (human or mouse; both from R&D Systems) and cell scattering was determined 24 hours later by microscopy as described previously (Basilico et al., J Clin Invest. 124, 3172-3186, 2014). This preliminary analysis revealed that HGF-induced cell scattering is linear until it reaches saturation at approximately 0.1 nM in both cell lines. Based on these HGF standard curves, we elaborated a scoring system ranging from 0 (total absence of cell scattering in the absence of HGF) to 4 (maximal cell scattering in the presence of 0.1 nM HGF). HPAF-II and MLP29 cells were stimulated with increasing concentrations of human/mouse equivalent anti-MET antibodies, and cell scattering was determined 24 hours later using the scoring system described above. As shown in Table 15, this analysis revealed that all mAbs tested promoted cell scattering in both the human and the mouse cell systems, with substantially overlapping results on both species. 71D6 and 71G2 displayed the very same activity as HGF; 71G3 and 71A3 were just slightly less potent than HGF; 71C3 and 74C8 required a substantially higher concentration in order to match the activity of HGF; 71D4, 76G7, 71G12 and 72F8 did not reach saturation in this assay.

TABLE 15
Biological activity of human/mouse equivalent anti-MET antibodies
as measured in a cell-based scatter assay. HPAF-II human pancreatic
adenocarcinoma cells and MLP29 mouse liver precursor cells were
stimulated with increasing concentrations of human/mouse equivalent
anti-MET antibodies, and cell scattering was determined 24 hours
later using the scoring system described in the text (0, absence
of cell scattering; 4, maximal cell scattering).

| | mAb concentration (nM) | | | | | | | | |
|---|---|---|---|---|---|---|---|---|---|
| mAb | 9.000 | 3.000 | 1.000 | 0.333 | 0.111 | 0.037 | 0.012 | 0.004 | 0.001 |

HPAF-II human pancreatic adenocarcinoma cells

| | | | | | | | | | |
|---|---|---|---|---|---|---|---|---|---|
| 76H10 | 3 | 2 | 1 | 0 | 0 | 0 | 0 | 0 | 0 |
| 71G3 | 4 | 4 | 4 | 4 | 3 | 2 | 1 | 0 | 0 |
| 71D6 | 4 | 4 | 4 | 4 | 4 | 3 | 2 | 1 | 0 |
| 71C3 | 4 | 4 | 3 | 2 | 1 | 0 | 0 | 0 | 0 |
| 71D4 | 2 | 2 | 1 | 0 | 0 | 0 | 0 | 0 | 0 |
| 71A3 | 4 | 4 | 4 | 4 | 3 | 3 | 2 | 0 | 0 |
| 71G2 | 4 | 4 | 4 | 4 | 4 | 3 | 2 | 1 | 0 |
| 76G7 | 3 | 2 | 1 | 0 | 0 | 0 | 0 | 0 | 0 |
| 71G12 | 3 | 2 | 2 | 1 | 0 | 0 | 0 | 0 | 0 |
| 74C8 | 4 | 4 | 3 | 3 | 2 | 1 | 0 | 0 | 0 |
| 72F8 | 3 | 2 | 1 | 0 | 0 | 0 | 0 | 0 | 0 |
| hHGF | 4 | 4 | 4 | 4 | 4 | 3 | 2 | 1 | 0 |
| IgG1 | 0 | 0 | 0 | 0 | 0 | 0 | 0 | 0 | 0 |

MLP29 mouse liver precursor cells

| | | | | | | | | | |
|---|---|---|---|---|---|---|---|---|---|
| 76H10 | 3 | 2 | 1 | 0 | 0 | 0 | 0 | 0 | 0 |
| 71G3 | 4 | 4 | 4 | 4 | 2 | 1 | 0 | 0 | 0 |
| 71D6 | 4 | 4 | 4 | 4 | 4 | 3 | 2 | 1 | 0 |
| 71C3 | 4 | 4 | 3 | 2 | 1 | 0 | 0 | 0 | 0 |
| 71D4 | 2 | 2 | 1 | 0 | 0 | 0 | 0 | 0 | 0 |
| 71A3 | 4 | 4 | 4 | 4 | 3 | 3 | 2 | 0 | 0 |
| 71G2 | 4 | 4 | 4 | 4 | 4 | 2 | 1 | 0 | 0 |
| 76G7 | 3 | 2 | 1 | 0 | 0 | 0 | 0 | 0 | 0 |
| 71G12 | 3 | 2 | 2 | 1 | 0 | 0 | 0 | 0 | 0 |
| 74C8 | 4 | 4 | 3 | 3 | 2 | 1 | 0 | 0 | 0 |
| 72F8 | 3 | 2 | 1 | 0 | 0 | 0 | 0 | 0 | 0 |
| mHGF | 4 | 4 | 4 | 4 | 4 | 3 | 2 | 1 | 0 |
| IgG1 | 0 | 0 | 0 | 0 | 0 | 0 | 0 | 0 | 0 |

### Example 8: Protection Against Drug-Induced Apoptosis

Several lines of experimental evidence indicate that HGF display a potent anti-apoptotic effect on MET-expressing cells (reviewed by Nakamura et al., J Gastroenterol Hepatol. 26 Suppl 1, 188-202, 2011). To test the potential anti-apoptotic activity of human/mouse equivalent anti-MET antibodies, we performed cell-based drug-induced survival assays. MCF10A human mammary epithelial cells (American Type Culture Collection) and MLP29 mouse liver precursor cells were incubated with increasing concentrations of staurosporine (Sigma Aldrich). After 48 hours, cell viability was determined by measuring total ATP concentration using the Cell Titer Glo kit (Promega) with a Victor X4 multilabel plate reader (Perkin Elmer). This preliminary analysis revealed that the drug concentration that induced about 50% cell death is 60 nM for MCF10A cells and 100 nM for MLP29 cells. Next, we incubated MCF10A cells and MLP29 cells with the above determined drug concentrations in the presence of increasing concentrations (0-32 nM) of anti-MET mAbs or recombinant HGF (human or mouse; both from R&D Systems). Cell viability was determined 48 hours later as described above. The results of this analysis, presented in Table 16, suggest that human/mouse equivalent antibodies protected human and mouse cells against staurosporine-induced cell death to a comparable extent. While some mAbs displayed a protective activity similar or superior to that of HGF (71G3, 71D6, 71G2), other molecules displayed only partial protection (76H10, 71C3, 71D4, 71A3, 76G7, 71G12, 74C8, 72F8), either in the human or in the mouse cell system.

TABLE 16
Biological activity of human/mouse equivalent anti-MET
antibodies as measured by a cell-based drug-induced apoptosis assay.

| | MCF10A cells | | MLP29 cells | |
|---|---|---|---|---|
| mAb | EC₅₀ (nM) | E_{MAX} (%) | EC₅₀ (nM) | E_{MAX} (%) |
| 76H10 | >32.00 | 22.75 | >32.00 | 27.21 |
| 71G3 | 5.04 | 65.23 | 4.85 | 62.28 |
| 71D6 | 1.48 | 66.81 | 0.95 | 68.33 |
| 71C3 | 31.87 | 50.16 | 31.03 | 51.32 |
| 71D4 | 30.16 | 51.71 | 29.84 | 52.13 |
| 71A3 | <0.50 | 71.70 | <0.50 | 70.54 |
| 71G2 | 1.06 | 64.85 | 1.99 | 58.29 |
| 76G7 | 25.41 | 51.93 | 30.08 | 50.16 |
| 71G12 | >32.00 | 39.35 | >32.00 | 39.73 |
| 74C8 | >32.00 | 41.74 | >32.00 | 37.52 |
| 72F8 | >32.00 | 35.79 | >32.00 | 43.81 |
| HGF | 4.57 | 59.28 | 5.35 | 58.65 |

MCF10A human mammary epithelial cells and MLP29 mouse liver precursor cells were incubated with a fixed concentration of staurosporine in the the presence of increasing concentrations of anti-MET mAbs or recombinant HGF (human or mouse), and total ATP content was determined 48 hours later.
Cell viability was calculated as % total ATP content relative to cells treated with neither staurosporine nor antibodies, and is expressed as EC₅₀ and E_{MAX}.

### Example 9: Branching Morphogenesis Assay

HGF is a pleiotropic cytokine which promotes the harmonic regulation of independent biological activities, including cell proliferation, motility, invasion, differentiation and survival. The cell-based assay that better recapitulates all of these activities is the branching morphogenesis assay, which replicates the formation of tubular organs and glands during embryogenesis (reviewed by Rosario and Birchmeier, Trends Cell Biol. 13, 328-335, 2003). In this assay, a spheroid of epithelial cells is seeded inside a 3D collagen matrix and is stimulated by HGF to sprout tubules which eventually form branched structures. These branched tubules resemble the hollow structures of epithelial glands, e.g. the mammary gland, in that they display a lumen surrounded by polarized cells. This assay is the most complete HGF assay that can be run in vitro.

In order to test whether human/mouse equivalent anti-MET antibodies displayed agonistic activity in this assay, we seeded LOC human kidney epithelial cells (Michieli et al. Nat Biotechnol. 20, 488-495, 2002) and MLP29 mouse liver precursor cells in a collagen layer as described (Hultberg et al., Cancer Res. 75, 3373-3383, 2015), and then exposed them to increasing concentrations of mAbs or recombinant HGF (human or mouse, both from R&D Systems). Branching morphogenesis was followed over time by microscopy, and colonies were photographed after 5 days. Quantification of branching morphogenesis activity was obtained by counting the number of branches for each spheroid. As shown in Table 17, all antibodies tested induced dose-dependent formation of branched tubules. However, consistent with the data obtained in MET auto-phosphorylation assays and cell scattering assays, 71D6, 71A3 and 71G2 displayed the most potent agonistic activity, similar or superior to that of recombinant HGF.

TABLE 17
Branching morphogenesis assay.

| | | | | |
|---|---|---|---|---|
| mAb | 0 nM | 0.5 nM | 2.5 nM | 12.5 nM |

LOC cells

| | | | | |
|---|---|---|---|---|
| 76H10 | 3.3 ± 1.5 | 7.3 ± 0.6 | 11.7 ± 1.5 | 16.7 ± 1.5 |
| 71G3 | 3.0 ± 1.0 | 13.7 ± 1.5 | 19.0 ± 2.6 | 22.3 ± 2.1 |
| 71D6 | 3.0 ± 1.0 | 29.0 ± 2.0 | 29.0 ± 2.6 | 32.7 ± 1.5 |
| 71C3 | 3.3 ± 0.6 | 8.7 ± 1.5 | 12.7 ± 2.1 | 15.7 ± 2.1 |
| 71D4 | 3.0 ± 1.0 | 9.0 ± 2.6 | 15.7 ± 1.2 | 18.7 ± 1.5 |
| 71A3 | 3.0 ± 1.7 | 24.0 ± 4.6 | 30.3 ± 3.2 | 31.3 ± 1.5 |
| 71G2 | 3.7 ± 1.5 | 25.3 ± 2.1 | 29.3 ± 3.5 | 31.7 ± 3.5 |
| 76G7 | 2.7 ± 0.6 | 6.7 ± 0.6 | 13.3 ± 4.2 | 16.3 ± 5.7 |
| 71G12 | 3.3 ± 0.6 | 7.0 ± 2.6 | 15.3 ± 5.5 | 16.0 ± 4.6 |
| 74C8 | 3.0 ± 1.0 | 10.3 ± 4.2 | 17.0 ± 4.6 | 18.7 ± 4.9 |
| 72F8 | 3.3 ± 1.5 | 9.0 ± 3.5 | 12.3 ± 2.1 | 16.0 ± 3.0 |
| hHGF | 3.0 ± 1.0 | 18.0 ± 2 | 27.7 ± 2.5 | 20.3 ± 2.1 |

MLP29 cells

| | | | | |
|---|---|---|---|---|
| 76H10 | 0.3 ± 0.6 | 10.7 ± 4.0 | 14.3 ± 3.2 | 24.7 ± 6.0 |
| 71G3 | 0.3 ± 0.6 | 24.7 ± 4.5 | 34.3 ± 5.5 | 29.3 ± 8.0 |
| 71D6 | 1.3 ± 1.2 | 32.7 ± 3.5 | 39.0 ± 7.5 | 41.3 ± 8.0 |
| 71C3 | 0.3 ± 0.6 | 11.7 ± 3.5 | 15.7 ± 6.5 | 24.7 ± 6.5 |
| 71D4 | 0.7 ± 1.2 | 16.0 ± 2.6 | 14.7 ± 4.5 | 21.7 ± 5.5 |
| 71A3 | 0.7 ± 0.6 | 30.3 ± 2.1 | 42.0 ± 6.2 | 42.7 ± 8.0 |
| 71G2 | 1.0 ± 1.0 | 34.0 ± 2.6 | 46.3 ± 4.7 | 45.0 ± 7.0 |
| 76G7 | 0.3 ± 0.6 | 14.7 ± 2.1 | 18.7 ± 4.5 | 24.7 ± 6.5 |
| 71G12 | 1.0 ± 1.0 | 14.0 ± 2.6 | 14.7 ± 5.5 | 22.7 ± 6.0 |
| 74C8 | 0.7 ± 0.6 | 17.3 ± 2.5 | 15.3 ± 6.0 | 22.3 ± 9.0 |
| 72F8 | 1.0 ± 1.0 | 12.7 ± 3.1 | 11.7 ± 3.5 | 18.7 ± 2.5 |
| mHGF | 0.7 ± 1.2 | 32.3 ± 4.0 | 43.7 ± 4.2 | 36.0 ± 7.2 |

Cell spheroids preparations of LOC human kidney epithelial cells or MLP29 mouse liver precursor cells were seeded in a collagen layer and then incubated with increasing concentrations (0, 0.5, 2.5 and 12.5 nM) of mAbs or recombinant HGF (LOC, human HGF; MLP29, mouse HGF).
Branching morphogenesis was followed over time by microscopy, and colonies were photographed after 5 days.
Branching was quantified by counting the number of branches for each spheroid (primary branches plus secondary branches).

### Example 10: Fine Epitope Mapping

In order to finely map the epitopes of MET recognized by human/mouse equivalent anti-MET antibodies we pursued the following strategy. We reasoned that, if an antibody generated in llamas and directed against human MET cross-reacts with mouse MET, then this antibody probably recognizes a residue (or several residues) that is (or are) conserved between H. sapiens and M. musculus but not among H. sapiens, M. musculus and L. glama. The same reasoning can be extended to R. norvegicus and M. fascicularis.

To investigate along this line, we aligned and compared the amino acid sequences of human (UniProtKB #P08581; aa 1-932), mouse (UniProtKB #P16056.1; aa 1-931), rat (NCBI #NP_113705.1; aa 1-931), cynomolgus monkey (NCBI #XP_005550635.2; aa 1-948) and llama MET (GenBank #KF042853.1; aa 1-931) among each other. With reference to Table 12, we concentrated our attention within the regions of MET responsible for binding to the 71D6, 71C3, 71D4, 71A3 and 71G2 antibodies (aa 314-372 of human MET) and to the 76H10 and 71G3 antibodies (aa 546-562 of human MET). Within the former region of human MET (aa 314-372) there are five residues that are conserved in human and mouse MET but not in llama MET (Ala 327, Ser 336, Phe 343, Ile 367, Asp 372). Of these, four residues are also conserved in rat and cynomolgus monkey MET (Ala 327, Ser 336, Ile 367, Asp 372). Within the latter region of human MET (aa 546-562) there are three residues that are conserved in human and mouse MET but not in llama MET (Arg 547, Ser 553, Thr 555). Of these, two residues are also conserved in rat and cynomolgus monkey MET (Ser 553 and Thr 555).

Using human MET as a template, we mutagenized each of these residues in different permutations, generating a series of MET mutants that are fully human except for specific residues, which are llama. Next, we tested the affinity of selected SEMA-binding mAbs (71D6, 71C3, 71D4, 71A3, 71G2) and PSI-binding mAbs (76H10 and 71G3) for these MET mutants by ELISA. To this end, the various MET proteins were immobilized in solid phase (100 ng/well in a 96-well plate) and then exposed to increasing concentrations of antibodies (0-50 nM) solution. As the antibodies used were in their human constant region format, binding was revealed using HRP-conjugated anti-human Fc secondary antibody (Jackson Immuno Research Laboratories). Wild-type human MET was used as positive control. The results of this analysis are presented in Table 18.

TABLE 18
The epitopes of MET responsible for agonistic antibody binding
represent residues conserved among H. sapiens, M. musculus,
R. norvegicus, M. fascicularis but not among the same species
and L. glama. The relevance of residues conserved among
human, mouse, rat, cynomolgus monkey but not llama MET for
binding to agonistic mAbs was tested by ELISA. Wild-type
(WT) or mutant (MT) human MET ECD was immobilized in solid
phase and exposed to increasing concentrations of mAbs in
solution. Binding was revealed using anti-human Fc secondary
antibodies. All binding values were normalized to the WT protein
and are expressed as % binding (E_{MAX}) compared to WT MET.

| | | mAb binding (% WT MET ECD) | | | | | | |
|---|---|---|---|---|---|---|---|---|
| | MUTA- | SEMA BINDERS | | | | | PSI BINDERS | |
| MT | TIONS | 71D6 | 71C3 | 71D4 | 71A3 | 71G2 | 76H10 | 71G3 |
| WT | — | 100.0 | 100.0 | 100.0 | 100.0 | 100.0 | — | — |
| A | 1, 2, 3 | 103.3 | 99.8 | 114.5 | 116.8 | 92.1 | — | — |
| B | 4, 5 | 0.0 | 0.0 | 0.0 | 0.0 | 0.0 | — | — |
| C | 1, 2, 3, 4, 5 | 0.0 | 0.0 | 0.0 | 0.0 | 0.0 | — | — |
| D | 1, 2 | 128.0 | 101.8 | 119.6 | 127.9 | 113.5 | — | — |
| E | 2, 3, 4 | 43.6 | 59.6 | 57.2 | 65.4 | 41.4 | — | — |
| F | 2, 4, 5 | 0.0 | 0.0 | 0.0 | 0.0 | 0.0 | — | — |
| G | 3, 4, 5 | 0.0 | 0.0 | 0.0 | 0.0 | 0.0 | — | — |
| H | 2, 4 | 38.6 | 61.6 | 58.7 | 76.7 | 40.2 | — | — |
| I | 6, 7, 8 | — | — | — | — | — | 100.0 | 100.0 |
| J | 6, 7 | — | — | — | — | — | 89.0 | 91.2 |
| K | 6, 8 | — | — | — | — | — | 0.0 | 0.0 |
| L | 7, 8 | — | — | — | — | — | 0.0 | 0.0 |

The results presented above provide a definite and clear picture of the residues relevant for binding to our agonistic antibodies.

All the SEMA binders tested (71D6, 71C3, 71D4, 71A3, 71G2) appear to bind to an epitope that contains 2 key amino acids conserved in human, mouse, cynomolgus and rat MET but not in llama MET lying within blade 5 of the SEMA β-propeller: Ile 367 and Asp 372. In fact, mutation of Ala 327, Ser 336 or Phe 343 did not affect binding at all; mutation of Ile 367 partially impaired binding; mutation of Ile 367 and Asp 372 completely abrogated binding. We conclude that both Ile 367 and Asp 372 of human MET are important for binding to the SEMA-directed antibodies tested.

Also the PSI binders tested (76H10, 71G3) appear to bind to a similar or the same epitope. In contrast to the SEMA epitope, however, the PSI epitope contains only one key amino acid also conserved in human, mouse, cynomolgus and rat MET but not in llama MET: Thr 555. In fact, mutation of Arg 547 or Ser 553 did not affect binding at all, while mutation of Thr 555 completely abrogated it. We conclude that Thr 555 represents the crucial determinant for binding to the PSI-directed antibodies tested.

### Example 11: MET Agonist Antibodies Promote Langerhans Islet Growth and Pancreatic Beta Cell Regeneration in Healthy Mice

In order to assess the biological effect of a MET agonistic antibody on pancreatic beta cells in vivo, we subjected both male and female adult BALB/c mice (Charles River) to systemic treatment with 0, 3, 10 or 30 mg/kg purified 71D6 antibody for a period of three months (6 mice per gender per group for a total of 48 animals). Antibody was administered 2 times a week by i.p. injection. Body weight and fasting blood glucose concentration was measured every month throughout the experiment. At the end of the 3 month period, mice were sacrificed; pancreas were collected, embedded in paraffin and processed for histological analysis. Sections were stained with hematoxylin and eosin, examined by microscopy and photographed. Images were analyzed using ImageJ software (National Institutes of Health) to determine Langerhans islet number and size.

Chronic treatment with 71D6 did not affect total body weight in either male or female animals (FIG. 1A). Likewise, basal glycemia measured in fasting animals did not change at any antibody dose (FIG. 1B). On the other hand, histological analysis of pancreatic sections revealed that treatment with 71D6 agonistic antibody significantly increased the number of Langerhans islets in a dose-dependent fashion (FIG. 2A). In untreated, control animals (0 mg/kg), the number of islets per unit of pancreas section (mm²) was approximately 3. At the maximal dose tested (30 mg/kg), islet number per mm² reached a value of 6; at 3 and 10 mg/kg islet density displayed intermediate values. Treatment with 71D6 also significantly increased Langerhans islet size (FIG. 2B). In control animals, the mean islet size was approximately 0.01 mm² (expressed as the area of the islet section, as measured by microscopic imaging on tissue section stained with hematoxylin and eosin). At a dose of 3 mg/kg, mean islet area increased 2 times compared to 0 mg/kg; at a dose of 10 mg/kg, it increased 3 times compared to control; at 30 mg/kg, islets were 4 times bigger compared to untreated animals. Representative images of pancreas sections stained with hematoxylin and eosin are shown in FIG. 2C.

Interestingly, immunohistochemical analysis with anti-insulin antibodies revealed that treatment with 71D6 results in expansion of the pancreatic beta cell population and in potentiation of insulin expression (FIG. 3). This finding suggests that 71D6-induced size increase of Langerhans islets is due to hyperproliferation of pancreatic beta cells. Furthermore, potentiated insulin expression demonstrates that these beta cells are healthy and functional. Altogether, these results indicate that 71D6 acts as a mitogenic and pro-regenerative factor for pancreatic beta cells in vivo.

### Example 12: MET Agonist Antibodies Promote Langerhans Islet Growth and Pancreatic Beta Cell Regeneration in a Mouse Model of Type 1 Diabetes Mellitus

Prompted by the observation that agonistic anti-MET antibodies act as mitogenic factors for beta cells, we tested their therapeutic potential in a mouse model of type 1 diabetes. Ablation of pancreatic beta cells was achieved in mice by administration of multiple, low doses of streptozotocin (STZ; a chemical agent that selectively kills beta cells and a standard compound used to induce type 1 diabetes mellitus in laboratory animals).

STZ was injected i.p. into female BALB-c mice (Charles River) at a dose of 40 mg/kg every 24 hours for 5 consecutive days. One week after the last injection, STZ-treated mice displayed a mean basal glycemia two times higher compared to untreated mice (240 mg/dL vs. 120 mg/dL), suggesting that the chemical compound had efficiently killed beta cells. At this point, mice were randomized into 4 arms of 7 mice each based on basal glycemia, which received treatment with (i) vehicle only (PBS), (ii) purified 71D6 antibody, (iii) purified 71G2 antibody, (iv) purified 71G3 antibody. Antibodies were administered at a dose of 1 mg/kg two times a week by i.p. injection. An additional, fifth arm contained 7 mice that received no STZ or antibody and served as a healthy control. The experiment continued for 8 weeks; basal glycemia was monitored throughout the experiment. At the end of the 8 week period, mice were sacrificed and subjected to autopsy. Blood was collected for analysis; pancreases were extracted, processed for histology and embedded in paraffin.

As shown in FIG. 4A, basal blood glucose levels in STZ-treated mice continued to increase over time. This is consistent with the notion that STZ-induced beta cell damage causes chronic pancreas inflammation, leading to progressive aggravation of organ injury. Interestingly, antibody administration did not completely normalize glycemia, but significantly lowered it towards more normal levels. Six weeks after treatment start (i.e. 7 weeks after last STZ injection), mice treated with STZ only displayed a mean basal glycemia of approximately 250 mg/dL; mice treated with STZ and 71D6 had a mean basal glycemia of approximately 150 mg/dL; mice treated with STZ and 71G2 or 71G3 displayed a slightly higher glycemia, but still significantly lower than the STZ alone arm; control untreated mice showed a mean basal glycemia of 96 mg/dL (FIG. 4B).

In order to determine the effect of MET agonist antibodies on Langerhans islets, pancreas sections were stained with hematoxylin and eosin and analysed by microscopy. Digital images of Langerhans islets were analysed using ImageJ software (National Institutes of Health). The number, density and size of Langerhans islets were determined by digital data analysis. As shown in FIG. 5A, STZ administration dramatically reduced the number of Langerhans islets in the pancreas of mice treated with this compound only. In contrast, animals treated with STZ and 71D6 displayed a more normal Langerhans islet density, very similar to that observed in untreated, control mice. STZ treatment also heavily affected Langerhans islet size, reducing it by more than 6 times (FIG. 5B). Remarkably, 71D6 antagonized this reduction, limiting it to 1.5 times. Similar results were obtained with 71G2 and 71G3, although with similar but slightly reduced potency (71D6>71G2>71G3). Representative images of pancreas sections stained with hematoxylin and eosin are shown in FIG. 5C.

Pancreas sections were further analysed by immunohistochemistry using anti-insulin antibodies. This analysis revealed that STZ not only reduced Langerhans islet number and size, but also dramatically curtailed beta cells and, as a consequence, insulin production. Again notably, MET agonist antibody treatment rescued beta cells from STZ-induced destruction and maintained insulin production elevated. This may explain the lower levels of blood glucose observed in animals treated with both STZ and MET agonist antibodies compared to mice receiving STZ only. Representative images of pancreas sections stained with anti-insulin antibodies are shown in FIG. 6.

### Example 13: MET Agonist Antibodies Promote Langerhans Islet Growth and Pancreatic Beta Cell Regeneration in a Mouse Model of Type 2 Diabetes Mellitus

Prompted by the observation that anti-MET agonistic antibodies induce pancreatic beta cells regeneration in healthy mice and in a type 1 diabetes mellitus model, we set to test their therapeutic potential further in other related indications. Although characterized by different etiological mechanisms, type 2 diabetes also leads to Langerhans islet degeneration. In fact, type 2 diabetes is characterized by hyperinsulinaemia in the presence of insulin resistance, leading to high blood glucose levels and inability of beta cells to compensate for the increased demand of insulin (Christoffersen et al., Am J Physiol Regul lntegr Comp Physiol 297:1195-201, 2009). Therefore, regeneration of beta cells is also an unmet medical need for type 2 diabetes mellitus patients.

In order to explore the therapeutic potential of agonistic MET antibodies in type 2 diabetes, we selected the db/db obese mouse model. Due to a mutation in the leptin gene, these animals are hyperphagic, obese, hyperinsulinemic and hyperglycemic. Obesity is evident from 3-4 weeks of age, with hyperinsulinemia becoming apparent at around week 2 and hyperglycaemia developing between weeks 4 and 8. Female db/db mice were obtained from Charles River at the age of 7 weeks. One week later, animals were randomized into 4 arms of five mice each, which received treatment with (i) vehicle only (PBS), (ii) purified 71D6 antibody, (iii) purified 71G2 antibody, (iv) purified 71G3 antibody. Antibodies were administered at a dose of 1 mg/kg two times a week by i.p. injection. Considering that the background strain of db/db mice is C57BL6/J, we used these mice as healthy control animals. Basal glycemia was monitored throughout the experiment. After 8 weeks of treatment (16 weeks of age), mice were sacrificed and subjected to autopsy. Pancreases were collected, processed for histology and embedded in paraffin. Tissue sections were stained with hematoxylin and eosin in order to visualize Langerhans islets. Beta cells and insulin production were highlighted by immunohistochemical analysis using anti-insulin antibodies.

As shown in FIG. 7A, untreated db/db mice already displayed a fairly advanced hyperglycemia at week 7 of age (approximately 240 mg/dL). Thereafter, blood glucose levels steadily increased until they reached a plateau exceeding 300 mg/dL. Interestingly, animals treated with 71D6, 71G2 and 71G3 displayed a significantly lower glycemia throughout the experiment, although not matching that of control C57BL6/J control mice. At the end of the experiment, db/db untreated mice had a basal glycemia of approximately 330 mg/dL; 71D6-treated db/db animals showed instead a mean basal glycemia of approximately 140 mg/dL; 71G2- and 71G3-treated animals displayed a basal glycemia of approximately 180 mg/dL (FIG. 7B).

Pancreas sections stained with hematoxylin and eosin were analysed by microscopy and photographed. Langerhans islets were analysed using ImageJ software to estimate islet number, density and size. This analysis revealed that Langerhans islets are extremely degenerated in db/db mice at 16 weeks of age compared to age-matching C57BL6/J controls, both in terms of number and size. In fact, C57BL6/J mice displayed a mean pancreatic islet density of 2.3 islets/mm², while untreated db/db mice showed a density of 1.6 islets/mm² (FIG. 8A). Strikingly, islet density dramatically increased in db/db mice treated with 71D6, reaching values significantly higher than those observed in healthy controls (4.4 islets/mm²). Islet size was also heavily impaired in db/db mice (FIG. 8B) compared to C57BL6/J controls. In the latter strain, Langerhans islets had a mean area of 0.3 mm², which was reduced by approximately 10 times in untreated db/db mice. Strikingly, 71D6 treatment completely rescued islet size decrease, bringing it back to values similar or even greater than those characteristic of C57BL6/J healthy animals. Similar results with respect to both islet number and size were obtained with 71G2 and 71G3, although with slightly reduced potency (71D6>71G2>71G3). Representative images of pancreas sections stained with hematoxylin and eosin are shown in FIG. 8C.

We further characterized the biological effects of 71D6 by assessing its ability to specifically affect the beta cell population. To this end, pancreas sections were analysed by immunohistochemistry using anti-insulin antibodies. This analysis revealed that the few surviving islets in db/db mice contained very few insulin-expressing beta cells compared to healthy controls (FIG. 9). In contrast, db/db mice treated with 71D6, 71G2 or 71G3 contained significantly more functional beta cells, and these cells expressed much higher levels of insulin. This was particularly evident in the 71D6 arm, confirming that this antibody is more potent than 71G2 and 71G3.

These results as well as those presented in the previous Examples demonstrate that the 71D6, 71G2 and 71G3 MET agonistic antibodies promote beta cell survival and regeneration, contributing to maintaining normal levels of insulin. Considering that restoring functional beta cells significantly improves the symptoms of diabetes and the quality of life of diabetes patients, we suggest that agonistic anti-MET antibodies could represent an innovative tool for diabetes treatment in the clinic.

Importantly, a key requisite for moving MET agonistic antibodies forward to the clinic is their complete cross-reactivity with pre-clinical species, including rodents and non-human primates. In fact, we were able to demonstrate therapeutic activity of 71D6, 71G2 and 71G3 in mice because they maintain full cross-reactivity between human and mouse MET. Furthermore, 71D6 elicits exactly the same biological activity and potency in tissues of human, mouse, rat and monkey origin. Without this species equivalency it would be impossible to move the described MET agonist antibodies on towards first-in-human experimentation. Mainly due to this reason (i.e. absence of equivalency in preclinical species), the any agonistic MET antibodies known in the prior art could not be tested in preclinical models and lack therefore the necessary proof-of-efficacy.

Further along this avenue, another approach to treat both type 1 and type 2 diabetes mellitus is represented by pancreas transplantation, either as a whole organ or using isolated Langerhans islets or purified beta cells (Kieffer et al., J Diabetes Investig. 2017, epub ahead of print; doi: 10.1111/jdi.12758). This approach also has some limitations, particularly with respect to poor grafting and scarce survival of transplanted beta cells in the recipient. Given the potent ability of MET agonist antibodies described herein to promote beta cell regeneration and insulin secretion, they may also improve the efficacy of pancreatic tissue transplantation and amplify the beta cell population in graft-receiving patients.

### Example 14: MET Agonist Antibodies Preserve Pancreatic Beta Cell Function, Prevent Diabetes Onset and Cooperate with Immune-Suppressing Drugs in a Mouse Model of Autoimmune Type 1 Diabetes Mellitus

Type 1 diabetes mellitus is characterized by autoimmune-mediated destruction of pancreatic beta cells, leading to insufficient insulin secretion and inability of tissues to uptake blood glucose. Auto-antibody-mediated beta cell destruction begins earlier than the hyperglycemic phenotype manifests. At the time insulin-dependent diabetes is diagnosed, typically during adolescence, beta cell destruction may be already advanced, with only a minor fraction of the original beta cells surviving. Furthermore, beta cell destruction proceeds very rapidly, thus leaving a narrow window for therapeutic intervention after diagnosis.

Immuno-suppressive drugs are being investigated as therapy for newly-diagnosed type 1 diabetes patients, in an effort to reduce autoimmune-mediated islet cell destruction. However, immunosuppressants require several months before showing the first clinical benefits. When this occurs, approximately half year after treatment start, the beta cells of the pancreas continue to be destroyed, often completely. As a result, the efficacy of immunosuppressants is severely blunted if not nullified. Maintaining islet beta cells alive—or even better regenerating them—during this crucial window is a highly unmet medical need for diabetes patients.

In order to test whether MET-agonistic antibodies could antagonize immune-mediated beta cell destruction and cooperate with immune-targeting drugs in the context of type 1 diabetes, we selected an appropriate mouse model. NOD/ShiLtJ strain (commonly called NOD) is a polygenic model for autoimmune type 1 diabetes. Diabetes in NOD mice is characterized by hyperglycemia and leukocytic infiltration of the pancreatic islets. Marked decreases in pancreatic insulin content occur in females at about 12 weeks of age and several weeks later in males. NOD mice are considered the type 1 diabetes animal model that best reproduces the pathology observed in humans. In this strain, several studies have been conducted with immunosuppressants for studying their potential in ameliorating hyperglycemia and/or delaying diabetes onset. In particular, antibodies directed against the lymphocyte-specific surface marker CD3 have been shown to be particularly effective in several studies (Chatenoud et al. Proc Natl Acad Sci USA 91:123-127, 1994; Chatenoud et al. J Immunol 158:2947-2954, 1997; Gill et al. Diabetes 65:1310-1316, 2016; Kuhn et al. Immunotherapy, 8:889-906, 2016; Kuhn et al. J Autoimmun 76:115-122, 2017). Interestingly, these studies showed that oral delivery of these immune-targeted antibodies gives rise to less side effects compared to systemic delivery. The most effective protocol consisted in treating mice for 5 consecutive days and then stopping the therapy (Ochi et al. Nat Med. 12:627-635, 2006). Notably, the therapeutic efficacy dramatically dropped when the oral drug dose exceeded 5 μg per mouse (0.25 mg/kg).

To test whether our agonistic anti-MET antibodies displayed a therapeutic effect and to investigate their potential cooperation with immune-targeting drugs, we obtained seventy-two 6-week-old female NOD mice from Charles River. Blood sugar was measured in random fed (i.e. not fasting) animals using test strips for human use (multiCare in; Biochemical Systems International). At this time, NOD mice displayed a pre-diabetic, average glycemia of approximately 110 mg/dL (FIG. 10A). Mice were randomized into four different arms of 18 animals each, making sure that all groups were as homogeneous as possible with respect to glycemia. Starting from week 7, the four arms were subjected to different treatments as follows: no drug (CONTROL); 0.15 mg/kg anti-CD3 antibody (CD3); 3 mg/kg purified 71D6 antibody (71D6), 0.15 mg/kg anti-CD3 antibody+3 mg/kg purified 71D6 antibody (COMBO). Anti-CD3 antibody was delivered orally by gavage in 100 μL of PBS one time per day for 5 consecutive days and then interrupted as per protocol. 71D6 was delivered i.p. in 200 μl of PBS two times per week for the whole duration of the experiment. Mice were fed ad libitum with a standard diet. Glycemia was measured on random fed animals one time per week using strips as above. An animal was considered diabetic if it showed a glycemic value greater than 250 mg/dL for 2 consecutive weeks.

In line with the literature, no diabetic animal was recorded until week 12 (FIG. 10B). At week 13, diabetes started manifesting in the CONTROL and CD3 arms. At week 18, 50% of the CONTROL animals were diabetic (FIG. 10C), exactly as described by the original strain provider (The Jackson Lab—001976 mouse strain datasheet; https://www.jax.org/strain/001976). At week 21, when the experiment was interrupted, 88% of the CONTROL mice were diabetic, while the other arms displayed significant lower values: CD3, 47%; 71D6, 21%; COMBO, 14% (FIG. 10D). Analysis of diabetes onset over time is shown in FIG. 11A. A Kaplan-Meier plot is shown in FIG. 11B. Statistical analysis was performed using Prism software (Graph Pad). A Mantel-Cox test, a Logrank test for trend and a Gehan-Breslow-Wilcoxon test all gave a p value of less than 0.001, indicating that the differences among curves are statistically significant.

Average non-fasting glycemia increased constantly in all arms, but reached extremely high levels (>450 mg/dL) only in the CONTROL untreated arm (FIG. 12). Consistent with the diabetes onset data, blood sugar levels followed a precise order: CONTROL>CD3>71D6>COMBO. During the course of the experiment (4 months), a few mice died for reasons independent of treatment, mainly in-cage fighting with fellow mice and bacterial infections (CONTROL, 1/18; CD3, 1/18; 71D6, 4/18; COMBO, 4/14). Since glycemia levels in single diabetic mice reached quickly extreme values (>550 mg/dL), animals were sacrificed three weeks after diabetes diagnosis. In these cases, a value of 550 mg/dL was used for computing the average glycemia of a group even after death. All mice, whether diabetic or not, where sacrificed at the end of week 21.

Before sacrifice, all mice were subjected to a glucose tolerance test (GTT). To this end, animals were food-starved overnight. The morning after, a blood sample was collected for glycemia and insulin measurement. A glucose solution (3 g/kg in 200 μL PBS) was injected i.p. and a second blood sample was collected 3 minutes later. Soon after, mice were sacrificed and the major organs were collected for analysis, including the liver and the pancreas. Blood glucose concentration was determined using strips as described above. Insulin concentration was measured with an Ultra-Sensitive Mouse Insulin ELISA Kit (Crystal Chem).

Blood sugar content analysis revealed the following scenario. At time zero, glycemia was lower in the treated arms compared to control (CONTROL>CD3>71D6>COMBO; FIG. 13A), but three minutes after glucose challenging it raised at similar levels in all groups (>350 mg/dL; FIG. 13B). In contrast, blood insulin concentration was very low at time zero, with the exception of the COMBO arm that showed slightly higher levels (FIG. 13C). Notably, following glucose injection, insulin levels appeared very different depending on the treatment arm, showing a reverse order (COMBO>71D6>CD3>CONTROL; FIG. 13D). Since NOD mice display a peculiar modulation of insulin in their pre-diabetic stage (Amrani et al. Endocrinology 139:1115-1124, 1998), it is difficult to directly compare these absolute values with other non-diabetic strains. In any case, we can certainly conclude that animals belonging to the treatment arms respond to glucose stimulation by secreting insulin, while control animals do not.

Consistent with an ameliorated diabetic phenotype, body weight was slightly (although not significantly) higher in the treatment arms compared to the control arm at the time of autopsy (FIG. 14A). There was no significant difference in liver to body weight in any of the group (FIG. 14B), suggesting that 71D6-mediated liver growth (observed in other mouse systems) is strain-specific. No other biological or pathological sign or tract was detected in 71D6-treated animals while performing autopsy or when analyzing tissue histology.

Pancreas samples were embedded in paraffin and processed for histological analysis. Tissue section were stained with hematoxylin and eosin and analyzed by microscopy. This analysis revealed that the pancreas of the majority of animals belonging to the CONTROL arm contained a very small number of Langerhans islets, and those islets that were visible were abnormally small and highly infiltrated with lymphocytes (FIG. 15). In contrast, Langerhans islets in the CD3 arm were more abundant and less degenerated, although still infiltrated with lymphocytic cells. Pancreas sections of the 71D6 arms contained more Langerhans islets compared to both CONTROL and CD3, and islet size was greater on average; however, lymphocyte infiltration was still evident. Finally, pancreatic islets of the COMBO arm were abundant and big, although infiltrated as well.

The major treatment-dependent differences were observed in pancreas sections stained with anti-insulin antibodies (FIG. 16). In the CONTROL arm, very little if any staining was observed in the few visible islets. In the CD3 arm, insulin signal was higher, although not as potent as observed in 71D6-treated animals. Islets found in the COMBO arm displayed the highest and most homogeneous insulin signal compared to all other arms. At higher magnification, these features could be appreciated in greater detail (FIG. 17). Islets in untreated animals contained very few if any insulin-producing cell. In contrast, the majority of islet cells in the CD3 arm were positive for insulin. In the 71D6 arm, islets were both large and intensively stained. Pancreas of the COMBO arm contained the largest and most insulin-producing islets among all groups.

As mentioned above, the number of insulin-producing beta cells within the Langerhans islets was clearly higher in the treated arms (COMBO>71D6>CD3>CONTROL). However, cell infiltration was very dishomogeneous, and no major differences with respect to the number of lymphocytic cells recruited around the islets could be observed among the various arms. This could be explained by two different mechanisms depending on the therapeutic agent. It is well established that oral delivery of anti-CD3 antibodies induces immunogenic tolerance rather than eliminating the immune response (Chatenoud et al. J Immunol 158:2947-2954, 1997). The tolerogenic process involves activation and proliferation of T-regulatory cells, which inhibit auto-antibody-mediated beta cell destruction (Chatenoud Novartis Found Symp 252:279-220, 2003). This explains why, in the CD3 arm, pancreatic beta cells are not destroyed in spite of immune cell infiltration. On the other hand, the data presented in the previous examples suggest that 71D6 promotes beta cell survival and regeneration. It can be therefore hypothesized that 71D6 both antagonizes immune-mediated beta cell death and promote beta cell growth, thus preserving beta cell mass despite heavy immune cell infiltration.

To further investigate the role of the immune system in the response to anti-CD3 and anti-MET antibodies, we measured anti-insulin antibodies in mouse plasma. To this end, plasma samples collected at autopsy from all mice as well as from young, pre-diabetic female NOD mice (week 7 of life) were analyzed using a Mouse IAA (Insulin Auto-Antibodies) ELISA Kit (Fine Test). This analysis revealed that most mice displayed high concentrations of anti-insulin antibodies compared to pre-diabetic animals (FIG. 18). While no statistically significant difference was observed among the different populations, mice of the COMBO arm displayed a trend towards lower levels. Mice of the 71D6 arm could be clearly divided into 2 subpopulations with low and high auto-antibodies levels, respectively. While these results warrant further investigation, they overall strengthen the hypothesis that neither anti-CD3 antibodies nor 71D6 treatment affect the production of auto-antibodies in this system, but rather act downstream to prevent or delay the onset of diabetes.

Altogether, the data obtained in this set of experiments suggest that 71D6 treatment is very effective in maintaining pancreatic beta cell integrity in the context of type 1 diabetes. Not only systemic 71D6 treatment was significantly more effective than an established immune-suppressing therapy, but it also increased the efficacy of the latter when administered in combination. The mechanism of action underlying the therapeutic activity of 71D6 seems to be related to its ability to promote beta cell survival and/or proliferation rather than interfering with the production of auto-antibodies or the infiltration of immune cells into the pancreatic islets. These data provide experimental evidence that MET-agonistic antibodies can be used in the treatment of type 1 diabetes, alone or in combination with immune therapy.

1-33. (canceled)

34. A method of increasing pancreatic islet cell growth comprising administering to a subject an HGF-MET agonist.

35. The method according to claim 34 wherein the method is used to promote insulin production or treat diabetes in a subject in need thereof.

36. The method according to claim 34, wherein the subject exhibits a fasting glucose level of greater than 5.6 mmol/l.

37. The method according to claim 34, wherein the subject has a population of pancreatic islet cells ranging at least about 50% smaller than the population in a healthy individual to about 80% smaller.

38. The method according to claim 34, wherein the subject has type 1 diabetes, type 2 diabetes, or has previously received a pancreatic tissue transplant.

39. The method according to claim 34, further comprising administering a pancreatic tissue transplant to the subject, or administering one or more immunosuppressive agents to the subject.

40. The method according to claim 39 wherein the one or more immunosuppressive agents are selected from the group consisting of: an anti-CD3 antibody, an anti-IL-21 antibody, a CTLA4 molecule, a PD-L1 molecule, IL-10, and Glutamic Acid Decarboxylase (GAD)-65.

41. The method according to claim 34, wherein the subject is a healthy donor of pancreatic islet cells.

42. The method according to claim 34, wherein administration of the HGF-MET agonist promotes growth of pancreatic islet beta cells.

43. The method of claim 34, wherein the HGF-MET agonist is administered at a dose in the range from 0.1-40 mg/kg per dose.

44. The method of claim 34 wherein the HGF-MET agonist is administered at a dose of 1 mg/kg, 3 mg/kg, 10 mg/kg, or 30 mg/kg.

45. The method according to claim 34, wherein the HGF-MET agonist is administered 1-3 times per week.

46. The method according to claim 34 wherein the method further comprises administering insulin or other anti-diabetes medication to the subject.

47. The method according to claim 34 wherein the HGF-MET agonist is an anti-MET agonist antibody or antigen-binding fragment thereof.

48. The method according to claim 47 wherein the anti-MET antibody or antigen-binding fragment thereof binds to a material selected from the group consisting of a SEMA domain of MET, blades 4-5 of the SEMA [3-propeller, and an epitope comprising a residue selected from the group consisting of Ile367 and Asp372 of MET.

49. The method according to claim 47 wherein the anti-MET antibody or antigen-binding fragment thereof binds to the PSI domain of MET and/or binds an epitope between residues 546 and 562 of MET.

50. The method according to claim 47 wherein the anti-MET antibody or antigen-binding fragment thereof binds to an epitope comprising residue Thr555 of MET.

51. The method according to claim 47 wherein the anti-MET agonist antibody or antigen-binding fragment comprises the combination of HCDR1 consisting of SEQ ID NO: 30, HCDR2 consisting of SEQ ID NO: 32, HCDR3 consisting of SEQ ID NO: 34, LCDR1 consisting of SEQ ID NO: 107, LCDR2 consisting of SEQ ID NO: 109, and LCDR3 consisting of SEQ ID NO: 111.

52. The method according to claim 47 wherein the anti-MET agonist antibody or antigen-binding fragment comprises a VH domain at least 90% identical to SEQ ID NO: 163 and/or comprises a VL domain at least 90% identical to SEQ ID NO: 164.

53. The method according to claim 47, wherein the anti-MET agonist antibody is selected from the group consisting of an agonist antibody or antigen-binding fragment comprising a VH domain consisting of SEQ ID NO: 163 and a VL domain consisting of SEQ ID NO: 164, and an IgG4 antibody.

54. The method according to claim 47 further comprising administering insulin to the subject at least daily.

55. A pharmaceutical composition capable of being used in the method according to claim 34, wherein the pharmaceutical composition comprises an HGF-MET agonist and a pharmaceutically acceptable excipient or carrier.

56. An in vitro method of promoting growth of a cell population or tissue comprising pancreatic islet cells, the method comprising contacting the cell population or tissue with an HGF-MET agonist.

57. An ex vivo method of preserving an islet cell or pancreas transplant which comprises contacting the islet cell or pancreas transplant with an HGF-MET agonist.


SEQUENCE LISTING (210 sequences)

SEQ ID NO: 1 - protein, 30 aa, Lama glama
QLQLVESGGGLVQPGGSLRVSCTASGFTFN

SEQ ID NO: 2 - protein, 5 aa, Lama glama
TYYMT

SEQ ID NO: 3 - protein, 14 aa, Lama glama
WVRQAPGKGLEWVS

SEQ ID NO: 4 - protein, 16 aa, Lama glama
DINSGGGTYYADSVKG

SEQ ID NO: 5 - protein, 32 aa, Lama glama
RFTISRDNAKNTLYLQMNSLKPEDTALYYCVR

SEQ ID NO: 6 - protein, 10 aa, Lama glama
VRIWPVGYDY

SEQ ID NO: 7 - protein, 11 aa, Lama glama
WGQGTQVTVSS

SEQ ID NO: 8 - protein, 30 aa, Lama glama
QVQLVESGGGLVQPGGSLRVSCAASGFTFS

SEQ ID NO: 9 - protein, 5 aa, Lama glama
TYYMS

SEQ ID NO: 10 - protein, 14 aa, Lama glama
WVRQAPGKGLEWVS

SEQ ID NO: 11 - protein, 16 aa, Lama glama
DIRTDGGTYYADSVKG

SEQ ID NO: 12 - protein, 32 aa, Lama glama
RFTMSRDNAKNTLYLQMNSLKPEDTALYYCAR

SEQ ID NO: 13 - protein, 10 aa, Lama glama
TRIFPSGYDY

SEQ ID NO: 14 - protein, 11 aa, Lama glama
WGQGTQVTVSS

SEQ ID NO: 15 - protein, 30 aa, Lama glama
QLQLVESGGGLVQPGGSLRLSCAASGFTFS

SEQ ID NO: 16 - protein, 5 aa, Lama glama
SHAMS

SEQ ID NO: 17 - protein, 14 aa, Lama glama
WVRQAPGKGLEWVS

SEQ ID NO: 18 - protein, 17 aa, Lama glama
AINSGGGSTSYADSVKG

SEQ ID NO: 19 - protein, 32 aa, Lama glama
RFTISRDNAKNTLYLQMNSLKPEDTAVYYCAK

SEQ ID NO: 20 - protein, 17 aa, Lama glama
ELRFDLARYTDYEAWDY

SEQ ID NO: 21 - protein, 11 aa, Lama glama
WGQGTQVTVSS

SEQ ID NO: 22 - protein, 30 aa, Lama glama
ELQLVESGGGLVQPGGSLRLSCAASGFTFS

SEQ ID NO: 23 - protein, 5 aa, Lama glama
GYGMS

SEQ ID NO: 24 - protein, 14 aa, Lama glama
WVRQAPGKGLEWVS

SEQ ID NO: 25 - protein, 17 aa, Lama glama
DINSGGGSTSYADSVKG

SEQ ID NO: 26 - protein, 32 aa, Lama glama; identical to SEQ ID NO: 19

SEQ ID NO: 27 - protein, 17 aa, Lama glama
DMRLYLARYNDYEAWDY

SEQ ID NO: 28 - protein, 11 aa, Lama glama
WGQGTQVTVSS

SEQ ID NO: 29 - protein, 30 aa, Lama glama; identical to SEQ ID NO: 22

SEQ ID NO: 30 - protein, 5 aa, Lama glama
SYGMS

SEQ ID NO: 31 - protein, 14 aa, Lama glama
WVRQAPGKGLEWVS

SEQ ID NO: 32 - protein, 17 aa, Lama glama
AINSYGGSTSYADSVKG

SEQ ID NO: 33 - protein, 32 aa, Lama glama; identical to SEQ ID NO: 19

SEQ ID NO: 34 - protein, 17 aa, Lama glama
EVRADLSRYNDYESYDY

SEQ ID NO: 35 - protein, 11 aa, Lama glama
WGQGTQVTVSS

SEQ ID NO: 36 - protein, 30 aa, Lama glama
EVQLVESGGGLVQPGGSLRLSCAASGFSFK

SEQ ID NO: 37 - protein, 5 aa, Lama glama
DYDIT

SEQ ID NO: 38 - protein, 14 aa, Lama glama
WVRQAPGKGLEWVS

SEQ ID NO: 39 - protein, 17 aa, Lama glama
TITSRSGSTSYVDSVKG

SEQ ID NO: 40 - protein, 32 aa, Lama glama
RFTISGDNAKNTLYLQMNSLKPEDTAVYYCAK

SEQ ID NO: 41 - protein, 17 aa, Lama glama
VYATTWDVGPLGYGMDY

SEQ ID NO: 42 - protein, 11 aa, Lama glama
WGKGTLVTVSS

SEQ ID NO: 43 - protein, 30 aa, Lama glama
EVQLQESGGGLVQPGGSLRLSCAASGFTFS

SEQ ID NO: 44 - protein, 5 aa, Lama glama
IYDMS

SEQ ID NO: 45 - protein, 14 aa, Lama glama
WVRQAPGKGLEWVS

SEQ ID NO: 46 - protein, 17 aa, Lama glama
TINSDGSSTSYVDSVKG

SEQ ID NO: 47 - protein, 32 aa, Lama glama; identical to SEQ ID NO: 19

SEQ ID NO: 48 - protein, 17 aa, Lama glama
VYGSTWDVGPMGYGMDY

SEQ ID NO: 49 - protein, 11 aa, Lama glama
WGKGTLVTVSS

SEQ ID NO: 50 - protein, 30 aa, Lama glama
QVQLVESGGNLVQPGGSLRLSCAASGFTFS

SEQ ID NO: 51 - protein, 5 aa, Lama glama
NYYMS

SEQ ID NO: 52 - protein, 14 aa, Lama glama
WVRQAPGKGLEWVS

SEQ ID NO: 53 - protein, 17 aa, Lama glama
DIYSDGSTTWYSDSVKG

SEQ ID NO: 54 - protein, 32 aa, Lama glama
RFTISRDNAKNTLSLQMNSLKSEDTAVYYCAR

SEQ ID NO: 55 - protein, 10 aa, Lama glama
VKIYPGGYDA

SEQ ID NO: 56 - protein, 11 aa, Lama glama
WGQGTQVTVSS

SEQ ID NO: 57 - protein, 30 aa, Lama glama
QVQLQESGGDLVQPGGSLRVSCVVSGFTFS

SEQ ID NO: 58 - protein, 5 aa, Lama glama
RYYMS

SEQ ID NO: 59 - protein, 14 aa, Lama glama
WVRQAPGKGLEWVS

SEQ ID NO: 60 - protein, 17 aa, Lama glama
SIDSYGYSTYYTDSVKG

SEQ ID NO: 61 - protein, 32 aa, Lama glama
RFTISRDNAKNTLYLQMNSLKPEDTALYYCAR

SEQ ID NO: 62 - protein, 9 aa, Lama glama
AKTTWSYDY

SEQ ID NO: 63 - protein, 11 aa, Lama glama
WGQGTQVTVSS

SEQ ID NO: 64 - protein, 30 aa, Lama glama
EVQLVESGGGLVQPGGSLRLSCAASGFTFR

SEQ ID NO: 65 - protein, 5 aa, Lama glama
NYHMS

SEQ ID NO: 66 - protein, 14 aa, Lama glama
WVRQVPGKGFEWIS

SEQ ID NO: 67 - protein, 17 aa, Lama glama
DINSAGGSTYYADSVKG

SEQ ID NO: 68 - protein, 32 aa, Lama glama
RFTISRDNAKNTLYLEMNSLKPEDTALYYCAR

SEQ ID NO: 69 - protein, 8 aa, Lama glama
VNVWGVNY

SEQ ID NO: 70 - protein, 11 aa, Lama glama
WGKGTLVSVSS

SEQ ID NO: 71 - protein, 30 aa, Lama glama; identical to SEQ ID NO: 22

SEQ ID NO: 72 - protein, 5 aa, Lama glama
NYVMS

SEQ ID NO: 73 - protein, 14 aa, Lama glama
WVRQAPGKGLEWVS

SEQ ID NO: 74 - protein, 16 aa, Lama glama
DTNSGGSTSYADSVKG

SEQ ID NO: 75 - protein, 32 aa, Lama glama; identical to SEQ ID NO: 61

SEQ ID NO: 76 - protein, 8 aa, Lama glama
SFFYGMNY

SEQ ID NO: 77 - protein, 11 aa, Lama glama
WGKGTQVTVSS

SEQ ID NO: 78 - protein, 22 aa, Lama glama
QAVVTQEPSLSVSPGGTVTLTC

SEQ ID NO: 79 - protein, 14 aa, Lama glama
GLSSGSVTTSNYPG

SEQ ID NO: 80 - protein, 15 aa, Lama glama
WFQQTPGQAPRTLIY

SEQ ID NO: 81 - protein, 7 aa, Lama glama
NTNNRHS

SEQ ID NO: 82 - protein, 32 aa, Lama glama
GVPSRFSGSISGNKAALTITGAQPEDEADYYC

SEQ ID NO: 83 - protein, 10 aa, Lama glama
SLYTGSYTTV

SEQ ID NO: 84 - protein, 10 aa, Lama glama
FGGGTHLTVL

SEQ ID NO: 85 - protein, 22 aa, Lama glama
QAVVTQEPSLSVSPGGTVTLTC

SEQ ID NO: 86 - protein, 14 aa, Lama glama
GLSSGSVTTSNYPG

SEQ ID NO: 87 - protein, 15 aa, Lama glama
WFQQTPGQAPRTLIY

SEQ ID NO: 88 - protein, 7 aa, Lama glama
NTNSRHS

SEQ ID NO: 89 - protein, 32 aa, Lama glama
GVPSRFSGSISGNKAALTIMGAQPEDEADYYC

SEQ ID NO: 90 - protein, 9 aa, Lama glama
SLYPGSTTV

SEQ ID NO: 91 - protein, 10 aa, Lama glama
FGGGTHLTVL

SEQ ID NO: 92 - protein, 22 aa, Lama glama
SYELTQPSALSVTLGQTAKITC

SEQ ID NO: 93 - protein, 11 aa, Lama glama
QGGSLGSSYAH

SEQ ID NO: 94 - protein, 15 aa, Lama glama
WYQQKPGQAPVLVIY

SEQ ID NO: 95 - protein, 7 aa, Lama glama
DDDSRPS

SEQ ID NO: 96 - protein, 32 aa, Lama glama
GIPERFSGSSSGGTATLTISGAQAEDEGDYYC

SEQ ID NO: 97 - protein, 11 aa, Lama glama
QSADSSGNAAV

SEQ ID NO: 98 - protein, 10 aa, Lama glama
FGGGTHLTVL

SEQ ID NO: 99 - protein, 22 aa, Lama glama
SSALTQPSALSVTLGQTAKITC

SEQ ID NO: 100 - protein, 11 aa, Lama glama
QGGSLGSSYAH

SEQ ID NO: 101 - protein, 15 aa, Lama glama
WYQQKPGQAPVLVIY

SEQ ID NO: 102 - protein, 7 aa, Lama glama
DDDSRPS

SEQ ID NO: 103 - protein, 32 aa, Lama glama; identical to SEQ ID NO: 96

SEQ ID NO: 104 - protein, 11 aa, Lama glama
QSADSSGNAAV

SEQ ID NO: 105 - protein, 10 aa, Lama glama
FGGGTHLTVL

SEQ ID NO: 106 - protein, 22 aa, Lama glama
QPVLNQPSALSVTLGQTAKITC

SEQ ID NO: 107 - protein, 11 aa, Lama glama
QGGSLGARYAH

SEQ ID NO: 108 - protein, 15 aa, Lama glama
WYQQKPGQAPVLVIY

SEQ ID NO: 109 - protein, 7 aa, Lama glama
DDDSRPS

SEQ ID NO: 110 - protein, 32 aa, Lama glama; identical to SEQ ID NO: 96

SEQ ID NO: 111 - protein, 9 aa, Lama glama
QSADSSGSV

SEQ ID NO: 112 - protein, 10 aa, Lama glama
FGGGTHLTVL

SEQ ID NO: 113 - protein, 22 aa, Lama glama
SYELTQPSALSVTLGQTAKITC

SEQ ID NO: 114 - protein, 11 aa, Lama glama
QGGSLGSSYAH

SEQ ID NO: 115 - protein, 15 aa, Lama glama
WYQQKPGQAPVLVIY

SEQ ID NO: 116 - protein, 7 aa, Lama glama
DDDSRPS

SEQ ID NO: 117 - protein, 32 aa, Lama glama; identical to SEQ ID NO: 96

SEQ ID NO: 118 - protein, 11 aa, Lama glama
QSADSSGNAAV

SEQ ID NO: 119 - protein, 10 aa, Lama glama
FGGGTHLTVL

SEQ ID NO: 120 - protein, 22 aa, Lama glama
SSALTQPSALSVSLGQTARITC

SEQ ID NO: 121 - protein, 11 aa, Lama glama
QGGSLGSSYAH

SEQ ID NO: 122 - protein, 15 aa, Lama glama
WYQQKPGQAPVLVIY

SEQ ID NO: 123 - protein, 7 aa, Lama glama
GDDSRPS

SEQ ID NO: 124 - protein, 32 aa, Lama glama
GIPERFSGSSSGGTATLTISGAQAEDEDDYYC

SEQ ID NO: 125 - protein, 10 aa, Lama glama
QSTDSSGNTV

SEQ ID NO: 126 - protein, 10 aa, Lama glama
FGGGTRLTVL

SEQ ID NO: 127 - protein, 22 aa, Lama glama
QAGLTQPPSVSGSPGKTVTISC

SEQ ID NO: 128 - protein, 14 aa, Lama glama
AGNSSDVGYGNYVS

SEQ ID NO: 129 - protein, 15 aa, Lama glama
WYQQFPGMAPKLLIY

SEQ ID NO: 130 - protein, 7 aa, Lama glama
LVNKRAS

SEQ ID NO: 131 - protein, 32 aa, Lama glama
GITDRFSGSKSGNTASLTISGLQSEDEADYYC

SEQ ID NO: 132 - protein, 10 aa, Lama glama
ASYTGSNNIV

SEQ ID NO: 133 - protein, 10 aa, Lama glama
FGGGTHLTVL

SEQ ID NO: 134 - protein, 23 aa, Lama glama
EIVLTQSPSSVTASVGGKVTINC

SEQ ID NO: 135 - protein, 17 aa, Lama glama
KSSQSVFIASNQKTYLN

SEQ ID NO: 136 - protein, 15 aa, Lama glama
WYQQRPGQSPRLVIS

SEQ ID NO: 137 - protein, 7 aa, Lama glama
YASTRES

SEQ ID NO: 138 - protein, 32 aa, Lama glama
GIPDRFSGSGSTTDFTLTISSVQPEDAAVYYC

SEQ ID NO: 139 - protein, 8 aa, Lama glama
QQAYSHPT

SEQ ID NO: 140 - protein, 10 aa, Lama glama
FGQGTKVELK

SEQ ID NO: 141 - protein, 22 aa, Lama glama
QTVVTQEPSLSVSPGGTVTLTC

SEQ ID NO: 142 - protein, 14 aa, Lama glama
GLSSGSVTTSNYPG

SEQ ID NO: 143 - protein, 15 aa, Lama glama
WFQQTPGQAPRTLIY

SEQ ID NO: 144 - protein, 7 aa, Lama glama
NTNSRHS

SEQ ID NO: 145 - protein, 32 aa, Lama glama; identical to SEQ ID NO: 82

SEQ ID NO: 146 - protein, 10 aa, Lama glama
SLYPGSYTNV

SEQ ID NO: 147 - protein, 10 aa, Lama glama
FGGGTHLTVL

SEQ ID NO: 148 - protein, 22 aa, Lama glama
QSALTQPPSLSASPGSSVRLTC

SEQ ID NO: 149 - protein, 14 aa, Lama glama
TLSSGNNIGSYDIS

SEQ ID NO: 150 - protein, 15 aa, Lama glama
WYQQKAGSPPRYLLN

SEQ ID NO: 151 - protein, 11 aa, Lama glama
YYTDSRKHQDS

SEQ ID NO: 152 - protein, 34 aa, Lama glama
GVPSRFSGSKDASANAGLLLISGLQPEDEADYYC

SEQ ID NO: 153 - protein, 11 aa, Lama glama
SAYKSGSYRWV

SEQ ID NO: 154 - protein, 10 aa, Lama glama
FGGGTHVTVL

SEQ ID NO: 155 - protein, 118 aa, Lama glama
QLQLVESGGGLVQPGGSLRVSCTASGFTFNTYYMTWVRQAPGKGLEWVSDINSGGGTYYADSVKGRFTISRDNAKNTLYLQMNSLKPEDTALYYCVRVRIWPVGYDYWGQGTQVTVSS

SEQ ID NO: 156 - protein, 110 aa, Lama glama
QAVVTQEPSLSVSPGGTVTLTCGLSSGSVTTSNYPGWFQQTPGQAPRTLIYNTNNRHSGVPSRFSGSISGNKAALTITGAQPEDEADYYCSLYTGSYTTVFGGGTHLTVL

SEQ ID NO: 157 - protein, 118 aa, Lama glama
QVQLVESGGGLVQPGGSLRVSCAASGFTFSTYYMSWVRQAPGKGLEWVSDIRTDGGTYYADSVKGRFTMSRDNAKNTLYLQMNSLKPEDTALYYCARTRIFPSGYDYWGQGTQVTVSS

SEQ ID NO: 158 - protein, 109 aa, Lama glama
QAVVTQEPSLSVSPGGTVTLTCGLSSGSVTTSNYPGWFQQTPGQAPRTLIYNTNSRHSGVPSRFSGSISGNKAALTIMGAQPEDEADYYCSLYPGSTTVFGGGTHLTVL

SEQ ID NO: 159 - protein, 126 aa, Lama glama
QLQLVESGGGLVQPGGSLRLSCAASGFTFSSHAMSWVRQAPGKGLEWVSAINSGGGSTSYADSVKGRFTISRDNAKNTLYLQMNSLKPEDTAVYYCAKELRFDLARYTDYEAWDYWGQGTQVTVSS

SEQ ID NO: 160 - protein, 108 aa, Lama glama
SYELTQPSALSVTLGQTAKITCQGGSLGSSYAHWYQQKPGQAPVLVIYDDDSRPSGIPERFSGSSSGGTATLTISGAQAEDEGDYYCQSADSSGNAAVFGGGTHLTVL

SEQ ID NO: 161 - protein, 126 aa, Lama glama
ELQLVESGGGLVQPGGSLRLSCAASGFTFSGYGMSWVRQAPGKGLEWVSDINSGGGSTSYADSVKGRFTISRDNAKNTLYLQMNSLKPEDTAVYYCAKDMRLYLARYNDYEAWDYWGQGTQVTVSS

SEQ ID NO: 162 - protein, 108 aa, Lama glama
SSALTQPSALSVTLGQTAKITCQGGSLGSSYAHWYQQKPGQAPVLVIYDDDSRPSGIPERFSGSSSGGTATLTISGAQAEDEGDYYCQSADSSGNAAVFGGGTHLTVL

SEQ ID NO: 163 - protein, 126 aa, Lama glama
ELQLVESGGGLVQPGGSLRLSCAASGFTFSSYGMSWVRQAPGKGLEWVSAINSYGGSTSYADSVKGRFTISRDNAKNTLYLQMNSLKPEDTAVYYCAKEVRADLSRYNDYESYDYWGQGTQVTVSS

SEQ ID NO: 164 - protein, 106 aa, Lama glama
QPVLNQPSALSVTLGQTAKITCQGGSLGARYAHWYQQKPGQAPVLVIYDDDSRPSGIPERFSGSSSGGTATLTISGAQAEDEGDYYCQSADSSGSVFGGGTHLTVL

SEQ ID NO: 165 - protein, 126 aa, Lama glama
EVQLVESGGGLVQPGGSLRLSCAASGFSFKDYDITWVRQAPGKGLEWVSTITSRSGSTSYVDSVKGRFTISGDNAKNTLYLQMNSLKPEDTAVYYCAKVYATTWDVGPLGYGMDYWGKGTLVTVSS

SEQ ID NO: 166 - protein, 108 aa, Lama glama; identical to SEQ ID NO: 160

SEQ ID NO: 167 - protein, 126 aa, Lama glama
EVQLQESGGGLVQPGGSLRLSCAASGFTFSIYDMSWVRQAPGKGLEWVSTINSDGSSTSYVDSVKGRFTISRDNAKNTLYLQMNSLKPEDTAVYYCAKVYGSTWDVGPMGYGMDYWGKGTLVTVSS

SEQ ID NO: 168 - protein, 107 aa, Lama glama
SSALTQPSALSVSLGQTARITCQGGSLGSSYAHWYQQKPGQAPVLVIYGDDSRPSGIPERFSGSSSGGTATLTISGAQAEDEDDYYCQSTDSSGNTVFGGGTRLTVL

SEQ ID NO: 169 - protein, 119 aa, Lama glama
QVQLVESGGNLVQPGGSLRLSCAASGFTFSNYYMSWVRQAPGKGLEWVSDIYSDGSTTWYSDSVKGRFTISRDNAKNTLSLQMNSLKSEDTAVYYCARVKIYPGGYDAWGQGTQVTVSS

SEQ ID NO: 170 - protein, 110 aa, Lama glama
QAGLTQPPSVSGSPGKTVTISCAGNSSDVGYGNYVSWYQQFPGMAPKLLIYLVNKRASGITDRFSGSKSGNTASLTISGLQSEDEADYYCASYTGSNNIVFGGGTHLTVL

SEQ ID NO: 171 - protein, 118 aa, Lama glama
QVQLQESGGDLVQPGGSLRVSCVVSGFTFSRYYMSWVRQAPGKGLEWVSSIDSYGYSTYYTDSVKGRFTISRDNAKNTLYLQMNSLKPEDTALYYCARAKTTWSYDYWGQGTQVTVSS

SEQ ID NO: 172 - protein, 112 aa, Lama glama
EIVLTQSPSSVTASVGGKVTINCKSSQSVFIASNQKTYLNWYQQRPGQSPRLVISYASTRESGIPDRFSGSGSTTDFTLTISSVQPEDAAVYYCQQAYSHPTFGQGTKVELK

SEQ ID NO: 173 - protein, 117 aa, Lama glama
EVQLVESGGGLVQPGGSLRLSCAASGFTFRNYHMSWVRQVPGKGFEWISDINSAGGSTYYADSVKGRFTISRDNAKNTLYLEMNSLKPEDTALYYCARVNVWGVNYWGKGTLVSVSS

SEQ ID NO: 174 - protein, 110 aa, Lama glama
QTVVTQEPSLSVSPGGTVTLTCGLSSGSVTTSNYPGWFQQTPGQAPRTLIYNTNSRHSGVPSRFSGSISGNKAALTITGAQPEDEADYYCSLYPGSYTNVFGGGTHLTVL

SEQ ID NO: 175 - protein, 116 aa, Lama glama
ELQLVESGGGLVQPGGSLRLSCAASGFTFSNYVMSWVRQAPGKGLEWVSDTNSGGSTSYADSVKGRFTISRDNAKNTLYLQMNSLKPEDTALYYCARSFFYGMNYWGKGTQVTVSS

SEQ ID NO: 176 - protein, 117 aa, Lama glama
QSALTQPPSLSASPGSSVRLTCTLSSGNNIGSYDISWYQQKAGSPPRYLLNYYTDSRKHQDSGVPSRFSGSKDASANAGLLLISGLQPEDEADYYCSAYKSGSYRWVFGGGTHVTVL

SEQ ID NO: 177 - DNA, 354 bp, Lama glama
cagttgcagctggtggagtctgggggaggcttggtgcagcctggggggtctctgagagtttcctgtacagcctctggattcaccttcaatacctactacatgacctgggtccgccaggctccagggaaggggctcgagtgggtctcagatattaatagtggtggtggtacatactatgcagactccgtgaagggccgattcaccatctccagagacaacgccaagaacacgctatatctgcaaatgaacagcctgaaacctgaggacacggccctgtattactgtgtaagagttcgtatttggccagtgggatatgactactggggccaggggacccaggtcaccgtttcctca

SEQ ID NO: 178 - DNA, 330 bp, Lama glama
caggctgtggtgacccaggagccgtccctgtcagtgtctccaggagggacggtcacactcacctgcggcctcagctctgggtctgtcactaccagtaactaccctggttggttccagcagacaccgggccaggctccacgcactcttatctacaacacaaacaaccgccactctggggtccccagtcgcttctccggatccatctctgggaacaaagccgccctcaccatcacgggggcccagcccgaggacgaggccgactattactgttctctatatactggcagttacactactgtgttcggcggagggacccatctgaccgtcctg

SEQ ID NO: 179 - DNA, 354 bp, Lama glama
caggtgcagctcgtggagtctgggggaggcttggtgcagcctggggggtctctgagagtctcctgtgcagcctctggattcaccttcagtacctactacatgagctgggtccgccaggctccagggaaggggctcgagtgggtctcagatattcgtactgatggtggcacatactatgcagactccgtgaagggccgattcaccatgtccagagacaacgccaagaacacgctgtatctacaaatgaacagcctgaaacctgaggacacggccctgtattactgtgcaagaactcgaattttcccctcggggtatgactactggggccaggggacccaggtcaccgtctcctca

SEQ ID NO: 180 - DNA, 327 bp, Lama glama
caggctgtggtgacccaggagccgtccctgtcagtgtctccaggagggacggtcacactcacctgcggcctcagctctgggtctgtcactaccagtaactaccctggttggttccagcagacaccaggccaggctccgcgcactcttatctacaacacaaacagccgccactctggggtccccagtcgcttctccggatccatctctgggaacaaagccgccctcaccatcatgggggcccagcccgaggacgaggccgactattactgttctctgtaccctggtagtaccactgtgttcggcggagggacccatctgaccgtcctg

SEQ ID NO: 181 - DNA, 378 bp, Lama glama
cagttgcagctggtggagtctgggggaggcttggtgcagcctggggggtctctgagactctcctgtgcagcctctggattcaccttcagtagccatgccatgagctgggtccgccaggctccaggaaaggggctcgagtgggtctcagctattaatagtggtggtggtagcacaagctatgcagactccgtgaagggccgattcaccatctccagagacaacgccaagaacacgctgtacctgcaaatgaacagcctgaaacctgaggacacggccgtgtattactgtgcaaaagagctgagattcgacctagcaaggtataccgactatgaggcctgggactactggggccaggggacccaggtcaccgtctcctca

SEQ ID NO: 182 - DNA, 324 bp, Lama glama
tcctatgagctgactcagccctccgcgctgtccgtaaccttgggacagacggccaagatcacctgccaaggtggcagcttaggtagcagttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggtaatgctgctgtgttcggcggagggacccatctgaccgtcctg

SEQ ID NO: 183 - DNA, 378 bp, Lama glama
gagttgcagctggtggagtctgggggaggcttggtgcagcctggggggtctctgagactctcctgtgcagcctctggattcaccttcagtggctatggcatgagctgggtccgccaggctccaggaaaggggctcgagtgggtctcagatattaatagtggtggtggtagcacaagctatgcagactccgtgaagggccgattcaccatctccagagacaacgccaagaacacgctgtatctgcaaatgaacagcctgaaacctgaggacacggccgtgtattactgtgcaaaagatatgagattatacctagcaaggtataacgactatgaggcctgggactactggggccaggggacccaggtcaccgtctcctca

SEQ ID NO: 184 - DNA, 324 bp, Lama glama
tcctctgcactgactcagccctccgcgctgtccgtaaccttgggacagacggccaagatcacctgccaaggtggcagcttaggtagcagttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggtaatgctgctgtgttcggcggagggacccatctgaccgtcctg

SEQ ID NO: 185 - DNA, 378 bp, Lama glama
gagttgcagctggtggagtctgggggaggcttggtgcagcctggggggtctctgagactctcctgtgcagcctctggattcaccttcagtagctatggcatgagctgggtccgccaggctccaggaaaggggctcgagtgggtctcagctattaatagttatggtggtagcacaagctatgcagactccgtgaagggccgattcaccatctccagagacaacgccaagaacacgctgtatctgcaaatgaacagcctgaaacctgaggacacggccgtgtattactgtgcaaaagaagtgcgggccgacctaagccgctataacgactatgagtcgtatgactactggggccaggggacccaggtcaccgtctcctca

SEQ ID NO: 186 - DNA, 318 bp, Lama glama
cagccggtgctgaatcagccctccgcgctgtccgtaaccttgggacagacggccaagatcacctgccaaggtggcagcttaggtgcgcgttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggttctgtgttcggcggagggacccatctgaccgtcctg

SEQ ID NO: 187 - DNA, 378 bp, Lama glama
gaggtgcagctcgtggagtctgggggaggcttggtgcagcctggggggtctctgagactctcctgtgcagcctctggattcagcttcaaggactatgacataacctgggtccgccaggctccgggaaaggggctcgagtgggtctcaactattactagtcgtagtggtagcacaagctatgtagactccgtaaagggccgattcaccatctccggagacaacgccaagaacacgctgtatctgcaaatgaacagcctgaaacctgaggacacggccgtgtattactgtgcaaaagtttacgcgactacctgggacgtcggccctctgggctacggcatggactactggggcaaggggaccctggtcaccgtctcctca

SEQ ID NO: 188 - DNA, 324 bp, Lama glama; identical to SEQ ID NO: 182

SEQ ID NO: 189 - DNA, 378 bp, Lama glama
gaggtgcagctgcaggagtcggggggaggcttggtgcagcctggggggtctctgagactctcctgtgcagcctctggattcaccttcagtatatatgacatgagctgggtccgccaggctccaggaaaggggctcgagtgggtctcaactattaatagtgatggtagtagcacaagctatgtagactccgtgaagggccgattcaccatctccagagacaacgccaagaacacgctgtatctgcaaatgaacagcctgaaacctgaggacacggccgtgtattactgtgcgaaagtttacggtagtacctgggacgtcggccctatgggctacggcatggactactggggcaaagggaccctggtcactgtctcctca

SEQ ID NO: 190 - DNA, 321 bp, Lama glama
tcctctgcactgactcagccctccgcgctgtccgtgtccttgggacagacggccaggatcacctgccaaggtggcagcttaggtagcagttatgctcactggtaccagcagaagccaggccaggcccctgtgctggtcatctatggtgatgacagcaggccctcagggatccctgagcggttctctggctccagctctgggggcacagccaccctgaccatcagcggggcccaggccgaggacgaggatgactattactgtcagtcaacagacagcagtggtaatactgtgttcggcggagggacccgactgaccgtcctg

SEQ ID NO: 191 - DNA, 357 bp, Lama glama
caggtgcagctggtggagtctgggggaaacttggtgcagcctgggggttctctgagactctcctgtgcagcctctggattcaccttcagtaactactacatgagctgggtccgccaggctccagggaaggggctggaatgggtgtccgatatttatagtgacggtagtaccacatggtattcagactccgtcaagggccgattcaccatctccagagacaacgccaagaacacgctgtctctgcaaatgaacagtctgaaatctgaggacacggccgtctattactgtgcgcgcgtgaagatctatccgggggggtatgacgcctggggccaggggacccaggtcaccgtctcctca

SEQ ID NO: 192 - DNA, 330 bp, Lama glama
caggcagggctgactcagcctccctccgtgtctgggtctccaggaaagacggtcaccatctcctgtgcaggaaacagcagtgatgttgggtatggaaactatgtctcctggtaccagcagttcccaggaatggcccccaaactcctgatatatctcgtcaataaacgggcctcagggatcactgatcgcttctctggctccaagtcaggcaacacggcctccctgaccatctctgggctccagtctgaggacgaggctgattattactgtgcctcatatacaggtagcaacaatatcgtgttcggcggagggacccatctaaccgtcctc

SEQ ID NO: 193 - DNA, 354 bp, Lama glama
caggtgcagctgcaggagtcggggggagacttggtgcagcctggggggtctctgagagtctcctgtgtagtctctggattcaccttcagtcgctactacatgagctgggtccgccaggctccagggaaggggctcgagtgggtctcatctattgatagttatggttacagcacatactatacagactccgtgaagggccgattcaccatctccagagacaacgccaagaacacgctgtatctgcaaatgaacagcctgaaacctgaggacacggccctgtattactgtgcaagagcgaaaacgacttggagttatgactactggggccaggggacccaggtcaccgtctcctca

SEQ ID NO: 194 - DNA, 336 bp, Lama glama
gaaattgtgttgacgcagtctcccagctccgtgactgcatctgtaggagggaaggtcactatcaactgtaagtccagccagagcgtcttcatagcttctaatcagaaaacctacttaaactggtaccagcagagacctggacagtctccgaggttggtcatcagctatgcgtccacccgtgaatcggggatccctgatcgattcagcggcagtgggtccacaacagatttcactctcacgatcagcagtgtccagcctgaagatgcggccgtgtattactgtcagcaggcttatagccatccaacgttcggccaggggaccaaggtggaactcaaa

SEQ ID NO: 195 - DNA, 351 bp, Lama glama
gaggtgcagctcgtggagtctgggggaggcttggtgcaacctgggggttctctgagactctcctgtgcagcctctggattcaccttcaggaattaccacatgagttgggtccgccaggttccagggaaggggttcgagtggatctcagatattaatagtgcaggtggtagcacatactatgcagactccgtgaagggccgattcaccatctccagagacaacgccaagaacacgctgtatctggaaatgaacagcctgaaacctgaggacacggccctgtattactgtgcaagagtcaacgtctggggggtgaactactggggcaaagggaccctggtcagcgtctcctca

SEQ ID NO: 196 - DNA, 330 bp, Lama glama
cagactgtggtgactcaggagccgtccctgtcagtgtctccaggagggacggtcacactcacctgcggcctcagctctgggtctgtcactaccagtaactaccctggttggttccagcagacaccaggccaggctccacgcactcttatctacaacacaaacagccgccactctggggtccccagtcgcttctccggatccatctctgggaacaaagccgccctcaccatcacgggggcccagcccgaggacgaggccgactattactgttctctgtaccctggtagttacactaatgtgttcggcggagggacccatctgaccgtcctg

SEQ ID NO: 197 - DNA, 348 bp, Lama glama
gagttgcagctggtggagtctgggggaggcttggtgcagcctggggggtctctgagactctcctgtgcagcctctggattcaccttcagcaactatgtcatgagctgggtccgccaggctccaggaaaggggctcgagtgggtctcagatactaatagtggtggtagcacaagctatgcagactccgtgaagggccgattcaccatctctagagacaacgccaagaacacgctgtatttgcaaatgaacagcctgaaacctgaggacacggcattgtattactgtgcgagatcatttttctacggcatgaactactggggcaaagggacccaggtcaccgtgtcctca

SEQ ID NO: 198 - DNA, 351 bp, Lama glama
cagtctgccctgactcagccgccctccctctctgcatctccgggatcatctgtcagactcacctgcaccctgagcagtggaaacaatattggcagctatgacataagttggtaccagcagaaggcagggagccctccccggtacctcctgaactactacaccgactcacgcaagcaccaggactccggggtcccgagccgcttctctgggtccaaagatgcctcggccaacgcagggcttctgctcatctctgggcttcagcccgaggacgaggctgactattactgttctgcatacaagagtggttcttaccgttgggtgttcggcggagggacgcacgtgaccgtcctg

SEQ ID NO: 199 - protein, 10 aa, Homo sapiens
EPKSCDKTHT

SEQ ID NO: 200 - protein, 5 aa, Homo sapiens
CPPCP

SEQ ID NO: 201 - protein, 8 aa, Homo sapiens
APELLGGP

SEQ ID NO: 202 - protein, 12 aa, Homo sapiens
ELKTPLGDTTHT

SEQ ID NO: 203 - protein, 5 aa, Homo sapiens
CPRCP

SEQ ID NO: 204 - protein, 8 aa, Homo sapiens
APELLGGP

SEQ ID NO: 205 - protein, 7 aa, Homo sapiens
ESKYGPP

SEQ ID NO: 206 - protein, 5 aa, Homo sapiens
CPSCP

SEQ ID NO: 207 - protein, 8 aa, Homo sapiens
APEFLGGP

SEQ ID NO: 208 - protein, 3 aa, Homo sapiens
ERK

SEQ ID NO: 209 - protein, 10 aa, Homo sapiens
CCVECPPPCP

SEQ ID NO: 210 - protein, 7 aa, Homo sapiens
APPVAGP